(19) United States
(12) Patent Application Publication
(10) Pub. No.: US 2010/0183598 A1
(43) Pub. Date: Jul. 22, 2010

(54) METHODS OF TREATING CARDIOVASCULAR DISORDERS

(75) Inventors: Joshua Robert Schultz, Ballston Lake, NY (US); Benedikt Vollrath, San Diego, CA (US); Court Turner, San Diego, CA (US)

Correspondence Address: WILSON, SONSINI, GOODRICH & ROSATI, 650 PAGE MILL ROAD, PALO ALTO, CA, 94304-1050, US

(73) Assignee: CAROLUS THERAPEUTICS, INC., LA JOLLA, CA (US)

(21) Appl. No.: 12/617,643

(22) Filed: Nov. 12, 2009

Related U.S. Application Data
(60) Provisional application No. 61/113,979, filed on Nov. 12, 2008.
(60) Provisional application No. 61/115,450, filed on Nov. 17, 2008.

(51) Int. Cl. A61K 38/16; A61K 39/395; A61P 9/10; A61P 9/02; A61P 9/00; A61P 9/12; A61K 31/351; A61K 31/47; A61K 31/40

(52) U.S. Cl. 424/133.1; 514/2; 514/460; 514/313; 514/423

(57) ABSTRACT

Disclosed herein, in certain embodiments, is a method for treating a cardiovascular disorder. In some embodiments, the method comprises co-administering an inhibitor of inflammation and an agent used to treat a cardiovascular disorder.

### CROSS-REFERENCE

This application claims the benefit of U.S. Provisional Application No. 61/113,979, filed Nov. 12, 2008, and U.S. Provisional Application No. 61/115,450, filed Nov. 17, 2008 both of which are incorporated herein by reference.

### BACKGROUND OF THE INVENTION

Most countries face high and increasing rates of cardiovascular disease. Up until the year 2005, it was the number 1 cause of death and disability in the United States and most European countries.

### SUMMARY OF THE INVENTION

We recognize that there is a need to develop methods and compositions for treating cardiovascular disorders that combine (a) a first agent that inhibits inflammation (also referred herein as an anti-inflammatory agent) and treats a cardiovascular disorder with (b) a second agent that otherwise treats a cardiovascular disorder but results (or has, been shown to result) in undesired inflammation (e.g., myositis).

Disclosed herein, in certain embodiments, are methods and pharmaceutical compositions for modulating a disorder of a cardiovascular system synergistic combination of (a) a therapeutically-effective amount of a modulator of MIF; and (b) a second active agent selected from an agent that treats a cardiovascular disorder (the “cardiovascular disorder agent”).

In some embodiments, the combination is synergistic and results in a more efficacious therapy. In some embodiments, the therapy synergistically treats cardiovascular disorders by (a) targeting multiple pathways that result in (either partially or fully) development of a cardiovascular disorder (e.g., LDL concentrations and the chemotaxis of macrophages) and (b) treating and/or ameliorating undesired inflammation (e.g, myositis) resulting from the cardiovascular disorder agent. In some embodiments, the therapy synergistically treats cardiovascular disorders by targeting multiple pathways that result in (either partially or fully) development of a cardiovascular disorder (e.g., LDL concentrations and the chemotaxis of macrophages).

In some embodiments, the modulator of MIF (i.e., a MIF antagonist), and a statin synergistically treat a CVD by (1) decreasing the chemotaxis of leukocytes, and (2) decreasing (either partially or fully) cholesterol synthesis. In some embodiments, first active agent further treats undesired inflammation resulting from administration of the statin.

In some embodiments, the modulator of MIF (i.e., a MIF) and a fibrate synergistically treat a CVD by (1) decreasing the chemotaxis of leukocytes, and (2) increasing the concentration of HDL. In some embodiments, the modulator of MIF also decreases any undesired inflammation resulting from administration of the fibrate.

In some embodiments, the modulator of MIF (i.e., a MIF antagonist) and a ApoA1 modulator synergistically treat a CVD by (1) decreasing the chemotaxis of leukocytes, and (2) increasing the concentration of HDL. In some embodiments, the modulator of MIF also decreases any undesired inflammation resulting from administration of the ApoA1 modulator.

In some embodiments, the modulator of MIF (i.e., a MIF antagonist) and a ACAT modulator synergistically treat a CVD by (1) decreasing the chemotaxis of leukocytes, and (2) decreasing (a) the production and release of apoB-containing lipoproteins and (b) foam cell formation. In some embodiments, the modulator of MIF also decreases any undesired inflammation resulting from administration of the ACAT inhibitor.

In some embodiments, the modulator of MIF (i.e., a MIF antagonist) and a CETP modulator synergistically treat a CVD by (1) decreasing the chemotaxis of leukocytes, and (2) decreasing the transfer cholesterol from HDL cholesterol to LDL. In some embodiments, the modulator of MIF also decreases any undesired inflammation resulting from administration of the CETP inhibitor.

In some embodiments, the modulator of MIF (i.e., a MIF antagonist) and a GP Iib/IIIa receptor antagonist synergistically treat a CVD by (1) decreasing the chemotaxis of leukocytes, and (2) inhibiting platelet aggregation. In some embodiments, the modulator of MIF also decreases any undesired inflammation resulting from administration of the GP IIb/IIIa receptor antagonist.

In some embodiments, the modulator of MIF (i.e., a MIF antagonist) and a P2Y12 receptor antagonist synergistically treat a CVD by (1) decreasing the chemotaxis of leukocytes, and (2) inhibiting platelet aggregation. In some embodiments, the modulator of MIF also decreases any undesired inflammation resulting from administration of the P2Y12 receptor antagonist.

In some embodiments, the modulator of MIF (i.e., a MIF antagonist) and a Lp-PLA2 antagonist synergistically treat a CVD by (1) decreasing the chemotaxis of leukocytes, and (2) inhibiting the formation of biologically active products from oxidized. LDL. In some embodiments, the modulator of MIF also decreases any undesired inflammation resulting from administration of the Lp-PLA2 antagonist.

Disclosed herein, in certain embodiments, is a method of treating a disorder of a cardiovascular system, comprising co-administering to an individual in need thereof a synergistic combination of (a) a therapeutically-effective amount of a modulator of MIF, and (b) a second active agent selected from an agent that treats a cardiovascular disorder. In some embodiments, the modulator of MIF inhibits (i) MIF binding to CXCR2 and CXCR4 and/or (ii) MW-activation of CXCR2 and CXCR4; (iii) any combination of (i) and (ii); or (iv) the ability of MIF to form a homomultimer. In some embodiments, the modulator of MIF inhibits the ability of MIF to form a homotrimer. In some embodiments, the modulator of MIF binds or competes with a pseudo-ELR motif of MIF. In some embodiments, the modulator of MIF inhibits binding of a pseudo-ELR motif of MIF to CXCR2 and/or CXCR4. In some embodiments, the modulator of MIF binds or competes with a N-Loop motif of MIF. In some embodiments, the modulator of MIF inhibits binding of a N-Loop motif of MIF to CXCR2 and/or CXCR4. In some embodiments, the modulator of MIF binds to the pseudo-ELR and N-Loop motif of MIF. In some embodiments, the modulator of MIF is a CXCR2 antagonist; an anti-CXCR2 antibody; a CXCR4 antagonist; an anti-CXCR4 antibody; a MIF antagonist; an anti-MIF antibody; or combinations thereof. In some embodiments, the modulator of MIF is a CXCR2 antagonist selected from CXCL8(3-74)K11R/G31P, Sch527123, N-(3-(aminosulfonyl)-4-chloro-2-hydroxyphenyl)-N′-(2,3-dichlorophenyl)urea, IL-8(1-72), (R)IL-8, (R)IL-8,NMeLeu, (AAR)IL-8, GROα(1-73), (R)GROα, (ELR)PF4, (R)PF4, SB-265610, Antileukinate, SB-517785-M, SB 265610, SB225002, SB455821, DF2162 and Reparixin. In some embodiments, the modulator of MIF is an anti-CXCR2 antibody selected from 48311.211 or a derivative thereof. In some embodiments, the modulator of MIF is a CXCR4 antagonist selected from ALX40-4C, AMD-070, AMD3100, AMD3465, KRH-1636, KRH-2731, KRH-3955, KRH-3140, T134, T22, T140, TC14012, TN14003, RCP168, POL3026, and CTCE-0214. In some embodiments, the modulator of MIF is an anti-CXCR4 antibody selected from 701, 708, 716, 717, 718, 12G5 and 4G10. In some embodiments, the modulator of MIF is an anti-MIF antibody selected from IID.9, IIID.9, XIF7, I31, IV2.2, XI17, XIV14.3, XII15.6 and XIV15.4. In some embodiments, the modulator of MIF is an MIF antagonist selected from COR100140. In some embodiments, administration of the second active agent partially or fully results in undesired inflammation. In some embodiments, the modulator of MIF treats and/or ameliorates the inflammation induced by administration of the second active agent. In some embodiments, co-administering the modulator of MIF with the second active agent rescues the individual from inflammation induced by administration of the second active agent. In some embodiments, the second active agent is niacin; a fibrate; a statin; an apolipoprotein A-1 modulator; an ACAT modulator; a CETP modulator; a glycoprotein 1113/IIIa modulator; a P2Y12 modulator; an Lp-PLA2 modulator; an anti-hypertensive; or combinations thereof. In some embodiments, the second active agent is a statin selected from atorvastatin; cerivastatin; fluvastatin; lovastatin; mevastatin; pitavastatin; pravastatin; rosuvastatin; simvastatin; simvastatin and ezetimibe; lovastatin and niacin, extended-release; atorvastatin and amlodipine besylate; simvastatin and niacin, extended-release; or combinations thereof. In some embodiments, the second active agent is bezafibrate; ciprofibrate; clofibrate; gemfibrozil; fenofibrate; or combinations thereof. In some embodiments, the second active agent is DF4 (Novartis); DF5 (Bruin Pharmaceuticals); RVX-208 (Resverlogix); or combinations thereof. In some embodiments, the second active agent is avasimibe; pactitnibe sulfate (CS-505); CI-1011 (2,6-diisopropylphenyl [(2,4,6-triisopropylphenypacetyl]sulfamate); CI-976 (2,2-dimethyl-N-(2,4,6-trimethoxyphenyl)dodecanamide); VULM1457 (142; 6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N[[4-(2,2-dimethylpropyl)phenyl]methyl]-N-(hepthyl)urea); F-1394 ((1s,2s)-2-[3-(2,2-dimethylpropyl)-3-nonylureido]aminocyclohexane-1-yl 3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; or combinations thereof. In some embodiments, the second active agent is torcetrapib; anacetrapid; JTT-705 (Japan Tobacco/Roche); or combinations thereof. In some embodiments, the second active agent is abciximab; eptifibatide; tirofiban; roxifiban; variabilin; XV 459 (N(3)-(2-(3-(4-formamidinophenyl)isoxazolin-5-yl)acetyl)-N(2)-(1-butyloxycarbonyl)-2,3-diaminopropionate); SR 121566A (3-[N-{4-[4-(aminoiminomethyl)phenyl]-1,3-thiazol-2-yl}-N-(1-carboxymethylpiperid-4-yl)amino]propionic acid, trihydrochloride); FK419 ((S)-2-acetylamino-3-[(R)-[1-[3-(piperidin-4-yl)propionyl]piperidin-3-ylcarbonyl]amino]propionic acid trihydrate); or combinations thereof. In some embodiments, the second active agent is clopidogrel; prasugrel; cangrelor; AZD6140 (AstraZeneca); MRS 2395 (2,2-Dimethyl-propionic acid 3-(2-chloro-6-methylaminopurin-9-yl)-2-(2,2-dimethyl-propionyloxymethyl)-propyl ester); BX 667 (Berlex Biosciences); BX 048 (Berlex Biosciences) or combinations thereof. In some embodiments, the second active agent is darapladib (SB 480848); SB-435-495 (GlaxoSmithKline); SB-222657 (GlaxoSmithKline); SB-253514 (GlaxoSmithKline); or combinations thereof. In some embodiments, the second active agent is a diuretic; a vasodilator; a beta-blocker; a calcium-channel blocker; or combinations thereof. In some embodiments, the second active agent is administered before, after, or simultaneously with the modulator of inflammation. In some embodiments, the disorder is hyperlipidemia; hypercholesterolemia; hyperglyceridemia; combined hyperlipidemia; hypolipoproteinemia; hypocholesterolemia; abetlipoproteinemia; Tangier disease; acute coronary syndrome; unstable angina; non-ST segment elevation myocardial infarction; ST segment elevation myocardial infarction; stable angina; Prinzmetal's angina; arteriosclerosis; atherosclerosis; arteriolosclerosis; stenosis; restenosis; venous thrombosis; arterial thrombosis; stroke; transient ischemic attack; peripheral vascular disease; coronary artery disease; hypertension; or combinations thereof.

Disclosed herein, in certain embodiments, is a method of treating a lipid disorder, comprising (a) removing a lipid from the blood of an individual in need thereof; and (b) administering a therapeutically-effective amount of a modulator of MIF. In some embodiments, administering a therapeutically-effective amount of a modulator of inflammation acts in synergy with the removal of a lipid from the blood of an individual in need thereof. In some embodiments, the modulator of MIF inhibits (i) MIF binding to CXCR2 and CXCR4 and/or (ii) MIF-activation of CXCR2 and CXCR4; (iii) any combination of (i) and (ii); or (iv) the ability of MIF to form a homomultimer. In some embodiments, the modulator of MIF inhibits the ability of MIF to form a homotrimer. In some embodiments, the modulator of MIF binds or competes with a pseudo-ELR motif of MIF. In some embodiments, the modulator of MIF inhibits binding of a pseudo-ELR motif of MIF to CXCR2 and/or CXCR4. In some embodiments, the modulator of MIF binds or competes with a N-Loop motif of MIF. In some embodiments, the modulator of MIF inhibits binding of a N-Loop motif of MIF to CXCR2 and/or CXCR4. In some embodiments, the modulator of MIF binds to the pseudo-ELR and N-Loop motif of MIF. In some embodiments, the modulator of MIF is a CXCR2 antagonist; an anti-CXCR2 antibody; a CXCR4 antagonist; an anti CXCR4 antibody; a MIF antagonist an anti-MIF antibody; or combination s thereof. In some embodiments, the modulator of MIF is a CXCR2 antagonist selected from CXCL8(3-74)K11R/G31P, Sch527123, N-(3-(aminosulfonyl)-4-chloro-2-hydroxyphenyl)-N-(2,3-dichlorophenyl)urea, IL-8(1-72), (R)IL-8, (R)IL-8, NMeLeu, (AAR)IL-8, GROα(1-73), (R)GROα, (ELR)PF4, (R)PF4, SB-265610, Antileukinate, SB-517785-M, SB 265610, SB225002, SB455821, DF2162 and Reparixin. In some embodiments, the modulator of MIF is an anti-CXCR2 antibody selected from 48311.211 or a derivative thereof. In some embodiments, the modulator of MIF is a CXCR4 antagonist selected from ALX40-4C, AMD-070, AMD3100, AMD3465, KRH-1636, KRH-2731, KRH-3955, KRH-3140, T134, T22, T140, TC14012, TN14003, RCP168, POL3026, and CTCE-0214. In some embodiments, the modulator of MIF is an anti-CXCR4 antibody selected from 701, 708, 716, 717, 718, 12G5 and 4G10. In some embodiments, the modulator of MIF is an anti-MIF antibody selected from IID.9, IIID.9, XIF7, I31, IV2.2, XI17, XIV14.3, XII15.6 and XIV15.4. In some embodiments, the modulator of MIF is an MIF antagonist selected from COR100140. In some embodiments, the disorder is hyperlipidemia; hypercholesterolemia; hyperglyceridemia; combined hyperlipidemia; hypolipoproteinemia; hypocholesterolemia; abetlipoproteinemia; Tangier disease; or combinations thereof.

Disclosed herein, in certain embodiments, is a method of treating a lipid disorder, comprising (a) modulating the concentration of a lipid in the blood of an individual in need thereof; and (b) administering a therapeutically-effective amount of a modulator of MIF. In some embodiments, administering a therapeutically-effective amount of a modulator of inflammation acts in synergy with the modulating of the concentration of a lipid. In some embodiments, the method comprises transfecting DNA encoding an Apo A1 gene, an LCAT gene, an LDL gene, or a combination thereof. In some embodiments, the DNA is transfected into a liver cell. In some embodiments, the method comprises silencing the expression of Apolipoprotein B (Apo B), Heat Shock Protein 110 (Hsp 110), Proprotein Convertase Subtilisin Kexin 9 (Pcsk9), or a combination thereof. In some embodiments, silencing the expression of Apolipoprotein B (Apo B), Heat Shock Protein 110 (Hsp 110), Proprotein Convertase Subtilisin Kexin 9 (Pcsk9) comprises the use of an siRNA molecule. In some embodiments, the method comprises modulating the activity of microRNA-122. In some embodiments, modulating the activity of microRNA-122 comprises use of an antisense molecule. In some embodiments, the modulator of MIF inhibits (i) MIF binding to CXCR2 and CXCR4 and/or (ii) MIF-activation of CXCR2 and CXCR4; or (iii) any combination of (i) and (ii). In some embodiments, the modulator of MIF binds or competes with a pseudo-ELR motif of MIF. In some embodiments, the modulator of MIF inhibits binding of a pseudo-ELR motif of MIF to CXCR2 and/or CXCR4. In some embodiments, the modulator of MIF binds or competes with a N-Loop motif of MIF. In some embodiments, the modulator of MIF inhibits binding of a N-Loop motif of MIF to CXCR2 and/or CXCR4. In some embodiments, the modulator of MIF binds to the pseudo-ELR and N-Loop motif of MIF. In some embodiments, the modulator of MIF is a CXCR2 antagonist; an anti-CXCR2 antibody; a CXCR4 antagonist; an anti-CXCR4 antibody; a MIF antagonist; an anti-MIF antibody; or combination s thereof. In some embodiments, the modulator of MIF is a CXCR2 antagonist selected from CXCL8(3-74)K11R/G31P, Sch527123, N-(3-(aminosulfonyl)-4-chloro-2-hydroxyphenyl)-N-(2,3-dichlorophenyl)urea, IL-8(1-72), (R)IL-8, (R)IL-8,NMeLeu, (AAR)IL-8, GROcc(1-73), (R)GROcc, (ELR)PF4, (R)PF4, SB-265610, Antileukinate, SB-517785-M, SB 265610, SB225002, SB455821, DF2162 and Reparixin. In some embodiments, the modulator of MIF is an anti-CXCR2 antibody selected from 48311.211 or a derivative thereof. In some embodiments, the modulator of MIF is a CXCR4 antagonist selected from ALX40-4C, AMD-070, AMD3100, AMD3465, KRH-1636, KRH-2731, KRH-3955, KRH-3140, T134, T22, T140, TC14012, TN14003, RCP168, POL3026, and CTCE-0214. In some embodiments, the modulator of MIF is an anti-CXCR4 antibody selected from 701, 708, 716, 717, 718, 12G5 and 4010. In some embodiments, first active agent is an anti-MIF antibody selected from IID.9, IIID.9, XIF7, I31, IV2.2, XI17, XIV14.3, XII15.6 and XIV15.4. In some embodiments, the modulator of MIF is an MIF antagonist selected from COR100140. In some embodiments, the disorder is hyperlipidemia; hypercholesterolemia; hyperglyceridemia; combined hyperlipidemia; hypolipoproteinemia; hypocholesterolemia; abetlipoproteinemia; Tangier disease; or combinations thereof.

Disclosed herein, in certain embodiments, is a pharmaceutical composition for modulating a disorder of a cardiovascular system, comprising a synergistic combination of (a) a therapeutically-effective amount of an agent that treats a cardiovascular disorder; and (b) a therapeutically-effective amount of a modulator of MIF. In some embodiments, the modulator of a cardiovascular disorder partially or fully causes undesired inflammation. In some embodiments, the modulator of inflammation treats and/or ameliorates inflammation partially or fully cause by from the modulator of a cardiovascular disorder. In some embodiments, the combination of a modulator of inflammation with modulator of a cardiovascular disorder rescues the individual from inflammation partially or fully caused by the modulator of a cardiovascular disorder. In some embodiments, the modulator of MIF inhibits (i) MIF binding to CXCR2 and CXCR4 and/or (ii) MIF-activation of CXCR2 and CXCR4; (iii) any combination of (i) and (ii); or (iv) the ability of MIF to form a homomultimer. In some embodiments, the modulator of MIF inhibits the ability of MIF to form a homotrimer. In some embodiments, the modulator of MIF binds or competes with a pseudo-ELR motif of MIF. In some embodiments, the modulator of MIF inhibits binding of a pseudo-ELR motif of MIF to CXCR2 and/or CXCR4. In some embodiments, the modulator of MIF binds or competes with a N-Loop motif of MIF. In some embodiments, the modulator of MIF inhibits binding of a N-Loop motif of MIF to CXCR2 and/or CXCR4. In some embodiments, the modulator of MIF binds to the pseudo-ELR and N-Loop motif of MIF. In some embodiments, the modulator of MIF is a CXCR2 antagonist; an anti-CXCR2 antibody; a CXCR4 antagonist; an anti-CXCR4 antibody; a MIF antagonist; an anti-MIF antibody; or combination s thereof. In some embodiments, the modulator of MIF is a CXCR2 antagonist selected from CXCL8(3-74)K11R/G31P, Sch527123, N-(3-(aminosulfonyl)-4-chloro-2-hydroxyphenyl)-AP-(2,3-dichlorophenyl)urea, IL-8(1-72), (R)IL-8, (R)IL-8,NMeLeu, (AAR)IL-8, GROα(1-73), (R)GROα, (ELR)PF4, (R)PF4, SB-265610, Antileukinate, SB-517785-M, SB 265610, SB225002, SB455821, DF2162 and Reparixin. In some embodiments, the modulator of MIF is an anti-CXCR2 antibody selected from 48311.211 or a derivative thereof. In some embodiments, the modulator of MIF is a CXCR4 antagonist selected from ALX40-4C, AMD-070, AMD3100, AMD3465; KRH-1636, KRH-2731, KRH-3955, KRH-3140, T134, T22, T140, TC14012, TN14003, RCP168, POL3026, and CTCE-0214. In some embodiments, the modulator of MIF is an anti-CXCR4 antibody selected from 701, 708, 716, 717, 718, 12G5 and 4G10. In some embodiments, first active agent is an anti-MIF antibody selected from IID.9, IIID.9, XIF7, I31, IV2.2, XI17, XIV14.3, XII15.6 and XIV15.4. In some embodiments, the modulator of MIF is an MIF antagonist selected from COR100140. In some embodiments, administration of the second active agent partially or fully results in inflammation. In some embodiments, the modulator of MIF treats and/or ameliorates the inflammation induced by administration of the second active agent. In some embodiments, co-administering the modulator of MIF with the second active agent rescues the individual from inflammation induced by administration of the second active agent. In some embodiments, the second active agent is niacin; a fibrate; a statin; an apolipoprotein A-1 modulator; an ACAT modulator; a CETP modulator; a glycoprotein IIb/IIIa modulator; a P2Y12 modulator; an Lp-PLA2 modulator; an anti-hypertensive; or combinations thereof. In some embodiments, second active agent is a statin selected from atorvastatin; cerivastatin; fluvastatin; lovastatin; mevastatin; pitavastatin; pravastatin; rosuvastatin; simvastatin; simvastatin and ezetimibe; lovastatin and niacin, extended-release; atorvastatin and amlodipine besylate; simvastatin and niacin, extended-release; or combinations thereof. In some embodiments, the second active agent is bezafibrate; ciprofibrate; clofibrate; gemfibrozil; fenofibrate; or combinations thereof. In some embodiments, the second active agent is DF4 (Ac-D-W-F-K-A-F-Y-D-K-V-A-E-K-F-K-E-A-F-NH2); DFS; RVX-208 (Resverlogix); or combinations thereof. In some embodiments, the second active agent is avasimibe; pactimibe sulfate (CS-505); CI-1011 (2,6-diisopropylphenyl[(2,4,6-triisopropylphenypacetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N[[4-(2,2-dimethylpropyl)phenyl]methyl]-N-(hepthyl)urea); F-1394 ((1s,2s)-2-[3-(2,2-dimethylpropyl)-3-nonylureido]aminocyclohexane-1-yl 3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; or combinations thereof. In some embodiments, the CETP modulator is torcetrapib; anacetrapid; JTT-705 (Japan Tobacco/Roche); or combinations thereof. In some embodiments, the second active agent is abciximab; eptifibatide; tirofiban; roxifiban; variabilin; XV 459 (N(3)-(2-(3-(4-formamidinophenyl)isokazolin-5-yl)acetyl)-N(2)-(1-butyloxycarbonyl)-2,3-diaminopropionate); SR 121566A (3-[N-{4-[4-(aminoiminomethyl)phenyl]-1,3-thiazol-2-yl}-N-(1-carboxymethylpiperid-4-yl)amino]propionic acid, trihydrochloride); FK419 ((S)-2-acetylamino-3-[(R)-[1-[3-(piperidin-4-yl)propionyl]piperidin-3-ylcarbonyl]amino]propionic acid trihydrate); or combinations thereof. In some embodiments, the second active agent is clopidogrel; prasugrel; cangrelor; AZD6140 (AstraZeneca); MRS 2395 (2,2-Dimethyl-propionic acid 3-(2-chloro-6-methylaminopurin-9-yl)-2-(2,2-dimethyl-propionyloxymethyl)-propyl ester); BX 667 (Berlex Biosciences); BX 048 (Berlex Biosciences) or combinations thereof. In some embodiments, the second active agent is darapladib (SB 480848); SB-435-495 (GlaxoSmithKline); SB-222657 (GlaxoSmithKline); SB-253514 (GlaxoSmithKline); or combinations thereof. In some embodiments, the second active agent is a diuretic; a vasodilator; a beta-blocker; a calcium-channel blocker; or combinations thereof. In some embodiments, the composition comprises a first population of particles and a second population of particles. In some embodiments, the first population of particles is formulated for immediate release. In some embodiments, the second population of particles is formulated for controlled release.

### DETAILED DESCRIPTION OF THE INVENTION

Disclosed herein, in certain embodiments, are methods and pharmaceutical compositions for modulating a disorder of a cardiovascular system synergistic combination of (a) a therapeutically-effective amount of a modulator of MIF; and (b) a second active agent selected from an agent that treats a cardiovascular disorder. (the “cardiovascular disorder agent”).

In some embodiments, the combination is synergistic and results in a more efficacious therapy. In some embodiments, the therapy synergistically treats cardiovascular disorders by (a) targeting multiple pathways that result in (either partially or fully) development of a cardiovascular disorder (e.g., LDL concentrations and the chemotaxis of macrophages) and (b) treating and/or ameliorating undesired inflammation (e.g, myositis) resulting from the cardiovascular disorder agent. In some embodiments, the therapy synergistically treats cardiovascular disorders by targeting multiple pathways that result in (either partially or fully) development of a cardiovascular disorder (e.g., LDL concentrations and the chemotaxis of macrophages).

In some embodiments, the combination rescues a mammal from inflammation partially or fully caused by the cardiovascular disorder agent. In some embodiments, the combination allows (partially or fully) a medical professional to increase the prescribed dosage of the cardiovascular disorder agent. In some embodiments, the combination enables (partially or fully) a medical professional to prescribe the cardiovascular disorder agent (i.e., co-administration rescues the cardiovascular disorder agent).

In some embodiments, the modulator of MIF (i.e., a MIF antagonist), and a statin synergistically treat a CVD by (1) decreasing the chemotaxis of leukocytes, and (2) decreasing (either partially or fully) cholesterol synthesis. In some embodiments, first active agent further treats undesired inflammation resulting from administration of the statin.

In some embodiments, the modulator of MIF (i.e., a MIF antagonist) and a fibrate synergistically treat a CVD by (1) decreasing the chemotaxis of leukocytes, and (2) increasing the concentration of HDL. In some embodiments, the modulator of MIF also decreases any undesired inflammation resulting from administration of the fibrate.

In some embodiments, the modulator of MIF (i.e., a MIF antagonist) and a ApoA1 modulator synergistically treat a CVD by (1) decreasing the chemotaxis of leukocytes, and (2) increasing the concentration of HDL. In some embodiments, the modulator of MIF also decreases any undesired inflammation resulting from administration of the ApoA1 modulator.

In some embodiments, the modulator of MIF (i.e., a MIF antagonist) and a ACAT modulator synergistically treat a CVD by (1) decreasing the chemotaxis of leukocytes, and (2) decreasing (a) the production and release of apoB-containing lipoproteins and (b) foam cell formation. In some embodiments, the modulator of MIF also decreases any undesired inflammation resulting from administration of the ACAT inhibitor.

In some embodiments, the modulator of MIF (i.e., a MIF antagonist) and a CETP modulator synergistically treat a CVD by (1) decreasing the chemotaxis of leukocytes, and (2) decreasing the transfer cholesterol from HDL cholesterol to LDL. In some embodiments, the modulator of MIF also decreases any undesired inflammation resulting from administration of the CETP inhibitor.

In some embodiments, the modulator of MIF (i.e., a MIF antagonist) and a GP IIb/IIIa receptor antagonist synergistically treat a CVD by (1) decreasing the chemotaxis of leukocytes, and (2) inhibiting platelet aggregation. In some embodiments, the modulator of MIF also decreases any undesired inflammation resulting from administration of the GP IIb/IIIa receptor antagonist.

In some embodiments, the modulator of MIF (i.e., a MIF antagonist) and a P2Y12 receptor antagonist synergistically treat a CVD by (1) decreasing the chemotaxis of leukocytes, and (2) inhibiting platelet aggregation. In some embodiments, the modulator of MIF also decreases any undesired inflammation resulting from administration of the P2Y12 receptor antagonist.

In some embodiments, the modulator of MIF (i.e., a MIF antagonist) and a Lp-PLA2 antagonist synergistically treat a CVD by (1) decreasing the chemotaxis of leukocytes, and (2) inhibiting the formation of biologically active, active from oxidized LDL. In some embodiments, the modulator of MIF also decreases any undesired inflammation resulting from administration of the Lp-PLA2 antagonist.

### CERTAIN DEFINITIONS

The terms “individual,” “individual,” or “subject” are used interchangeably. As used herein, they mean any mammal (i.e. species of any orders, families, and genus within the taxonomic classification animalia: chordata: vertebrata: mammalia). In some embodiments, the mammal is a human. In some embodiments, the mammal is a non-human. In some embodiments, the mammal is a member of the taxonomic orders: primates (e.g. lemurs, lorids, galagos, tarsiers, monkeys, apes, and humans); rodentia (e.g. mice, rats, squirrels, chipmunks, and gophers); lagomorpha (e.g. hares, rabbits, and pika); erinaceomorpha (e.g. hedgehogs and gymnures); soricomorpha (e.g. shrews, moles, and solenodons); chiroptera (e.g.; bats); cetacea (e.g. whales, dolphins, and porpoises); camivora (e.g. cats, lions, and other feliformia; dogs, bears, weasels, and seals); perissodactyla (e.g. horse, zebra, tapir, and rhinoceros); artiodactyla (e.g. pigs, camels, cattle, and deer); proboscidea (e.g, elephants); sirenia (e.g. manatees, dugong, and sea cows); cingulata (e.g. armadillos); pilosa (e.g. anteaters and sloths); didelphimorphia (e.g. american opossumS); paudituberculata (e.g. shrew opossums); microbiotheria (e.g. Monito del Monte); notoryctemotphia (e.g. marsupial moles); dasyuromorphia (e.g. marsupial carnivores); peramelemorphia (e.g. bandicoots and bilbies); or diprotodontia (e.g. wombats, koalas, possums, gliders, kangaroos, wallaroos, and wallabies). In some embodiments, the animal is a reptile (i.e. species of any orders, families, and genus within the taxonomic classification animalia: chordata: vertebrata: reptilia). In sortie embodiments, the animal is a bird (i.e. animalia: chordata: vertebrata: aves). None of the terms require or are limited to situation characterized by the supervision (e.g. constant or intermittent) of a health care worker (e.g. a doctor, a registered nurse, a nurse practitioner, a physician's assistant, an orderly, or a hospice worker).

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or town individual at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to an individual simultaneously in the form of a single entity or dosage. The term “non-fixed combination” means that at least one of the agents described herein, and at least one co-agent, are administered to an individual as separate entities either simultaneously, concurrently or sequentially with variable intervening time limits, wherein such administration provides effective levels of the two or more agents in the body of the individual. In some instances, the co-agent is administered once or for a period of time, after which the agent is administered once or over a period of time. In other instances, the co-agent is administered for a period of time; after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single individual, and are intended to include treatment regimens in which the agents are administered by the same or different route of administration or at the same or different times. In some embodiments the agents described herein will be co-administered with other agents. These terms encompass administration of two or more agents to an animal so that both agents and/of their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer stem cell. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein, include e.g., as discussed in Goodman and Gilman, The Pharmacological Basis of Therapeuties, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

“Pharmaceutically acceptable prodrug” as used herein, refers to any pharmaceutically acceptable salt, ester, salt of an ester or other derivative of an agent, which, upon administration to a recipient, is capable of providing, either directly or indirectly, a agent of this invention or a pharmaceutically active metabolite or residue thereof. Particularly favored prodrugs are those that increase the bioavailability of the agents of this invention when such agents are administered to an individual (e.g., by allowing an orally administered agent to be more readily absorbed into blood) or which enhance delivery of the parent agent to a biological compartment (e.g., the brain or lymphatic system). In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium or potassium), ammonium salts and the like.

The term “recruiting of monocytes” as described herein includes the migration of monocytes into or out of the endothelium, their attachment and propagation, for example, into endothelial fissures. The attachment of monocytes is also known as monocyte adhesion, or as monocyte arrest when the attachment occurs in shear flow as under physiological conditions, for example, in blood capillaries, microvascular or arterial streamlines.

By the term “polypeptide” is meant synthetic or nonsynthetic peptide compounds, as well as purified, modified fragments of natural proteins, native forms or recombinant peptides or proteins. The term “polypeptide” likewise includes pharmacologically acceptable salts, pharmacologically acceptable derivatives and/or conjugates of the corresponding polypeptide.

Pharmacologically acceptable derivatives include, for example, esters, amides, N-acyl and/or O-acyl derivatives, carboxylated, acetylated, phosphorylated and/or glycosylated polypeptides. Conjugates include, for example, sugar or polyethylene glycol conjugates, biotinylated, radioactively or fluorescently labeled polypeptides.

The term “peptide mimetic”, “mimetic peptide” and “analog” are used herein interchangeably for the purposes of the specifications and claims, to mean a peptide that mimics part or all of the bioactivity of an endogenous protein ligand. In one embodiment, peptide mimetics are modeled after a specific peptide and display an altered peptide backbone, altered amino acids and/or an altered primary amino acid sequence when compared to the peptide of which is was designed to mimic.

As used herein, the terms “antibody” and “antibodies” refer to monoclonal antibodies, polyclonal antibodies, bi-specific antibodies, multispecific antibodies, grafted antibodies, human antibodies, humanized antibodies, synthetic antibodies, chimeric antibodies, camelized antibodies, single-chain Fvs (scFv), single chain antibodies, Fab fragments, F(ab′) fragments, disulfide-linked Fvs (sdFv), intrabodies, and anti-idiotypic (anti-Id) antibodies and antigen-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG₁, IgG₂, IgG₃, IgG₄, IgA₁ and IgA₂) or subclass. The terms “antibody” and immunoglobulin are used interchangeably in the broadest sense. In some embodiments an antibody is part of a larger fusion molecule, formed by covalent or non-covalent association of the antibody with one or more other proteins or peptides.

As used herein, the term “derivative” in the context of a polypeptide or protein, e.g. an antibody, refers to a polypeptide or protein that comprises an amino acid sequence which has been altered by the introduction of amino acid residue substitutions, deletions or additions. The term “derivative” as used herein also refers to a polypeptide or protein which has been modified, i.e., by the covalent attachment of any type of molecule to the antibody. For example, in some embodiments a polypeptide or protein is modified, e.g., by glycosylation, acetylation, pegylation, phosphorylation, amidation, derivatization by protecting/blocking groups, proteolytic cleavage, linkage to a cellular ligand or other protein, etc. In some embodiments, derivatives, polypeptides or proteins are produced by chemical modifications using techniques, including, but not limited to specific chemical cleavage, acetylation, formylation, metabolic synthesis of tunicamycin, etc. In some embodiments a derivative a polypeptide or protein possesses a similar or identical function as the polypeptide or protein from which it was derived.

The terms “full length antibody”, “intact antibody” and “whole antibody” are used herein interchangeably, to refer to, an antibody in its substantially intact: form, and not antibody fragments as defined below. These terms particularly refer to an antibody with heavy chains contains Fc regions. In some embodiments an antibody variant of the invention is a full length, antibody. In some embodiments the full length antibody is human, humanized, chimeric, and/or affinity matured.

An “affinity matured” antibody is one having one or more alteration in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures, such as for example, Marks et al., (1992) Biotechnology 10:779-783 that describes affinity maturation by variable heavy chain (VH) and variable light chain (VL) domain shuffling. Random mutagenesis of CDR and/or framework residues is described in: Barbas, et al. (1994) Proc. Nat. Acad. Sci, USA 91:3809-3813; Shier et al., (1995) Gene 169:147-155; Yelton et al., 1995, J. Immunol. 155:1994-2004; Jackson et al., 1995, J. Immunol. 154(7):3310-9; and Hawkins et al, (19920, J. Mol. Biol. 226:889-896, for example.

The terms “binding fragment”, “antibody fragment” or “antigen binding fragment” are used herein, for purposes of the specification and claims, to mean a portion or fragment of an intact antibody molecule, preferably wherein the fragment retains antigen-binding function. Examples of antibody fragments include Fab, Fab′, F(ab′)₂, Fd, Fd′ and Fv fragments, diabodies, linear antibodies (Zapata et al. (1995) Protein Eng. 10: 1057), single-chain antibody molecules, single-chain binding polypeptides, scFv, bivalent scFv, tetravalent scFv, and bispecific or multispecific antibodies formed from antibody fragments.

“Fab” fragments are typically produced by papain digestion of antibodies resulting in the production of two identical antigen-binding fragments, each with a single antigen-binding site and a residual “Fc” fragment. Pepsin treatment yields a F(ab′)2 fragment that has two antigen-combining sites capable of cross-linking antigen. An “Fv” is the minimum antibody fragment that contains a complete antigen recognition and binding site. In a two-chain Fv species, this region consists of a dimer of one heavy- and one light-chain variable domain in tight, non-covalent association. In a single-chain Fv (scFv) species, one heavy- and one light-chain variable domain are covalently linked by a flexible peptide linker such that the light and heavy chains associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although usually at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy-Chain C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Methods for producing the various fragments from monoclonal Abs include, e.g., Plückthun, 1992, Immunol. Rev: 130:152-188.

The term “monoclonal antibody” as used herein refers to an antibody obtained from a population of substantially homogeneous antibodies, i.e., the individual antibodies comprising the population are identical except for possible naturally occurring mutations that are present in minor amounts. In some embodiments monoclonal antibodies are made, for example, by the hybridoma method first described by Köhler and Milstein (1975) Nature 256:495, or are made by recombinant methods, e.g., as described in U.S. Pat. No. 4,816,567. In some embodiments monoclonal antibodies are isolated from phage antibody libraries using the techniques described in Clackson et al., Nature 352:624-628 (1991), as well as in Marks et al., J. Mol. Biol. 222:581-597 (1991).

As used herein, the term “epitope” refers to a fragment of a polypeptide or protein having antigenic or immunogenic activity in an animal, preferably in a mammal, and most preferably in a human. An epitope having immunogenic activity is a fragment of a polypeptide or protein that elicits an antibody response in an anima. An epitope having antigenic activity is a fragment of a polypeptide or protein to which an antibody immunospecifically binds as determined by any method, for example by immunoassays. Antigenic epitopes need not necessarily be immunogenic.

The phrase “specifically binds” when referring to the interaction between an antibody or other binding molecule and a protein or polypeptide or epitope, typically refers to an antibody or other binding molecule that recognizes and detectably binds with high affinity to the target of interest. Preferably, under designated or physiological conditions, the specified antibodies or binding molecules bind to a particular polypeptide, protein or epitope yet does not bind in a significant or undesirable amount to other molecules present in a sample. In other words the specified antibody or binding molecule does not undesirably cross-react, with non-target antigens and/or epitopes. Further, in some embodiments, an antibody that specifically binds, binds through the variable domain or the constant domain of the antibody. For the antibody that specifically binds through its variable domain, it is not aggregated, i.e., is monomeric. A variety of immunoassay formats are used to select antibodies or other binding molecule that are immunoreactive with a particular polypeptide and have a desired specificity. For example, solid-phase ELISA immunoassays, BIAcore, flow cytometry and radioimmunoassays are used to select monoclonal antibodies having a desired immunoreactivity and specificity. See, Harlow, 1988, ANTIBODIES, A LABORATORY MANUAL, Cold Spring Harbor Publications, New York (hereinafter, “Harlow”), for a description of immunoassay formats and conditions that are used to determine or assess immunoreactivity and specificity. “Selective binding”, “selectivity”, and the like refer the preference of a antibody to interact with one molecule as compared to another. Preferably, interactions between antibodies, particularly modulators, and proteins are both specific and selective. Note that in some embodiments a small antibody is designed to “specifically bind” and “selectively bind” two distinct, yet similar targets without binding to other undesirable targets.

### Cardiovascular Disorders

In some embodiments, the methods and compositions described herein treat a cardiovascular disorder. As used herein, the term “cardiovascular disease” (CVD) refers to a disease or disorder characterized by impairment or dysfunction of the heart, an artery, and/or vein. In some embodiments, the disorder is a dyslipidemia. In some embodiments, the disorder is hyperlipidemia; hypercholesterolemia; hyperglyceridemia; combined hyperlipidemia; hypolipoproteinemia; hypocholesterolemia; abetlipoproteinemia; Tangier disease; or a combination thereof. In some embodiments, the disorder is acute coronary syndrome; unstable angina; non-ST segment elevation myocardial infarction; ST segment elevation myocardial infarction; stable angina; Prinzmetal's angina; arteriosclerosis; atherosclerosis; arteriolosclerosis; stenosis; restenosis; venous thrombosis; arterial thrombosis; stroke; transient ischemic attack; peripheral vascular disease; coronary artery disease; obesity; diabetes; metabolic syndrome; or combinations thereof.

### Lipids and Lipoproteins

In some embodiments, the methods and compositions described herein treat dyslipidemia. As used herein, the term “dyslipidemia” means a disruption (i.e., variation from a normal range) in the concentration of a lipid in the blood.

In certain instances, a dyslipidemia is an increase in lipid (e.g. cholesterol, glycerides, or triglyceride) concentrations over a normal range (i.e., a hyperlipidemia). In certain instances, a hyperlipidemia involves an increase in the concentration of cholesterol (i.e., hypercholesterolemia); glycerides (i.e., hyperglyceridemia); triglycerides (i.e., hypertriglyceridemia); lipoproteins (i.e., hyperlipoproteinemia); chylomicrons (i.e., hyperchylomicronemia); or combinations thereof (e.g., combined hyperlipidemia). In certain instances, a dyslipidemia is a decrease in lipid concentrations below a normal range (i.e., a hypolipidemia). In certain instances, a hypolipidemia involves a decrease in the concentration of lipoproteins (i.e., hypolipoproteinemia); cholesterol (i.e., hypocholesterolemia); beta lipoproteins (i.e., abetalipoproteinemia); HDL (i.e., Tangier disease); or combinations thereof. In certain instances, a dyslipidemia results from environmental factors (e.g., lack of exercise or food intake). In certain instances, a dyslipidemia results from genetic factors (e.g., aberrant expression of ApoA1, Apo B, ApoC2, LPL, or LDL receptor).

In certain instances, blood comprises lipoproteins. In certain instances, a lipoprotein is a complex of proteins (e.g., ApoA1, ApoA2, ApoA4, ApoA5, ApoC1, ApoC2, ApoC3, ApoD, ApoE, LCAT, PAF-AN, PON1, GPX, serum amyloid A, α-1 antitrypsin, and amyloid-β) and lipids. In certain instances, a lipoprotein is a high density lipoprotein (HDL). In certain instances, a lipoprotein is a low density lipoprotein (LDL).

### HDL

HDL is a type of lipoprotein that transports cholesterol and triglycerides to the liver. In certain instances, HDL comprises ApoA1 and ApoA2. In certain instances, ApoA1 and ApoA2 are expressed in the liver. In certain instances, the liver synthesized HDL.

In certain instances, HDL transport cholesterol from cells to the liver, adrenals, ovary and/or testes. In certain instances, cholesterol transported to the liver is excreted as bile. In certain instances, cholesterol transported to adrenals, ovaries and/or testes are used to synthesize steroid hormones.

HDL comprises multiple sub-classes of lipoprotein. In certain instances; the subclasses of HDL differ in size, density, protein and lipid composition. In certain instances, some HDL are protective, anti-oxidative, anti-inflammatory and/or anti-atherogenic. In certain instances, some HDL are neutral. In certain instances, some HDL enhance oxidation, increase inflammation and/or are pro-atherogenic.

In certain instances, increasing the concentration of HDL across all or most sub-classes results in the production of reactive oxygen species (ROS). In certain instances, an enzyme associated with HDL modifies a phospholipid into an oxidized phospholipid. In certain instances, an enzyme associated with HDL modifies cholesterol into an oxidized sterol. In certain instances, an oxidized sterol and/or an oxidized phospholipid results in pro-inflammatory and/or pro-atherogenic HDL.

In certain instances, cholesteryl ester transfer protein (CETP) exchanges triglycerides transported by VLDL (very low density lipoprotein) for cholesteryl esters transported by HDL. In certain instances, the exchange of triglycerides for cholesteryl esters results in VLDL being processed into LDL. In certain instances, LDL is removed from circulation by the LDL receptor pathway. In certain instances, the triglycerides are degraded by hepatic lipase. In certain instances, delipidified HDL recirculate in the blood and transport additional lipids to the liver.

In certain instances, inhibiting CETP disrupts the metabolism of HDL. In certain instances, inhibiting CETP prevents transfer of HDL-cholesterol and increases circulating levels of cholesteryl-ester enriched (larger) HDL subfractions. In some embodiments, inhibiting (partially or fully) CETP treat CVD. In certain instances, slowing the catabolism of HDL increases total circulating HDL levels. In certain instances, increasing total circulating HDL levels treats atherogenesis. In some embodiments, inhibiting (partially or fully) CETP results (partially or fully) in inflammation and/or worsening of CVD. In certain instances, increasing total circulating HDL levels generates a lipid pool with reduced clearance (kinetics). In certain instances, reduced clearance of lipids increases HDL capacity to harbor oxidizable and potentially inflammatory lipid stores.

### LDL

Low-density lipoprotein (LDL) is a type of lipoprotein that transports cholesterol and triglycerides from the liver to peripheral tissues. In certain instances, LDL comprises an apolipoprotein B (ApoB). In certain instances, ApoB is expressed as two isoforms, ApoB48 and ApoB100. In certain instances, ApoB48 is synthesized by intestinal cells. In certain instances, ApoB100 is synthesized in the liver. In certain instances, Hsp110 stabilizes of ApoB.

### Cardiovascular Disorders

In some embodiments, the methods and compositions described herein treat atherosclerosis. As used herein, “atherosclerosis” means inflammation of an arterial wall. In certain instance, the inflammation results from (partially or fully) the accumulation of macrophage white blood cells. In certain instances, the inflammation results from (partially or fully) the presence of oxidized LDL. In certain instances, oxidized LDL damages an arterial wall. In certain instances, monocytes respond to (i.e., follow a chemotactic gradient to) the damaged arterial wall. In certain instances, the monocytes differentiate into macrophages. In certain instances, macrophages endocytose the oxidized-LDL (cells such as macrophages with endocytosed LDL are called “foam cells”). In certain instances, a foam cell dies. In certain instances, the rupture of a foam cell deposits oxidized cholesterol into the artery wall. In certain instances, the arterial wall becomes inflamed due to the damaged caused by the oxidized LDL. In certain instances, cells form a hard covering over the inflamed area. In certain instances, the cellular covering narrows an artery.

In certain instances, an atheromatous plaque is divided into three distinct components: (a) the atheroma (i.e., a nodular accumulation of a soft, flaky, yellowish material comprised of macrophages nearest the lumen of the artery; (b) areas of cholesterol crystals; and (c) calcification at the outer base.

In certain instances, an atherosclerotic plaque results (Partially or fully) in stenosis (i.e., the narrowing of blood vessel). In certain instances, stenosis results (partially or fully) in decreased blood flow. In some embodiments, the methods and compositions described herein treat stenosis and/or restenosis. In certain instances, an atherosclerotic plaque results (partially or fully) in the development of an aneurysm. In some embodiments, the methods and compositions described herein treat an aneurysm. In certain instances, the rupture of an atherosclerotic plaque results (partially or fully) in an infarction (i.e., the deprivation of oxygen) to a tissue. In some embodiments, the methods and compositions described herein treat an infarction.

In some embodiments, the methods and compositions described herein treat a myocardial infarction. “Myocardial infarction” and “heart attack” are used interchangeably. As used herein, both terms refer to an interruption in the blood supply to the heart. In certain instances, an interruption in the blood supply to the heart results from (partially or fully) the occlusion of a coronary artery by a ruptured atherosclerotic plaque. In certain instances, occlusion of an artery results in the infarction of myocardium. In certain instances, the infarction of myocardium results in the scarring of myocardial tissue. In certain instances, scarred of myocardial tissue conducts electrical impulses more slowly than unscarred tissue. In certain instances, the difference in conduction velocity between scarred and unscarred tissue results (partially or fully) in ventricular fibrillation or ventricular tachycardia.

In some embodiments, the methods and compositions described herein treat an angina (e.g., stable or unstable). As used herein, “angina pectoris” refers chest pain resulting from (partially or fully) of the heart.

In some embodiments, the methods and compositions described herein treat a thrombosis (venous or arterial). As used herein, “thrombosis” refers to the formation of a blood clot. In certain instances, the blood clot forms in a vein (i.e., venous thrombosis). In certain instances, the blood clot forms in an artery (i.e., arterial thrombosis). In certain instances, a piece of or the entire blood clot is transported (i.e., an embolism) to the lungs (i.e., a pulmonary embolism). In some embodiments, the methods and compositions described herein treat an embolism.

In some embodiments, the methods and compositions described herein treat a stroke. As used herein, “stroke” refers to a loss of brain function (e.g., necrosis of brain tissue) resulting from (partially or fully) a disturbance in blood supply (e.g., ischemia). In certain instances, a stroke results from (partially or fully) a thrombosis or an embolism.

In certain instances, an atherosclerotic plaque results (partially or fully) in the development of an aneurysm. In some embodiments, the methods and compositions described herein treat an aneurysm. In some embodiments, the methods and compositions described herein treat an abdominal aortic aneurysm (“AAA”). As used herein, an “abdominal aortic aneurysm” is a localized dilatation of the abdominal aorta. In certain instances, the rupture of an AAA results in bleeding, leading to hypovolemic shock with hypotension, tachycardia, cyanosis, and altered mental status.

In some embodiments, the compositions and methods disclosed herein treat abdominal aortic aneurysms. In certain instances, abdominal aortic aneurysms result (partially or fully) from an extensive breakdown of structural proteins (e.g., elastin and collagen). In some, embodiments, a method and/or composition disclosed herein partially or fully inhibits the breakdown of a structural protein (e.g., elastin and collagen). In certain instances, the breakdown of structural proteins is caused by activated MMPs. In some embodiments, a method and/or composition disclosed herein partially or fully inhibits the activation of an MMP. In some embodiments, a composition and/or method disclosed herein inhibit the upregulation of MMP-1, MMP-9 or MMP-12: In certain instances, MIF is co-expressed with MMP-1, MMP-9, and MMP-12 in abdominal aortic aneurysms. In certain instances, the MIF is upregulated in stable abdominal aortic aneurysm and is intensified further in ruptured aneurysms. In certain instances, MMPs are activated following infiltration of a section of the abdominal aorta by leukocytes (e.g., macrophages and neutrophils). In some embodiments, a method and/or composition disclosed herein partially or fully inhibits the activity of MIF. In some embodiments, a method and/or composition disclosed herein partially or fully inhibits the infiltration of a section of the abdominal aorta by leukocytes.

### Treatments for Cardiovascular Disorders

In some embodiments, the cardiovascular disorder is treated with an active agent (the “cardiovascular disorder agent”). In some embodiments, the active agent is niacin; a fibrate; a statin; an apolipoprotein A-1 modulator; an ACAT modulator; a CETP modulator; a glycoprotein modulator; a P2Y12 modulator; an Lp-PLA2 modulator; or combinations thereof.

In some embodiments, the Cardiovascular disorder agent reduces the risk of developing a cardiovascular disorder across all levels of HDL. In some embodiments, the cardiovascular disorder agent inhibits (partially or fully) the activity of 3-hydroxy-3-methylglutaryl coenzyme A reductase. In some embodiments, the cardiovascular disorder agent is a atorvastatin; cerivastatin; fluvastatin; lovastatin; mevastatin; pitavastatin; pravastatin; rosuvastatin; simvastatin; simvastatin and ezetimibe; lovastatin and niacin, extended-release; atorvastatin and amlodipine besylate; simvastatin and niacin, extended-release; or combinations thereof.

In some embodiments, the cardiovascular disorder agent raises HDL non-selectively. In some embodiments, the cardiovascular disorder agent down-regulates transcription of a CETP gene. In some embodiments, the cardiovascular disorder agent is niacin.

In some embodiments, the cardiovascular disorder agent reduces the risk of developing a cardiovascular disorder in individuals with low HDL with metabolic syndrome. In some embodiments, the cardiovascular disorder agent is bezafibrate; ciprofibrate; clofibrate; gemfibrozil; fenofibrate; or combinations thereof.

In some embodiments, the cardiovascular disorder agent selectively increases the levels of apoA1 protein (e.g. by transcriptional induction of the gene encoding apoA1) and increases the production of nascent. HDL (apoA1-enriched). In some embodiments, the second active agent is DF4 (Ac-D-W-F-K-A-F-Y-D-K-V-AA-E-K-F-K-E-A-F-NH2); DF5; RVX-208 (Resverlogix); or combinations thereof.

In some embodiments, the cardiovascular disorder agent inhibits the activity of Acyl-CoA cholesteryl acyl transferase (ACAT). In some embodiments, the cardiovascular disorder agent inhibits (partially or fully) the formation of foam cells and the accumulation of cholesterol esters in macrophages and vascular tissue. In some embodiments, the second active agent is avasimibe; pactimibe sulfate (CS-505); CI-1011 (2,6-diisopropylphenyl [(2,4,6-triisopropylphenyl)acetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N [[4-(2,2-dimethylpropyl)phenyl]methyl]-N-(hepthyl)urea); F-1394 ((1s,2s)-2-[3-(2,2-dimethylpropyl)-3-nonylureido]aminocyclohexane-1-yl-3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; or combinations thereof.

In some embodiments, the cardiovascular disorder agent inhibits (partially or completely) the activity of Cholesteryl Ester Transfer Protein (CETP). In some embodiments, the cardiovascular disorder agent increases HDL-C concentration and reduces LDL-C concentration. In some embodiments, the cardiovascular disorder agent increases antioxidant enzymes associated with HDL and decreases oxidized LDL. In some embodiments, the cardiovascular disorder agent is torcetrapib; anacetrapid; JTT-705 (Japan Tobacco/Roche); or combinations thereof.

In some embodiments, the cardiovascular disorder agent inhibits (partially or fully) the activity of glycoprotein IIb/IIIa. In some embodiments, the cardiovascular disorder agent prevents (partially or fully) platelet aggregation and/or thrombus formation. In some, embodiments, the cardiovascular disorder agent is abciximab; eptifibatide; tirofiban; roxifiban; variabilin; XV 459 (N(3)-(2-(3-(4-formamidinophenyl)isoxazolin-5-yl)acetyl)-N(2)-(1-butyloxycarbonyl)-2,3-diaminopropionate); SR 121566A (3-[N-{-4-[4-(aminoiminomethyl)phenyl]-1,3-thiazol-2-yl}-N-(1-carboxymethylpiperid-4-yl)amino]propionic acid, trihydrochloride); FK419 ((S)-2-acetylamino-3-[(R)-[1-[3-(piperidin-4-yl) propionyl]piperidin-3-ylcarbonyl]amino]propionic acid trihydrate); or combinations thereof.

In some embodiments, the cardiovascular disorder agent antagonizes P2Y12. In some embodiments, the cardiovascular disorder agent inhibits (partially or fully) platelet aggregation. In some embodiments, the cardiovascular disorder agent is clopidogrel; prasugrel; cangrelor; AZD6140 (AstraZeneca); MRS 2395 (2,2-Dimethyl-propionic acid 3-(2-chloro-6-methylaminopurin-9-yl)-dimethyl-propionyloxymethyl)-propyl ester); BX 667 (Berlex Biosciences); BX 048 (Berlex Biosciences) or combinations thereof

In some embodiments, the cardiovascular disorder agent inhibits (partially or fully) the activity of lipoprotein-associated phospholipase A2 (1p-PLA2). In some embodiments, the cardiovascular disorder agent inhibits (partially of fully) the hydrolysis of the center (sn-2) ester bond of phospholipids. In some embodiments, the cardiovascular disorder agent inhibits (partially or fully) the production of oxidized fatty acids and lysophosphatidyl choline. In some embodiments, the cardiovascular disorder agent inhibits (partially or fully) the chemotaxis of monocytes. In some embodiments, the cardiovascular disorder agent is darapladib (SB 480848); SB-435-495 (GlaxoSmithKline); SB-222657 (GlaxoSmithKline); SB-253514 (GlaxoSmithKline); or combinations thereof.

In some embodiments, the cardiovascular disorder agent inhibits a leukotriene (e.g., by antagonizing LTA4, LTB4, LTC4, LTD4, LTE4, LTF4, LTA4R; LTB4R; LTB4R1, LTB4R2, LTC4R, LTD4R, LTE4R, CYSLTR1, or CYSLTR2; or by inhibiting the Synthesis of a leukotriene via 5-LO, FLAP, LTA4H, LTA4S, or LTC4S). In some embodiments, the second active agent is an antagonist of 5-LO. In some embodiments, the second active agent is an antagonist of FLAP. In some embodiments, the second active agent is A-81834 (3-(3-(1,1-dimethylethylthio-5-(quinoline-2-ylmethoxy)-1-(4-chloromethylphenyl)indole-2-yl)-2,2-dimethylpropionaldehydro oxime-O-2-acetic acid; AME103 (Amira); AME803 (Amira); atreleuton; BAY-x-1005 ((R)-(+)-alpha-cyclopentyl-4-(2-quinolinylinethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)-S-[[4-(dimethylamino)phenyl]methyl]-N-(3-mercapto-2-methyl-1-oxopropyl-L-cycteine); SC-56938 (ethyl-1- [2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4-yl)phenoxy)methyl)-1-Methyl-2(1H)-quinlolinone); or combinations thereof.

In some embodiments, the cardiovascular disorder agent is administered before, after, or simultaneously with the modulator of inflammation.

In some embodiments, a cardiovascular disorder is treated by delipidifying the blood of an individual. In some embodiments, the blood of an individual is delipidified by removing a lipid from an HDL molecule in an individual in need thereof. In some embodiments, administering a therapeutically-effective amount of a modulator of inflammation acts in synergy with the removal of a lipid from an HDL molecule.

### III. Macrophage Migration Inhibitory Factor (MIF)

In some embodiments, the methods and compositions disclosed herein inhibit (partially or fully) the activity of MIF. MIF is a pro-inflammatory lymphokine. In certain instances, it is secreted by a lymphocyte (e.g. a T-cell) in response to an infection, inflammation, or tissue injury. In certain instances, MIF is a functional noncognate ligand for the receptors CXCR2 and CXCR4. In some embodiments, the methods and compositions disclosed herein inhibit (partially or fully) the activity of CXCR2 and/or CXCR4.

In certain instances, MIF induces chemotaxis in nearby: leukocytes (e.g. lymphocytes, granulocytes and monocytes/macrophages) along a MIF gradient. In certain instances, MIF induces the chemotaxis of a leukocyte (e.g. lymphocytes, granulocytes and monocytes/macrophages) to the site of an infection, inflammation or tissue injury. In certain instances, the chemotaxis of a leukocyte (e.g. lymphocytes, granulocytes and monocytes/macrophages) along a MIF gradient results in inflammation at the site of infection, inflammation, or tissue injury. In certain instances, the chemotaxis of monocytes along a RANTES gradient results in monocyte arrest (i.e., the deposition of monocytes on epithelium) at the site of injury or inflammation.

In certain instances, a human MIF polypeptide is encoded by a nucleotide sequence located on chromosome 22 at the cytogenic band 22q11.23. In certain instances, a MIF protein is a 12.3 kDa protein. In certain instances, a MIF protein is a homotrimer comprising three polypeptides of 115 amino acids. In certain instances, a MIF protein comprises a pseudo-ELR motif that mimics the ELR motif found in chemokines. In certain instances, a pseudo-ELR motif of a MIF protein mediates binding to a CXCR2 and/or CXCR4 receptor. In certain instances, a MIF protein comprises a 10- to 20-residue N-terminal Loop motif (N-loop). In certain, instances, a MIF N-loop mediates binding to a CXCR2 and/or CXCR4 receptor.

In some embodiments, the methods, described herein comprise a CXCR2 antagonist; an anti-CXCR2 antibody; a CXCR4 antagonist; an anti-CXCR4 antibody; a MIF antagonist (e.g., a peptide, polypeptide, or small molecule); an anti-MIF antibody; or combinations thereof. In some embodiments, the antagonist inhibits the binding of MIF to CXCR2 and/or CXCR4 by binding to a pseudo-ELR motif of MIF. In some embodiments, the antagonist inhibits the binding of MIF to CXCR2 and/or CXCR4 by binding to an N-loop motif of MIF.

### A. Disruption of MIF Domains

In some embodiments, the modulator of MIF disrupts the ability of MIF to interact with CXCR2, CXCR4, CD74, or a combination thereof. In some embodiments, the ability of MIF to interact with CXCR2, CXCR4, CD74, or a combination thereof is inhibited by occupying, masking, or otherwise disrupting domains on MIF to which CXCR2, CXCR4, and/or CD74 bind (e.g., the N-loop and/or the pseudo-ELR loop).

In some embodiments, the ability of MIF to interact with CXCR2, CXCR4, CD74, or a combination thereof is inhibited by a small molecule, peptide, antibody, and/or peptibody occupying, masking, or otherwise disrupting domains on MIF to which CXCR2, CXCR4, and/or CD74 bind. In some embodiments, a small molecule, peptide, antibody, and/or peptibody inhibits MIF binding to CXCR2, CXCR4, and/or CD74. In certain instances, occupying, masking, or otherwise disrupting domains on MIF does not affect CXCR2 and CXCR4 signaling mediated by other agonists/ligands (e.g., IL-8/CXCL8, GRObeta/CXCL2 and/or Stromal Cell-Derived. Factor-1a (SDF-1a)/CXCL12).

In certain instances, the pseudo-ELR region of MIF mediates ligand (e.g., CD74, CXCR2, CXCR4) binding to MIF. In some embodiments, the binding of a small molecule, peptide, antibody, and/or peptibody to a pseudo-ELR loop of MIF inhibits the ability of MIF to form a signaling complex with CXCR2, CXCR4, CD74, or a Combination thereof. In some embodiments, the binding of a small molecule, peptide, antibody, and/or peptibody to a pseudo-ELR loop of MIF invokes a conformational change in MIF that prevents receptor or substrate interactions.

In certain instances, the N-loop region of MIF mediates ligand (e.g., CD74, CXCR2, CXCR4) binding to MIF, In some embodiments; the binding of a small molecule, peptide, antibody, and/or peptibody to an N-loop motif of MIF inhibits the ability of MIF to form a signaling complex with CXCR2, CXCR4, CD74, or a combination thereof. In some embodiments, the binding of a small molecule, peptide, antibody, and/or peptibody to an N-loop motif of MIF invokes a conformational change in MIF that prevents receptor or substrate interactions.

In certain instances, amino acids 65-94 of MIF (e.g., IGKIGGAQNRSYSKLLCGLLAERLRISPDR; numbering includes the first methionine) mediate CXCR2 binding to MIF. In some embodiments, the binding of a small molecule, peptide, antibody, and/or peptibody to amino acids 65-94 of MIF inhibits the ability of MIF to form a signaling complex with CXCR2. In some embodiments, the binding of a peptide to amino acids 65-94 of MIF inhibits the ability of MIF to form a signaling complex with CXCR2. In some embodiments, the binding of an antibody to amino acids 65-940f MIF inhibits the ability of MIF to form a signaling complex with CXCR2. In some embodiments, the binding of a peptibody to, amino acids 65-94 of MIF inhibits the ability of MIF to form a signaling complex with CXCR2. In some embodiments, the binding of a small molecule to amino acids 65-94 of MIF inhibits the ability of MIF to form a signaling complex with CXCR2.

In certain instances, amino acids 80-95 of MIF (e.g., LCGLLAERLRISPDRV; numbering includes the first methionine) mediate ligand binding to MIF. In some embodiments, the binding of a small molecule, peptide, antibody, and/or peptibody to amino acids 80-95 of MIF inhibits the ability of MIF to form a signaling complex with a ligand. In some embodiments, the binding of a peptide to amino acids 80-95 of MIF inhibits the ability of MIF to form a signaling complex with a ligand. In some embodiments, the binding of an antibody to amino acids 80-95 of MIF inhibits the ability of MIF to form a signaling complex with a ligand. In some embodiments, the binding of a peptibody to amino acids 80-95 of MIF inhibits the ability of MW to form a signaling complex with a ligand. In some embodiments, the binding: of a small molecule to amino acids 80-95 of MIF inhibits the ability of MIF to form a signaling complex with a ligand.

In some embodiments, the modulator of MO, is a peptide that occupies, masks, or otherwise disrupts a domain on MIF to which CXCR2, CXCR4, and/or CD74 binds. In some embodiments, the peptide specifically binds to all or a portion of the pseudo-ELR loop of MIF. In some embodiments, the peptide specifically binds to all or a portion of the N-loop motif of MIF. In some embodiments, the peptide specifically binds to all or a portion of both the pseudo-ELR and N-loop motifs.

In some embodiments, the modulator of MIF is a peptide that specifically binds to all or a portion of a peptide sequence as follows: VNTNVPPRASVPDGFLSELTQQLAQATGKPPQYIAVHVVPDQL and the corresponding feature/domain of at least one of a MIF monomer or MIF timer; a peptide that specifically binds to all or a portion of a peptide sequence as follows: PDQLMAFGGSSEPCALCSL and the corresponding feature/domain of at least one of a MIF monomer or MIF trimer; a peptide that specifically binds to all or a portion of a peptide sequence as follows: VNTNVPPRASVPDGFLSELTQQLAQATGKPPQYIAVHVVPDQLMAFGGSSEPCALCSL and the corresponding feature/domain of at least one of a MIF monomer or MIF trimer; a peptide that specifically binds to all or a portion of a peptide sequence as follows: PDQLMAFGGSSEPCALCSLHSI and the corresponding feature/domain of at least one of a MIF monomer or MIF trimer; or combinations thereof.

In some embodiments, the modulator of MIF is an antibody that occupies, masks, or otherwise disrupts a domain on MIF to which CXCR2, CXCR4, and/or CD74 binds. In some embodiments, the antibody specifically binds to all or a portion of the pseudo-ELR loop of MIF. In some embodiments, the antibody specifically binds to all or a portion of the N-loop motif of MIF. In some embodiments, the antibody specifically binds to all or a portion of both the pseudo-ELR and N-loop motifs.

In some embodiments, the modulator of MIF is a peptibody that occupies, masks, or otherwise disrupts a domain on MIF to which CXCR2, CXCR4, and/or CD74 binds. In some embodiments, the peptibody specifically binds to all or a portion of the pseudo-ELR loop of MIF. In some embodiments, the peptibody specifically binds to all or a portion of the N-loop motif of MIF. In some embodiments, the peptibody specifically binds to all or a portion of both the pseudo-ELR and N-loop motifs.

In some embodiments, the modulator of MIF is a small molecule that occupies, masks, or otherwise disrupts a domain on MIF to which CXCR2, CXCR4, and/or CD74 binds. In some embodiments, the small molecule specifically binds to all or a portion of the pseudo-ELR loop of MIF. In some embodiments, the small molecule specifically binds to all or a portion of the N-loop motif of MIF. In some embodiments, the small; molecule specifically binds to all or a portion of both the pseudo-ELR and N-loop motifs.

### B. Disruption of CXCR2 and CXCR4Domains

In some embodiments, the modulator of MIF is an agent that occupies; masks, or otherwise disrupts a domain on CXCR2 and/or CXCR4 to which MIF and/or CD74 bind. In some embodiments, the modulator of MIF is an agent that disrupts the ability of MIF to form a signaling complex with CXCR2, CXCR4, CD74, or a combination thereof.

In some embodiments, the agent that inhibits the binding of MIF and/or CD74 to CXCR2 and/or CXCR4 is a peptide.

In some embodiments, the agent that inhibits the binding of MIF and/or CD74 to CXCR2 and/or CXCR4 is an antibody.

In some embodiments, the agent that inhibits the binding of MIF and/or CD74 to CXCR2 and/or CXCR4 is a peptibody.

In some embodiments, the agent that inhibits the binding of MIF and/or CD74 to CXCR2 and/or CXCR4 is a derivative of hydroxycinnamate, Schiff-based tryptophan analogs, or imino-quinone metabolites of acetaminophen.

In some embodiments, the agent that inhibits the binding of MIF and/or CD74 to CXCR and/or CXCR4 is glyburide, probenicide, DIDS (4,4-diisothiocyanatostilbene-2,2-disulfonic acid), bumetanide, furosemide, sulfobromophthalein, diphenylamine-2-carboxylic acid, flufenamic acid, or combinations thereof.

In some embodiments, the agent that inhibits the binding of MIF and/or CD74 to CXCR2 is CXCL8₍₃₋₇₄₎K11R/G31P; IL-8₍₄₋₇₂₎; IL-8₍₆₋₇₂₎; recombinant IL-8 (rIL-8); recombinant IL-8, NMeLeu (rhIL-8 with an N-methylated leucine at position 25); (AAR)IL-8 (IL-8 with N-terminal Ala-4-Ala5 instead of Glu4-Leu5); GRO-alpha₍₁₋₇₃₎ (also known as CXCL1); GRO-alpha₍₄₋₇₃₎; GRO-alpha₍₅₋₇₃₎; GRO-alpha₍₆₋₇₃₎; recombinant GRO (rGRO); (ELR)PF4 (PF4 with an ELR seq at the N-terminus); recombinant PF4 (rPF4); Antileukinate; Sch527123 (-hydroxy-N,N-dimethyl-3-{2-[[(R)-1-(5-methyl-furan-2-yl)-propyl]amino]-3,4-dioxo-cyclobut-1-enylamino}-benzamide); N-(3-(aminosulfonyl)-4-chloro-2-hydroxyphenyl)-N′-(2,3-dichlorophenyl)urea; SB-517785-M (GSK); SB 265610 (N-(2-Bromophenyl)-N′-(7-cyano-1H-benzotriazol-4-yl)urea); SB225002 (N-(2-Brothophenyl)-N′-(2-hydroxy-4-nitrophenyl)urea); SB455821 (GSK), SB272844 (GSK); DF2162 (4-[(1R)-2-amino-1-methyl-2-oxoethyl]phenyl trifluoromethanesulphonate); Reparixin; or combinations thereof.

In some embodiments, the agent that inhibits the binding of MIF and/or CD74 to CXCR4 is ALX40-4C(N-alpha-acetyl-nona-D-arginine amide acetate); AMD-070 (AMD11070, AnorMED); Plerixafor (AMD3100); AMD3465(AnorMED); AMD8664 (1-pyridin-2-yl-N4-[-(1,4,7-triazacyclotetradecan-4-ylmethyl)benzyl]methanamine); KRH-1636 (Kureha Chemical Industry Co. Limited); KRH-2731 (Kureha Chemical Industry Co. Limited); KRH-3955 (Kureha Chemical Industry Co. Limited); KRH-3140 (Kureha Chemical Industry Co. Limited); T134 (L-citrulline-16-TW70 substituted for the C-terminal amide by a carboxylic acid); T22 ([Tyr^{5,12}, Lys⁷]-polyphemusin II); TW70 (des-[Cys8,13, Tyr9,12]-[D-Lys10, Pro11]-T22); T140 (H-Arg-Arg-NaI-Cys-Tyr-Arg-Lys7D-Lys-Pro-Tyr-Arg-Cit-Cys-Arg-OH); TC14012 (R-R-Nal-C-Y-(L)Cit-K-(D)Cit-P-Y-R-(L)citrulline-C—R—NH2, where Nal=L-3-(2-naphthylalanine), Cit=citruline and the peptide is cyclized with the cysteines); TN14003; RCP168 (νMIP-II₍₁₁₋₇₁₎ with D-amino acids added to the N terminus); POL3026 (Arg(*)-Arg-NaI(2)-Cys(1×)-Tyr-Gln-Lys-(d-Pro)-Pro-Tyr-Arg-Cit-Cys(1×)-Arg-Gly-(d-Pro)(*)); POL2438; compound 3 (N-(1-methyl-1-phenylethyl)-N-[((3S)-1-{2-[5-(4H-1,2,4-triazol-4-yl)-1H-indol-3-yl]ethyl}pyrrolidin-3-yl)methyl]amine); isothioureas 1a-1u (for information regarding isothioureas 1a-1u see Gebhard Thoma, et al., Orally Bioavailable Isothioureas Block Function of the Chemokine Receptor CXCR4 In Vitro and In Vivo, J. Med. Chem., Article ASAP (2008), which is herein incorporated by reference for such disclosures); or combinations thereof.

In some embodiments, the agent that inhibits the binding of MIF and/or CD74 to CXCR2 and/or CXCR4 is MIF is COR100140 (Genzyme Corp/Cortical Pty Ltd.); ISO-1 ((S,R)-3-(4-Hydroxyphenyl)-4,5-dihydro-5-isoxazole acetic acid, methyl ester); 4-IPP (4-iodo-6-phenylpyrimidine); or combinations thereof.,

### C. Disruption of CD74 Domains

In some embodiments; the modulator of MIF is an agent that occupies, masks, or otherwise disrupts a domain on CD74 to which MIF, CXCR2, and/or CXCR4 bind. In some embodiments, the modulator of MIF is an agent that disrupts the ability of MIF to form a signaling complex with CXCR2, CXCR4, CD74, or a combination thereof.

In some embodiments, the agent that inhibits the binding of MIF, CXCR2, CXCR4, or a combination thereof to CD74 is a peptide.

In some embodiments, the agent that inhibits the binding of MIF, CXCR2, CXCR4, or a combination thereof to CD74 is an antibody. In some embodiments, the agent that inhibits the binding of MIF, CXCR2, CXCR4, or a combination thereof to CD74 is M-B741, 555538 (BD Pharmingen).

In some embodiments, the agent that inhibits the binding of MIF, CXCR2, CXCR4, or a combination thereof to CD74 is a peptibody.

In some embodiments, the agent that inhibits the binding of MIF, CXCR2, CXCR4, or a combination thereof to CD74 is a small molecule.

In certain instances, occupying, masking, or otherwise disrupting domains on MIF does not affect CD74 signaling mediated by other agonists/ligands (e.g., IL-8/CXCL8, GRObeta/CXCL2 and/or Stromal Cell-Derived Factor-1a (SDF-1a)/CXCL12).

### D. MIF Mimics

In some embodiments, the modulator of MIF is an agent that disrupts the ability of MIF to form a signaling complex with CXCR2, CXCR4, CD74, or a combination thereof. In some embodiments, the modulator of MIF is a MIF-like peptide that mimics part or all of a MIF domain (e.g., the pseudo-ELR, or N-Loop domains). In some embodiments, the MIF-mimic binds to CXCR2, CXCR4, CD74, or a combination thereof and thus prevents CXCR2, CXCR4, or CD74 from binding to MIF.

In some embodiments, the MIF-Mimic adopts structural or functional features similar to the N-Loop motif of MIF. In some embodiments, the MIF-mimic is a peptide. In some embodiments, the MIF-mimic comprises a peptide of Formula (I):

X¹-X²-Q/A-X³-X⁴-X⁵-X⁶-G/S-X⁷-X⁸-X⁹-X-P-X¹¹

wherein:
X¹ is selected from the group consisting of threonine, glycine, proline and alanine;
X² is selected from the group consisting of glycine, asparagine, aspartic acid, and serine;
X³ is selected from the group consisting of methionine, isoleucine, leucine, alanine, proline, lysine, glutamine, arginine and lysine;
X⁴ is selected from the group consisting of methionine, isoleucine and leucine;
X⁵ is selected from the group consisting of alanine, threonine, methionine, serine and valine;
X⁶ is selected from the group consisting of phenylalanine, histidine, arginine and lysine;
X⁷ is selected from the group consisting of aspartic acid, glutamic acid, threonine, glycine and alanine;
X⁸ is selected from the group consisting of serine, threonine, lysine and arginine;
X⁹ is selected from the group consisting of serine, asparagine, glycine, threonine, aspartic acid, glutamic acid, glutamine and histidine;
X¹⁰ is selected from the group consisting of aspartic acid, glutamic acid, alanine and asparagine; and
X¹¹ is selected from the group consisting of cysteine, alanine, serine, threonine and valine.

In some embodiments, X¹ is proline. In some embodiments, X² is aspartic acid. In some embodiments, X³ is leucine. In some embodiments, X⁴ is methionine. In some embodiments, X⁵ is alanine. In some embodiments, X⁶ is phenylalanine. In sortie embodiments, X⁷ is glycine. In some embodiments, X⁸ is serine. In some embodiments, X⁹ is serine. In some embodiments, X¹⁰ is glutamic acid. In some embodiments, X¹¹ is serine cysteine.

In some embodiments, the MIF-mimic comprises any 5 or more consecutive peptide of Formula (I).

In some embodiments, the MIF-mimic comprises 5 or more consecutive amino acids of human MIF₄₄₋₅₇ (numbering includes the first methionine). In some embodiments, the MIF-mimic comprises 5 or more consecutive amino acids of murine. MIF₄₄₋₅₇. In some embodiments, the MIF-mimic comprises 5 or more consecutive amino acids of porcine MIF₄₄₋₅₇. In some embodiments, the MIF-mimic comprises 5 or more consecutive amino acids of bovine MIF₄₄₋₅₇. In some embodiments, the MIF-mimic comprises 5 or more consecutive amino acids of rat MIF₄₄₋₅₇.

In some embodiments, the MIF-mimic comprises one or more of the peptides selected from Table 1. In Some embodiments, the MIF-mimic comprises N- and/or C-terminal chemical modifications to improve ADME-PK. In some embodiments, MIF-mimic comprises non-natural amino acids. In some embodiments, MIF-mimic comprises cyclical variants.

TABLE 1

| | | |
|---|---|---|
| LMAFGGSSEPCALC | SEPCAL | cyclo |
| | | (GSSEPCALC) |
| LMAFGGSSEPCAL | EPCALC | cyclo |
| | | (GSSEPCAL) |
| LMAFGGSSEPCA | QLMAFGGSSEPCALC | cyclo |
| | | (GSSEPCA) |
| LMAFGGSSEPC | QLMAFGGSSEPCAL | cyclo |
| | | (GSSEPC) |
| LMAFGGSSEP | QLMAFGGSSEPCA | cyclo |
| | | (SSEPCALC) |
| LMAFGGSSE | QLMAFGGSSEPC | cyclo |
| | | (SSEPCAL) |
| LMAFGGSS | QLMAFGGSSEP | cyclo |
| | | (SSEPCA) |
| LMAFGGS | QLMAFGGSSE | cyclo |
| | | (SEPCALC) |
| LMAFGG | QLMAFGGSS | cyclo |
| | | (SEPCAL) |
| MAFGGSSEPCALC | QLMAFGGS | cyclo |
| | | (EPCALC) |
| MAFGGSSEPCAL | QLMAFGG | cyclo |
| | | (QLMAFGGSSEPCALC) |
| MAFGGSSEPCA | QLMAFG | cyclo |
| | | (QLMAFGGSSEPCAL) |
| MAFGGSSEPC | CSSEPCALC(1096) | cyclo |
| | | (QLMAFGGSSEPCA) |
| MAFGGSSEP | CFGGSSEPCALC | cyclo |
| | (1081) | (QLMAFGGSSEPC) |
| MAFGGSSE | CLMAFGGSSEPCALC | cyclo |
| | (1057) | (QLMAFGGSSEP) |
| MAFGGSS | CAFGGSSC(1079) | cyclo |
| | | (QLMAFGGSSE) |
| MAFGGS | CLMAFGGSSEPC C | cyclo |
| | (1059) | (QLMAFGGSS) |
| AFGGSSEPCALC | CAFGGSSEPCAC | cyclo |
| | (1075) | (QLMAFGGS) |
| AFGGSSEPCAL | CMAFGGSSEPC | cyclo |
| | | (QLMAFGG) |
| AFGGSSEPCA | CGGSSEPCAC | cyclo |
| | | (QLMAFG) |
| AFGGSSEPC | NVPRASVPD | cyclo |
| | | (AFGGSSEPCALC) |
| AFGGSSEP | VPDGFLSEL | cyclo |
| | | (AFGGSSEPCAL) |
| AFGGSSE | CFGGSSEPC | cyclo |
| | | (AFGGSSEPCA) |
| AFGGSS | IAVHVVPDQLMAFGG | cyclo |
| | SSEPC | (AFGGSSEPC) |
| FGGSSEPCALC | CLHSIGKIGGAQNRS | cyclo |
| | YSKLL | (AFGGSSEP) |
| FGGSSEPCAL | PCALLCSLHSIGKIG | cyclo |
| | | (AFGGSSE) |
| FGGSSEPCA | CSLHSIGKIGGAQNR | cyclo |
| | | (AFGGSS) |
| FGGSSEPC | IGKIGGAQNRSYSKL | cyclo |
| | | (FGGSSEPCALC) |
| FGGSSEP | GAQNRSYSKLLCGLLA | cyclo |
| | | (FGGSSEPCAL) |
| FGGSSE | CGLLAERLRISPDRV | cyclo |
| | | (FGGSSEPCA) |
| GGSSEPCALC | ERLRISPDRVYINYY | cyclo |
| | | (FGGSSEPC) |
| GGSSEPCAL | cyclo | cyclo |
| | (LMAFGGSSEPCALC) | (FGGSSEP) |
| GGSSEPCA | cyclo | cyolo |
| | (LMAFGGSSEPCAL) | (FGGSSE) |
| GGSSEPC | cyclo | cyclo |
| | (LMAFGGSSEPCA) | (GGSSEPCALC) |
| GGSSEP | cyclo | cyclo |
| | (LMAFGGSSEPC) | (GGSSEPCAL) |
| GSSEPCALC | cyclo | cyclo |
| | (LMAFGGSSEP) | (GGSSEPCA) |
| GSSEPCAL | cyclo | cyclo |
| | (LMAFGGSSE) | (GGSSEPC) |
| GSSEPCA | cyclo | cyclo |
| | (LMAFGGSS) | (GGSSEP) |
| GSSEPC | cyclo | cyclo |
| | (LMAFGGS) | (CSSEPCALC) |
| SSEPCALC | cyclo | cyclo |
| | (LMAFGG) | (CFGGSSEPCALC) |
| GSSEPCALC | cyclo | cyclo |
| | (MAFGGSSEPCALC) | (CFGGSSEPCC) |
| GSSEPCAL | cyclo | cyclo |
| | (MAFGGSSEPCAL) | (CFGGSSEPC) |
| GSSEPCA | cyclo | cyclo |
| | (MAFGGSSEPCA) | (CGSSEPCALC) |
| GSSEPC | cyclo | cyclo |
| | (MAFGGSSEPC) | (CAFGGSSEPCAC) |
| SSEPCALC | cyclo | cyclo |
| | (MAFGGSSEP) | (CLMAFGGSSEPCALC) |
| SSEPCAL | cyclo | cyclo |
| | (MAFGGSSE) | (CAFGGSSC) |
| SSEPCA | cyclo | |
| | (MAFGGSS) | |
| SEPCALC | cyclo | |
| | (MAFGGS) | |

In some embodiments, the MIF-mimic adopts structural or functional features similar to the pseudo-ELR loop of MIF. In some embodiments, the MIF-mimic is a peptide.

In some embodiments, the MIF-mimic comprises a peptide of Formula (II):

X¹-X²-T/S—N—X³-X⁴-X⁵-X⁶-X⁷-X⁸-P/S—X⁹-X¹⁰

wherein:
X¹ is selected from the group consisting of valine, isoleucine, threonine, phenylalanine and leucine;
X² is selected from the group asparagine, arginine, aspartic acid, glutamic acid, serine and alanine;
X³ is selected from the group valine, isoleucine, arginine, lysine and leucine;
X⁴ is selected from the group proline, alanine, cysteine and leucine;
X⁵ is selected from the group arginine, lysine, glutamine, serine, alanine, aspartic acid, glutamic acid and asparagine;
X⁶ is selected from the group alanine, aspartic acid, glutamic acid, asparagine, serine and glutamine;
X⁷ is selected from the group serine, glutamic acid, aspartic acid, asparagine, arginine, glycine, lysine and arginine;
X⁸ is selected from the group valine, isoleucine and phenylalanine;
X⁹ is selected from the group aspartic acid, glutamic acid, valine, serine and threonine; and
X¹⁰ is selected from the group glycine, alanine, threonine, aspartic acid and glutamic acid.

In some embodiments, X¹ is valine. In some embodiments, X² is asparagine. In some embodiments, X³ is valine. In some embodiments, X⁴ is proline. In some embodiments, X⁵ is arginine. In some embodiments, X⁶ is alanine. In some embodiments, X⁷ is serine. In some embodiments, X⁸ is valine. In some embodiments, X⁹ is aspartic acid. In some embodiments, X¹⁹ is glycine.

In some embodiments, the MIF-mimic comprises any 5 or more consecutive peptide of Formula (II).

In some embodiments, the MIF-Mimic comprises 5 or more consecutive amino acids of human MIF₁₋₄₅ (numbering includes the first methionine). In some embodiments, the MIF-mimic comprises 5 or more consecutive amino acids of murine MIF₁₋₄₅. In some embodiments, the MIF-mimic comprises 5 or more consecutive amino acids of porcine MIF₁₋₄₅. In some embodiments, the MIF-mimic comprises 5 or more consecutive amino acids of bovine MIF₁₋₄₅. In some embodiments, the MIF-mimic comprises 5 or more consecutive amino acids of rat MIF₁₋₄₅.

In some embodiments, the MIF-mimic comprises one or more of the peptides selected from Table 2. In some embodiments, the MIF-mimic comprises N- and/or C-terminal chemical modifications to improve ADME-PK. In some embodiments, MIF-mimic comprises non-natural amino acids. In some embodiments, MIF-mimic comprises cyclical variants.

TABLE 2

| | |
|---|---|
| CTNVPRASVPDGC | NVPRASVPDG |
| CVPRASC | NVPRASVPD |
| VNTNVPRASVPDGFLSEL | NVPRASVP |
| NTNVPRASVPDGFLSEL | VPRASVP |
| TNVPRASVPDGFLSEL | PRASVP |
| NVPRASVPDGFLSEL | VPRASVPDGFL |
| VPRASVPDGFLSEL | VPRASVPDGF |
| PRASVPDGFLSEL | VPRASVPDG |
| RASVPDGFLSEL | VPRASVPD |
| ASVPDGFLSEL | VPRASVP |
| SVPDGFLSEL | VPRAS |
| VPDGFLSEL | MPMFIVNTNVPRASVPDGFLSEC |
| NVPRASVPDGFLSE | MPMFIVNTNVPRASV |
| NVPRASVPDGFLS | FIVNTNVPRASVPDG |
| NVPRASVPDGFL | NTNVPRASVPDGFLS |
| NVPRASVPDGF | VPRASVPDGFLSELT |

In some embodiments, the MIF-mimic adopts structural or functional features similar to the amino acid residues 65-94 (numbering includes the first methionine). In some embodiments, the MIF-mimic is a peptide. In some, embodiments, the MIF-mimic comprises a peptide of Formula (III):

I/L-G-X¹-X²-X³-X⁴-X⁵-X⁶-N-X⁷-X⁸-X⁹-X¹⁰-X¹¹-X¹²-L/I-X¹³-X¹⁴-X¹⁵-X¹⁶-X¹⁷X¹⁸-X¹⁹-L/V-X²⁰-I-X²¹-X²²-X²³-X²⁴

wherein:
X¹ is selected from the group consisting of lysine, arginine, cysteine, serine and alanine;
X² is selected from the group consisting of isoleucine, valine and phenylalanine;
X³ is selected from the group consisting of glycine, asparagine and serine;
X⁴ is selected from the group consisting of glycine, proline, alanine, aspartic acid and glutamic acid;
X⁵ is selected from the group consisting of alanine, proline, lysine, arginine, asparagine, aspartic acid and glutamic acid;
X⁶ is selected from the group consisting of glutamine, valine, lysine, arginine, leucine, aspartic acid and glutamic acid;
X⁷ is selected from the group consisting of lysine, arginine, asparagine, isoleucine and valine;
X⁸ is selected from the group consisting of serine, asparagine, glutamine, aspartic acid, glutamic acid, lysine and arginine;
X⁹ is selected from the group consisting of tyrosine, histidine and asparagine;
X¹⁰ is selected from the group consisting of serine, threonine and alanine;
X¹¹ is selected from the group consisting of lysine, aspartic acid, glutamic acid, alanine, serine and glycine;
X¹² is selected from the group consisting of leucine, glutamine, lysine, arginine, leucine, serine and alanine;
X¹³ is selected from the group consisting of cysteine, tyrosine, phenylalanine, serine, alanine and threonine;
X¹⁴ is selected from the group consisting of glycine, aspartic acid, glutamic acid, lysine and arginine;
X¹⁵ is selected from the group consisting of leucine, glutamine, isoleucine, histidine and phenylalanine;
X¹⁶ is selected from the group consisting of leucine, methionine, isoleucine and cysteine;
X¹⁷ is selected from the group consisting of alanine, threonine, serine, arginine, lysine, alanine, glutamine and glycine;
X¹⁸ is selected from the group consisting of glutamic acid, aspartic acid, lysine and arginine;
X¹⁹ is selected from the group consisting of arginine, histidine, glutamine, aspartic acid, glutamic acid, glycine, threonine and lysine;
X²⁰ is selected from the group consisting of arginine, histidine, glycine, asparagine, lysine, arginine, aspartic acid and glutamic acid;
X²¹ is selected from the group consisting of serine, aspartic acid, glutamic acid, lysine, arginine and proline;
X²² is selected from the group consisting of proline, alanine, lysine, arginine and glycine;

X²³ is selected from the group consisting of aspartic acid, glutamic acid, asparagine and alanine; and

X²⁴ is selected from the group consisting of histidine, tyrosine, lysine and arginine.

In some embodiments, X¹ is lysine. In some embodiments, X² is isoleucine. In some embodiments, X³ is glycine. In some embodiments, X⁴ is glycine. In some embodiments, X⁵ is alanine. In some embodiments, X⁶ is glutamine. In some embodiments, X⁷ is arginine. In some embodiments, X⁸ is serine. In some embodiments, X⁹ is tyrosine. In some embodiments, X¹⁰ is serine. In some embodiments, X¹¹ is lysine. In some embodiments, X¹² is leucine. In some embodiments, X¹³ is cysteine. In some embodiments, X¹⁴ is glycine. In some embodiments, X¹⁵ is leucine. In some embodiments, X¹⁶ is leucine. In some embodiments, X¹⁷ is alanine. In some embodiments, X¹⁸ is glutamic acid. In some embodiments, X¹⁹ is arginine. In some embodiments, X²⁰ is arginine. In some embodiments, X²¹ is serine. In some embodiments, X²² is proline. In some embodiments, X²³ is aspartic acid. In some embodiments, X²⁴ is arginine.

In some embodiments, the MIF-mimic comprises any 5 or more consecutive peptide of Formula (III).

In some embodiments, the MIF-mimic comprises 5 or more consecutive amino acids of human MIF₆₅₋₉₄. In some embodiments, the MIF-mimic comprises 5 or more consecutive amino acids of murine MIF₆₅₋₉₄. In some embodiments, the MIF-mimic comprises 5 or more consecutive amino acids of porcine MIF₆₅₋₉₄. In some embodiments, the MIF-mimic comprises 5 or more consecutive amino acids of bovine MIF₆₅₋₉₄. In some embodiments, the MIF-mimic comprises 5 or more consecutive amino acids of rat MIF₆₅₋₉₄.

In some embodiments, the MIF-mimic comprises one or more of the peptides selected from Table 3. In some embodiments, the MIF-mimic comprises N- and/or C-terminal chemical modifications to improve ADME-PK. In some embodiments, MIF-mimic comprises non-natural amino acids. In some embodiments, MIF-mimic comprises cyclical variants.

TABLE 3

| | |
|---|---|
| CSLHSIGKIGGAQNR | IAVHVVPDQLMAFGGSSEPCALCSLHSIGKIGGAQNRSYSKLL |
| IGKIGGAQNRSYSKL | IAVHVVPDQLMAFGGSSEPCALCSLHSIGKIGGAQNRSY |
| HSIGKIGGAQNRSYSKLLCGLL | IAVHVVPDQLMAFGGSSEPCALCSLHSIGKIGGAQ |
| HSIGKIGGAQNRSYSKLLCG | IAVHVVPDQLMAFGGSSEPCALCSLHSIGKI |
| HSIGKIGGAQNRSYSKLL | IAVHVVPDQLMAFGGSSEPCALCSLHS |
| HSIGKIGGAQNRSYSK | IAVHVVPDQLMAFGGSSEPCALC |
| HSIGKIGGAQNRSYS | IAVHVVPDQLMAFGGSSEP |
| IGKIGGAQNRSYSKLLC | IAVHVVPDQLMAFGG |
| KIGGAQNRSYSKLLC | IAVHVVPDQLM |
| GGAQNRSYSKLLCGLLAERLRI | IAVHVVPDQLMAFGGSSEPCALCSLHSIGKIGGAQNRSYSKLL |
| AQNRSYSKLLCGLLAERLRI | VVPDQLMAFGGSSEPCALCSLHSIGKIGGAQNRSYSKLL |
| NRSYSKLLCGLLAERLRI | QLMAFGGSSEPCALCSLHSIGKIGGAQNRSYSKLL |
| SYSKLLCGLLAERLRI | FGGSSEPCALCSLHSIGKIGGAQNRSYSKLL |
| YSKLLCGLLAERLRI | SEPCALCSLHSIGKIGGAQNRSYSKLL |
| GAQNRSYSKLLGGLLAE | ALCSLHSIGKIGGAQNRSYSKLL |
| GAQNRSYSKLLCGLL | LHSIGKIGGAQNRSYSKLL |
| QNRSYSKLLCGLLAE | GKIGGAQNRSYSKLL |
| HSIGKIGGAQNRSY | IGGAQNRSYSKLL |
| HSIGKIGGAQNR | QNRSYSKLL |
| HSIGKIGGAQNRSYSK | IGKIGGAQNRSYSKL |
| IGKIGGAQNRSYSKLLC | IGKIGGAQ |
| KIGGAQNRSYSKLLC | linear (CIGKIGGAQC) |
| KIGGAQNRSYS | cyclo (CIGKIGGAQC) |
| GAQNRSYSKLLCGLLAE | RSYSKLLCGLLAE |
| GAQNRSYSKLLCGLL | linear (CRSYSKLLCGLLAEC) |
| GAQNRSYSKLLCG | cyclo (CRSYSKLLCGLLAEC) |
| GAQNRSYSKLL | CGLLAERLRISPDR |
| QNRSYSKLLCGLLAE | linear (CGLLAERLRISPDRC) |
| RSYSKLLCGLLAE | Cyclo (CGLLAERLRISPDRC |
| YSKLLCGLLAE | |

### E. CD74 Mimics

In some embodiments, the modulator of MIF is an agent that disrupts the ability of CD74 to forth a signaling complex with CXCR2, CXCR4, MIF, or a combination thereof. In some embodiments, the modulator of MIF is a CD74-like peptide that mimics part or all of a CD74 domain (e.g., the C-terminal/extracellular (lumenal) domain). In some embodiments, the CD74-mimic binds to MIF, CXCR2, and/or CXCR4 and thus prevents CD74 from binding to MIF, CXCR2, and/or CXCR4.

In some embodiments, the CD74-mimic adopts structural or functional features similar to CD74. In some embodiments, the CD74-mimic is a peptide.

In some embodiments, the CD74-mimic comprises 5 or more consecutive amino acids of human CD74. In some embodiments, the CD74-mimic comprises 5 or more consecutive amino acids of bovine CD74. In some embodiments, the CD74-mimic comprises 50r more consecutive amino acids of porcine CD74. In some embodiments, the CD74-mimic comprises 5 or more, consecutive amino acids of murine CD74. In some embodiments, the CD74-mimic comprises 5 or more consecutive amino acids of rat. CD74.

In some embodiments, the CD74-mimic comprises one or more of the peptides selected from Table 3. In some embodiments, the CD-74-mimic comprises N- and/or C-terminal chemical modifications to improve ADME-PK. In some embodiments, CD74-mimic comprises non-natural amino acids. In some embodiments, CD74-mimic comprises cyclical variants.

TABLE 4

| | |
|---|---|
| AYFLYQQQ | TKYGNMTEDHVMHLL |
| QQQGRLDKLTVTGRL | HVMHLLQNADPLKVY |
| GRLDKLTVTSQNLQL | DPLKVYPPLKGSFPE |
| SQNLQLENLRM | KGSFPENLRHLKNTM |
| TVTGRLDKLTVTSQN | HLKNTMETIDWKVFE |
| TVTSQNLQLENLRM | DWKVFESWMHHWLLF |
| LENLRMKLPKPPKPV | HHWLLFEMSRHSLEQ |
| KLPKPPKPVSKMRMA | RHSLEQKPTDAPPKE |
| SKMRMATPL | DAPPKESLELEDPSS |
| LMQALPMGALPQGPM | LEDPSSGLGVTKQDL |
| LPQGPMQNATKYGNM | VTKQDLGPVPM |

### E. CXCR2/CXCR4Mimics

In some embodiments, the modulator of MIF is an agent that disrupts the ability of CXCR2 and/or CXCR4 to form a signaling complex with CD74 and/or MIF.

In some embodiments, the modulator of MIF is a CXCR2-like peptide that mimics part or all of a CXCR2 domain. In some embodiments, the modulator of MIF is a CXCR2-like peptide that mimics part or all of the CXCR2 extracellular loop 1 and/or extracellular loop 2. In some embodiments, the CXCR2-mimic binds to MIF and/or CD74 and thus, prevents CXCR2 froth binding to MIF and/or CD74.

In some embodiments, the modulator of MIF is a CXCR4-like peptide that mimics part or all of a CXCR4 domain. In some embodiments, the modulator of MIF is a CXCR4-like peptide that mimics part or all of the CXCR4 extracellular loop 1 and/or extracellular loop 2. In some embodiments, the modulator of MIF is a CXCR4-like peptide that mimics part or all of the CXCR4 amino acids 182-202 (SEADDRYICDRFYPNDLWVVV). In some embodiments the modulator of MIF is a CXCR4-like peptide that mimics part or all of the CXCR4 amino acids 185-199 (DDRYICDRFYPNDLW). In some embodiments, the CXCR4-mimic binds to MIF and/or CD74 and thus prevents CXCR4 from binding to MIF and/or CD74.

In some embodiments, the CXCR4-mimic or the CXCR2 mimic comprises one or more of the peptides selected from Table 4. In some embodiments, the mimic comprises N- and/or C-terminal chemical, modifications to improve ADME-PK. In some embodiments, the mimic comprises non-natural amino acids. In some embodiments, mimic comprises cyclical variants.

TABLE 5

| | |
|---|---|
| DLSNYSYSSTLPPFL | MRTQVIQ |
| DLSNYSYSSTLPP | MRTQV |
| DLSNYSYSSTL | CERRNHIDRALDA |
| DLSNYSYSS | CERRNHIDRAL |
| DLSNYSY | CERRNHIDR |
| DLSNY | CERRNHI |
| KVNGWIFGTFL | CERRN |
| KVNGWIFGT | DRYICDRFYPNDL |
| KVNGWIF | DRYICDRFYPN |
| KVNGW | DRYICDRFY |
| RRTVYSSNVSPAC | DRYICDR |
| RRTVYSSNVSP | DRYIC |
| RRTVYSSNV | ICDRFYPNDLWVV |
| RRTVYSS | ICDRFYP |
| RRTVY | ICDRF |
| EDMGNNTANWRML | RFYPNDLWVVVFQ |
| EDMGNNTANWR | RFYPNDLWVVV |
| EDMGNNTAN | RFYPNDLWV |
| EDMGNNT | RFYPNDL |
| EDMGN | RFYPN |
| MRTQVIQETCERR | |
| MRTQVIQETCE | |
| MRTQVIQET | |

### F. Fusion Peptide

In some embodiments, the modulator of MIF is an agent that disrupts the ability of MIF to form a signaling complex with CXCR2, CXCR4, CD74, or a combination thereof. In some embodiments, the modulator of MIF is a fusion peptide that binds both the N-loop domain of MIF and the pseudo-ELR domain of MIF.

In some embodiments, the peptides that comprise the fusion peptide are derived from human MIF, bovine MIF, porcine MIF, murine MIF, rat MIF, or a combination thereof. In some embodiments, the peptides that comprise the fusion peptide are artificially constructed.

In some embodiments, the fusion peptide comprises at least one peptide that adopts structural or functional features similar to the N-loop motif of MIF, and at least one peptide that adopts structural or functional features similar to the pseudo-ELR loop of MIF. In some embodiments, the fusion peptide comprises (a) a first peptide that adopts structural or functional features similar to the N-loop motif of MIF; and (b) a second peptide that adopts structural or functional features similar to the pseudo-ELR loop of MIF. In some embodiments, the fusion peptide comprise (a) a first peptide that adopts structural or functional features similar to the N-loop motif of MIF; (b) a second peptide that adopts structural or functional features similar to a first portion of the pseudo-ELR loop of MIF; and (c) a third peptide that adopts structural or functional features similar to a second portion of the pseudo-ELR loop of MIF.

In some embodiments, the fusion peptide comprise (a) a first peptide that adopts structural or functional features similar to the N-loop motif of MIF; and (b) a second peptide that adopts structural or functional features similar to the pseudo-ELR loop Of MIF; wherein the first peptide and the second peptide are chemically linked. In some embodiments, the fusion peptide comprise (a) a first peptide that adopts structural or functional features similar to the N-loop motif of MIF; (b) a second peptide that adopts structural or functioning features similar to a first portion of the pseudo-ELR loop of MIF; and (c) a third peptide that adopts structural or functional features similar to a second portion of the pseudo-ELR loop of MIF; wherein the first peptide, the second peptide, and the third peptide are chemically linked.

In some embodiments, the fusion peptide comprises (a) a first peptide having the sequence MAFGGSSEPC; and (b) a second peptide having the sequence NVPRA. In some embodiments, the fusion peptide comprises (a) a first peptide having the sequence MAFGGSSEPC; (b) a second peptide having the sequence NVPRA; and (c) a third peptide having the sequence SVPDG.

In some embodiments, the methods and compositions disclosed herein comprise (a) a first peptide having the sequence LQDP; and (b) a second peptide having the sequence NVPRA.

In some embodiments, the first peptide and the second peptide are directly bound to each other (e.g., via a covalent or ionic bond).

### Linkers

In some embodiments, at least one peptide that adopts structural or functional features similar to the N-loop motif of MIF and at least one peptide that adopts structural or functional features similar to the pseudo-ELR loop of MIF are indirectly bound to each other (e.g., via a linker). In some embodiments, at least one peptide that adopts structural or functional features similar to the N-loop motif of MIF and at least one peptide that adopts structural or functional features similar to the pseudo-ELR loop of MIF are bound by a linker.

In some embodiments, the linker binds (a) a first peptide that adopts structural or functional features similar to the N-loop motif Of MIF; and (b) a second peptide that adopts structural or functional features similar to the pseudo-ELR loop of MIF. In some embodiments, the fusion peptide is a peptide of Formula (IV):

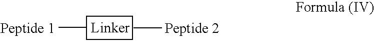

wherein Peptide 1, and Peptide 2 are selected from any peptide disclosed herein.

In some embodiments, the linker binds (a) a first peptide that adopts structural or functional features similar to the N-loop motif of MIF; (b) a second peptide that adopts structural or functional features similar to a first portion of the pseudo-ELR loop of MIF; and (c) a third peptide that adopts structural or functional features similar to, a second portion of the pseudo-ELR loop of MIF. In some embodiments, the fusion peptide is a peptide of Formula (Y):

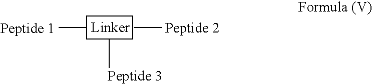

wherein Peptide 1, Peptide 2, and Peptide 3 are selected from any peptide disclosed herein.

As used herein, a “linker” is any molecule capable of binding (e.g., covalently) to multiple peptides. In some embodiments, the linker binds to the peptide by a covalent linkage. In some embodiments, the covalent linkage comprises a ether bond, thioether bond, amine bond, amide bond, carbon-carbon bond, carbon-nitrogen bond, carbon-oxygen bond, or carbon-sulfur bond.

In some embodiments, the linker is flexible. In some embodiments, the linker is rigid. In some embodiments, the linker is long enough to allow the fusion peptide to bind to both the pseudo-ELR and N-loop domains of MIF.

In some embodiments, the linker binds to two peptides. In some embodiments, the linker binds to three peptides.

In some embodiments, a linker described herein binds to the C-terminus of one or more of the peptides that form the fusion peptide. In some embodiments, the linker binds to the N-terminus of one or more of the peptides that form the fusion peptide. In some embodiments, a linker described herein binds to the C-terminus of one or more of the peptides and the N-terminus of any remaining peptides.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker is an alkyl. In some embodiments, the linker is heteroalkyl.

In some embodiments, the linker is an alkylene. In some embodiments, the linker is an alkenylene. In some embodiments, the linker is an alkynylene. In some embodiments, the linker is a heteroalkylene.

An “alkyl” group refers to an aliphatic hydrocarbon group. The alkyl moiety may be a saturated alkyl or an unsaturated alkyl. Depending on the structure, an alkyl group can be a monoradical or a diradical (i.e., an alkylene group).

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 6 carbon atoms. The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

In some embodiments, the linker comprises a ring structure (e.g., an aryl). As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyl groups include groups having from 3 to 10 ring atoms. Cycloalkyls include, but are not limited to, cyclopropyl, cyclobutyl, cyclopentyl, cyclohexyl, cycloheptyl, and cyclooctyl.

In some embodiments, the ring is a cycloalkane. In some embodiments, the ring is a cycloalkene.

In some embodiments, the ring is an aromatic ring. The term “aromatic” refers to a planar ring having a delocalized π-electron system containing 4n+2π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

In some embodiments, the ring is a heterocycle. The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, okepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, may be C-attached or N-attached where such is possible. For instance, a group derived from pyrrole may be pyrrol-1-yl (NV attached)or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

In some embodiments, the ring is fused. The term “fused” refers to structures in which two or more rings share one or more bonds. In some embodiments, the ring is a dimer. In some embodiments, the ring is a trimer. In some embodiments, the ring is a substituted.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e., a heteroatom). Heterocycle includes heteroaryl and heterocycloalkyl. Carbocycles and heterocycles can be optionally substituted.

In some embodiments, the linker is substituted. The term “optionally substituted” or “substituted” means that the referenced group may be substituted with one or more additional group(s) individually and independently selected from C₁-C₆alkyl, C₃-C₈cycloalkyl, aryl, heteroaryl, C₂-C₆heteroalicyclic, hydroxy, C₁-C₆alkoxy, aryloxy, C₁-C₆alkylthio, arylthio, C₁-C₆alkylsulfoxide, arylsulfoxide, C₁-C₆alkylsulfone, arylsulfone, cyano, halo, C₂-C₈acyl, C₂-C₈acyloxy, nitro, C₁-C₆haloalkyl, C₁-C₆-fluoroalkyl, and amino, including C₁-C₆alkylamino, and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(═O)—, —C(═O)NH—, S(═O)₂NH—, —NHS(═O)₂—, —OC(═O)NH—, —NHC(═O)—, —(C₁-C₆alkyl)-, or —(C₂-C₆alkenyl)-; and each R^{s} is independently selected from H, (C₁-C₄alkyl), (C₃-C₈cycloalkyl), heteroaryl, aryl, and C_{I}-C₆heteroalkyl. Optionally substituted non-aromatic groups may be substituted with one or more oxo (═O). The protecting groups that may form the protective derivatives of the above substituents are known to those of skill in the art.

In some embodiments, the linker is an amino acid. In some embodiments, the fusion peptide is a peptide of Formula (VI):

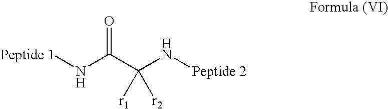

wherein Peptide 1, and Peptide 2 are selected from any peptide disclosed herein.

In some embodiments, the linker is an artificial amino acid. In some embodiments, the linker is a β-amino acid. In some embodiments, the linker is a γ-amino acid.

In some embodiments, the linker is a polyethylene glycol (PEG). In some embodiments, the linker is a diamino acid. In some embodiments, the linker is diaminopropionic acid.

In some embodiments, the linker is hydrolyzible.

By way of non-limiting example, the fusion peptide is:

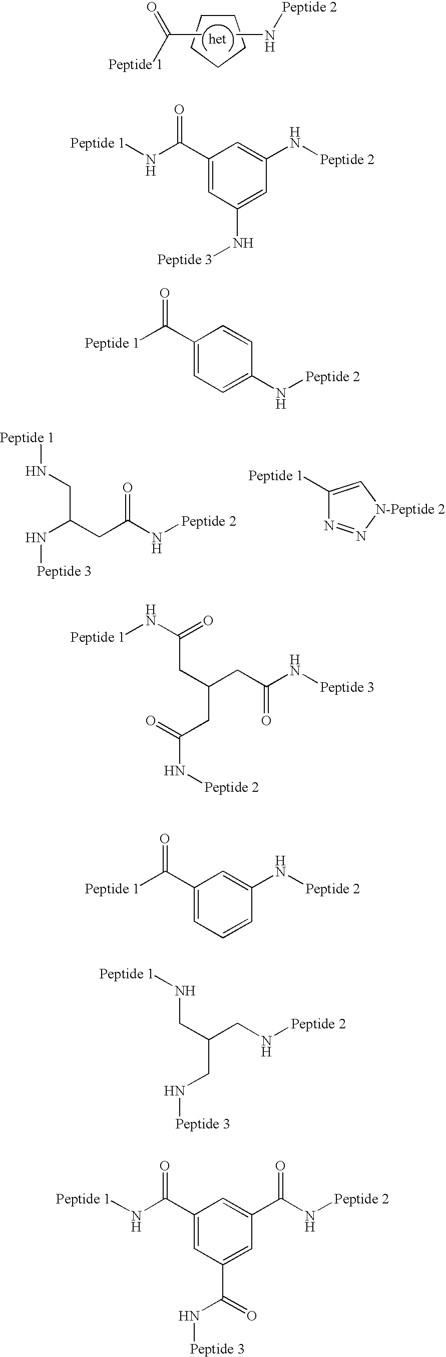

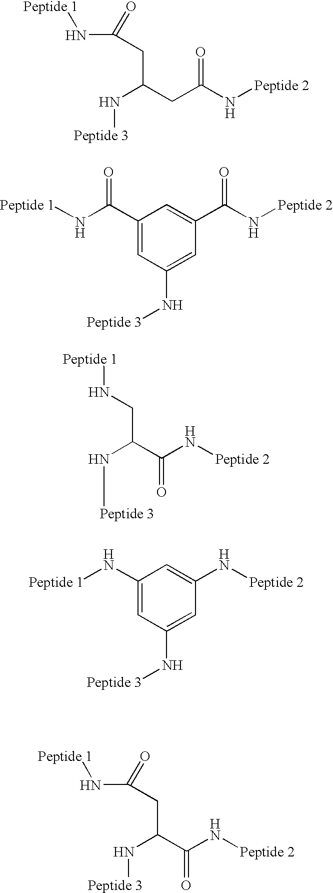

wherein Peptide 1, Peptide 2, and Peptide 3 are selected from any peptide disclosed herein.

### E. MIF Trimerization Modulating Agents

In some embodiments, the modulator of MIF is an agent that modulates the ability of MIF to form a homo-multimer. In some embodiments, the modulator of MIF is an agent that disrupts the ability of MIF to form a trimer. In some embodiments, an inflammatory disease, disorder, condition, or symptom is treated by promoting MIF trimerization.

In certain instances, functionally-active MIF comprises three MIF peptide sequences (i.e., a trimer). In certain instances, the pseudo-ELR loops of each MIF polypeptide form a ring in the trimer. In certain instances, the N-loop motifs of each MIF polypeptide extend Outwards from the pseudo-ELR ring (see FIG. 10). In certain instances, disruption of the timer disrupts the high affinity binding of MIF to its target receptors.

In certain instances, residues 38-44 of one subunit interact with residues 48-50 of a second subunit. In certain instances; residues 96-102 of one subunit interact with residues 107-109 of a second subunit. In certain instances, a domain on one subunit formed by N73 R74 S77 K78 C81 (numbering includes the first methionine) interacts with N110 S111 T112 (numbering includes the first methionine) of a second subunit.

In some embodiments, a MIF trimerization disrupting agent is derived from and/or incorporates any or all of amino acid residues 38-44 of MIF (e.g., human, bovine, procine, murine, or rat). In some embodiments, a MIF trimerization disrupting agent is a peptide derived from and/or incorporates any or all of amino acid residues 48-50 of MIF (e.g., human, bovine, procine, murine, or rat). In some embodiments, a MIF trimerization disrupting agent is a peptide derived from and/or incorporates any or all of amino acid residues 57-66 of MIF (e.g., human, bovine, procine, murine, or rat). In some embodiments, a MIF trimerization disrupting agent is a peptide derived from and/or incorporates any or all of amino acid residues 61-70 of MIF (e.g., human, bovine, procine, murine, or rat). In some embodiments, a MIF trimerization disrupting agent is a peptide derived from and/or incorporates any or all of amino acid residues 96-102 of MIF (e.g., human, bovine, procine, murine, or rat). In some embodiments, a MIF trimerization disrupting agent is a peptide derived from and/or incorporates any or all of amino acid residues 107-109 of MIF (e.g., human, bovine, procine, murine, or rat). In some embodiments, a MIF trimerization disrupting agent is a peptide derived from and/or incorporates any or all of amino acid residues N73, R74, S77, K78, and C81 of MIF (e.g., human, bovine, procine, murine, or rat) (numbering includes the first methionine). In some embodiments, a MIF trimerization disrupting agent is a peptide derived from and/or incorporates any or all of amino acid residues N 110, S111, and T112 of MIF (e.g., human, bovine, procine, murine, or rat) (numbering includes the first methionine).

In some embodiments, a MIF trimerization disrupting agent is a peptide derived from and/or incorporates any or all of amino acid residues 57-66 of MIF (numbering includes the first methionine). In some embodiments, a MIF trimerization disrupting agent is a peptide of Formula (VII):

X¹-X²-X³-X⁴-X⁵-X⁶-X⁷-S/A-I-G

wherein:
X¹ is selected from the group consisting of cysteine, alanine, serine, and threonine;
X² is selected from the group consisting of alanine, proline, glycine and cysteine;
X³ is selected from the group consisting of leucine, valine and pheynylalanine;
X⁴ is selected from the group consisting of cysteine, glycine, threonine and isoleucine;
X⁵ is selected from the group consisting of serine, valine, glutamine and asparagine;
X⁶ is selected from the group consisting of leucine, valine, isoleucine and methionine; and
X⁷ is selected from the group consisting of histidine, cysteine, lysine, arginine, and leucine.

In some embodiments, X¹ is. In some embodiments, X² is. In some embodiments, X³ is. In some embodiments, X⁴ is. In some embodiments, X⁵ is. In some embodiments, X⁶ is. In some embodiments, X⁷ is.

In some embodiments, the MIF-mimic comprises any 5 or more consecutive peptide of Formula (VIII).

In some embodiments, the MIF-mimic comprises 5 or more consecutive amino acids of human MIF₅₇₋₆₆. In some embodiments, the MIF-mimic comprises 5 or more consecutive amino acids of murine MIF₅₇₋₆₆. In some embodiments, the MIF-mimic comprises 5 or more consecutive amino acids of porcine MIF₅₇₋₆₆. In some embodiments, the MIF-mimic comprises 5 or more consecutive amino acids of bovine MIF₅₇₋₆₆. In some embodiments, the MIF-mimic comprises 5 or more consecutive amino acids of rat MIF₅₇₋₆₆.

In some embodiments, a MIF trimerization disrupting agent is an antibody that binds to any or all of amino acid residues 38-44 of MIF. In some-embodiments, a MIF trimerization disrupting agent is an antibody that binds to any or all of amino acid residues 48-50 of MIF. In some embodiments, a MIF trimerization disrupting agent is an antibody that binds to any or all of amino acid residues 57-66 of MIF. In some embodiments, a MIF trimerization disrupting agent is an antibody that binds to any or all of amino acid residues 61-70 of MIF. In some embodiments, a MIF trimerization disrupting agent is an antibody that binds to any or all of amino acid residues 96-102 of MIF. In some embodiments, a MIF trimerization disrupting agent is an antibody that binds to any or all of amino acid residues 107-109 of MIF. In some embodiments, a MIF trimerization disrupting agent is an antibody that binds to any or all of amino acid residues N73, R74, S77, K78, and C81 of MIF. In some embodiments, a MIF trimerization disrupting agent is an antibody that binds to any or all of amino acid residues N110, S111, and T112 of MIF.

In some embodiments, a MIF trimerization disrupting agent is a small molecule that binds to any or all of amino acid residues 38-44 of MIF. In some embodiments, a MIF trimerization disrupting agent is a small molecule that binds to any or all of amino acid residues 48-50 of MIF. In some embodiments, a MIF trimerization disrupting agent is a small molecule that binds to any or all of amino acid residues 57-66 of MIF. In some embodiments, a MIF trimerization disrupting agent is a small molecule that binds to any or all of amino acid residues 61-70 of MIF. In some embodiments, a MIF trimerization disrupting agent is a small molecule that binds to any or all of amino acid residues 96-102 of MIF. In some embodiments, a MIF trimerization disrupting agent is a small molecule that binds to any or all of amino acid residues 107-109 of MIF. In some embodiments, a MIF trimerization disrupting agent is a small molecule that binds to any or all of amino acid residues N73, R74, S77, K78, and C81 of MIF. In some embodiments, a MIF trimerization disrupting agent is a small molecule that binds to any or all of amino acid residues N110, S111, and T112 of MIF.

In some embodiments, a MIF trimerization disrupting agent is a peptibody that binds to any or all of amino acid residues 38-44 of MIF. In some embodiments, a MIF trimerization disrupting agent is a peptibody that binds to any or all of amino acid residues 48-50 of MIF. In some embodiments, a MIF trimerization disrupting agent is a peptibody that binds to any or all of amino acid residues 57-66 of MIF. In some embodiments, a MIF trimerization disrupting agent is a peptibody that binds to any or all of amino acid residues 61-70 of MIF. In some embodiments, a MIF trimerization disrupting agent is a peptibody that binds to any or all of amino acid residues 96-102 of MIF. In some embodiments, a MIF trimerization disrupting agent is a peptibody that binds to any or all of amino acid residues 107-109 of MIF. In some embodiments, a MIF trimerization disrupting agent is a peptibody that binds to any or all of amino acid residues N73, R74, S77, K78, and C81 of MIF. In some embodiments, a MIF trimerization disrupting agent is a peptibody that binds to any or all of amino acid residues N110, S111, and T112 of MIF.

### F. Peptide Mimetics

In some embodiments, a peptide mimetic is used in place of the peptides described herein, including for use in the treatment or prevention of an inflammatory disorder.

Peptide mimetics and peptide-based inhibitors) are developed using, for example, computerized molecular Modeling. Peptide mimetics are designed to include structures having one or more peptide linkages optionally replaced by a linkage selected from the group consisting of: —CH₂NH—, —CH₂S—, —CH₂—CH₂—, —CH═CH-(cis and trans), —CH═CF-(trans), —CoCH₂—, —CH(OH)CH₂—, and —CH₂SO—, by methods well known in the art. In some embodiments such peptide mimetics have greater chemical stability, enhanced pharmacological properties (half-life, absorption, potency, efficacy, etc.), altered specificity (e.g., a broad-spectrum of biological activities), reduced antigenicity, and are more economically prepared. In some embodiments peptide mimetics include covalent attachment of one or more labels or conjugates, directly or through a spacer (e.g., an amide group), to non-interfering positions(s) on the analog that are predicted by quantitative structure-activity data and/or molecular modeling. Such non-interfering positions generally are positions that do not form direct contacts with the receptor(s) to which the peptide mimetic specifically binds to produce the therapeutic effect. In some embodiments, systematic substitution of one or more amino acids of a consensus sequence with a D-amino acid of the same type (e.g., D-lysine in place of L-lysine) are used to generate more stable peptides with desired properties.

Phage display peptide libraries have emerged as a technique in generating peptide mimetics (Scott, J. K. et al. (1990) Science 249:386; Devlin, J. J. et al. (1990) Science 249:404;. U.S. Pat. No. 5,223,409, U.S. Pat. No. 5,733,731; U.S. Pat. No. 5,498,530; U.S. Pat. No. 5,432,018;U.S. Pat. No. 5,338,665;U.S. Pat. No. 5,922,545; WO 96/40987 and WO 98/15833 (each of which is incorporated by reference for such disclosure). In such libraries, random peptide sequences are displayed by fusion with coat proteins of filamentous phage. Typically, the displayed peptides are affinity-eluted against an antibody-immobilized extracellular domain (in this case PF4 or RANTES. In some embodiments peptide mimetics are isolated by biopanning (Nowakowski, G. S, et al. (2004) Stem Cells 22:1030-1038). In some embodiments whole cells expressing MIF are used to screen the library utilizing PACs to isolate phage specifically bound cells. The retained phages are enriched by successive rounds of biopanning and repropagation. The best binding peptides are sequenced to identify key residues within one or more structurally related families of peptides. The peptide sequences also suggest which residues to replace by alanine scanning or by mutagenesis at the DNA level. In some embodiments mutagenesis libraries are created and screened to further optimize the sequence of the best binders. Lowman (1997) Ann. Rev. Biophys. Biomol. Struct. 26:401-24.

In some embodiments structural analysis of protein-protein interaction is used to suggest peptides that mimic the binding activity of the polypeptides described herein. In some embodiments the crystal structure resulting from such an analysis suggests the identity and relative orientation of critical residues of the polypeptide, from which a peptide is designed. See, e.g., Takasaki, et al. (1997) Nature Biotech, 15: 1266-70.

In some, embodiments, the agent is a peptide or polypeptide. In some embodiments, the peptide is a peptide that mimics a peptide sequence as follows: VNTNVPPRASVPDGFLSELTQQLAQATGKPPQYIAVHVVPDQL and the corresponding feature/domain of at least one of a MIF monomer or MIF trimer; a peptide that mimics a peptide sequence as follows: PDQLMAFGGSSEPCALCSL and the corresponding feature/domain of at least one of a MIF monomer or MIF trimer; a peptide that mimics a peptide sequence as follows: VNTNVPPRASVPDGFLSELTQQLAQATGKPPQYIAVHVVPDQLMAFGGSSEPCALCSL and the corresponding feature/domain of at least one of a MIF monomer or MIF trimer; a peptide that mimics a peptide sequence as follows: PDQLIVIAFGGSSEPCALCSIIISI and the corresponding feature/domain of at least one of a MIF monomer or MIF trimer; or combinations thereof.

### IV. Combinations

Disclosed herein, in certain embodiments, are methods and pharmaceutical compositions for modulating a disorder of a cardiovascular system synergistic combination of (a) a therapeutically-effective amount of a modulator of MIF; and (b) a second active agent selected from an agent that treats a cardiovascular disorder (the “cardiovascular disorder agent”).

In some embodiments, combining a (a) cardiovascular disorder agent with (b) a therapeutically-effective amount of a modulator of MIF is synergistic and results in a more efficacious therapy. In some embodiments, the therapy is more efficacious as it treats cardiovascular disorders by multiple pathways. In some embodiments, the therapy is more efficacious as it treats cardiovascular disorders by multiple pathways and treats and/or ameliorates undesired inflammation resulting from the cardiovascular disorder agent.

In some embodiments, the co-administration of (a) a therapeutically-effective amount of a modulator of MIF; and (b) a second active agent selected from an agent that treats a cardiovascular disorder rescues a mammal from inflammation partially or fully caused by the cardiovascular disorder agent. In certain instances, statins (e.g., atorvastatin, lovastatin and simvastatin) can induce inflammation. In certain instances, administration of a statin results (partially or fully) in myositis.

In some embodiments, the co-administration of (a) a therapeutically-effective amount of a modulator of MIF; and (b) a cardiovascular disorder agent allows (partially or fully) a medical professional to increase the prescribed dosage of the cardiovascular disorder agent. In certain instances, statin-induced myositis is dose-dependent. In some embodiments, prescribing the modulator of MIF allows (partially or fully) a medical professional to increase the prescribed dosage of statin.

In some embodiments, the co-administration of (a) a therapeutically-effective amount of a modulator of MIF; and (b) a cardiovascular disorder agent enables (partially or fully) a medical professional to prescribe the cardiovascular disorder agent (i.e., co-administration rescues the cardiovascular disorder agent).

HDL-raising therapies include, but are not limited to, niacin, fibrates, statins, Apo-A 1 mimetic peptides (e.g., DF-4, Novartis), apoA-I transcriptional up-regulators (e.g., RVX-208, Resverlogix), ACAT inhibitors (e.g., avasimibe; IC-976, Pfizer, MCC-147, Mitsubishi Pharma), CETP modulators, or combinations thereof.

In some embodiments, the cardiovascular disorder agent raises HDL non-selectively. In some embodiments, the cardiovascular disorder agent down-regulates transcription of a CETP gene. In some embodiments, the second active agent is niacin.

In some embodiments, the modulator of MIF inhibits inflammation and treats a cardiovascular disorder by a modulating. MIF. In some embodiments, the cardiovascular disorder agent is a statin. In some embodiments, the cardiovascular disorder agent is atorvastatin; cerivastatin; fluvastatin; lovastatin; mevastatin; pitavastatin; pravastatin; rosuvastatin; simvastatin; simvastatin and ezetimibe; lovastatin and niacin, extended-release; atorvastatin and amlodipine besylate; simvastatin and niacin, extended-release; or combinations thereof. In some embodiments, the modulator of MIF and the statin synergistically treat a CVD by (1) decreasing the chemotaxis of leukocytes, (2) decreasing the synthesis of cholesterol, and (3) decreasing any undesired inflammation resulting from administration of the statin.

In some embodiments, the modulator of MIF inhibits inflammation and treats a cardiovascular disorder by modulating MIF. In some embodiments, the cardiovascular disorder agent reduces the risk of developing a cardiovascular disorder in individuals with low HDL with metabolic syndrome. In some embodiments, the cardiovascular disorder agent is a fibrate. In some embodiments, the cardiovascular disorder agent is bezafibrate; ciprofibiate; clofibrate; gemfibrozil; fenofibrate; or combinations thereof. In some: embodiments, the modulator of MIF and the fibrate synergistically treat a CVD by (1) decreasing the chethotaxis of leukocytes, and (2) increasing the concentration of HDL. In some embodiments, the modulator of MIF also decreases any undesired inflammation resulting from administration of the fibrate.

In some embodiments, the modulator of MIF inhibits inflammation and treats a cardiovascular disorder by modulating MIF. In some embodiments, the cardiovascular disorder agent selectively increases the levels of ApoA-I protein (e.g. by transcriptional induction of the gene encoding ApoA-I) and increases the production of nascent HDL (ApoAI-enriched). In some embodiments, the cardiovascular disorder agent is DF4 (Ac-D-W-F-K-A-F-Y-D-K-V-A-E-K-F-K-E-A-F-NH2); DF5; RVX-208 (Resverlogix); or combinations thereof. In some embodiments, the modulator of MIF and the ApoA1 modulator synergistically treat a CVD by (1) decreasing the chemotaxis of leukocytes, and (2) increasing the concentration of HDL. In some embodiments, the modulator of MIF also decreases any undesired inflammation resulting from administration of the ApoA1 modulator.

In some embodiments, the modulator of MIF inhibits inflammation and treats a cardiovascular disorder by modulating MIF. In some embodiments, the Cardiovascular disorder agent is an ACAT inhibitor. In some embodiments, the cardiovascular disorder agent is avasimibe; pactimibe sulfate (CS-505); CI-1011 (2,6-diisopropylphenyl [(2,4,6-triisopropylphenyl)acetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N[[-(2,2-dimethylpropyl)phenyl]methyl]-N-(hepthyl)urea), F-1394 ((1s,2s)-2-[3-(2,2-dimethylpropyl)-3-nonylureido]aminocyclohexane-1-yl 3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; or combinations thereof. In some embodiments, the modulator of MIF and the ACAT modulator synergistically treat a CVD by (1) decreasing the chemotaxis of leukocytes, and (2) decreasing (a) the production and release of apoB-containing lipoproteins and (b) foam cell formation. In some embodiments, the modulator of MIF also decreases any undesired inflammation resulting from administration of the ACAT inhibitor.

In some embodiments, the modulator of MIF inhibits inflammation and treats a cardiovascular disorder by modulating MIF. In some embodiments, the cardiovascular disorder agent (partially or completely) the inhibits activity of Cholesteryl Ester Transfer Protein (CETP). In some embodiments, the cardiovascular disorder agent is torcetrapib; anacetrapid; ITT-705 (Japan Tobacco/Roche); or combinations thereof. In some embodiments, the modulator of MIF and the CETP modulator synergistically treat a CVD by (1) decreasing the chemotaxis of leukocytes, and (2) decreasing the transfer cholesterol from HDL cholesterol to LDL. In some embodiments, the modulator of MIF also decreases any undesired inflammation resulting from administration of the CETP inhibitor.

Therapeutics used to treat acute corollary syndrome (ACS) and acute myocardial infarction (AMI) include, but are not limited to, Glycoprotein (GP) IIb/IIIa receptor antagonists, P2Y12 receptor antagonists, and Lp-PLA2-inhibitors.

In some embodiments, the modulator of MIF inhibits inflammation and treats a cardiovascular disorder by modulating MIF. In some embodiments, the cardiovascular disorder agent is a Glycoprotein (GP) IIb/IIIa receptor antagonist. In some embodiments, the cardiovascular disorder agent is abciximab; eptifibatide; tirofiban; roxifiban; variabilin; XV 459 (N(3)-(2-(3-(4-formamidinophenyl)isoxazolin-5-yl)acetyl)-N(2)-(1-butyloxycarbonyl)-2,3-diaminopropionate); SR 121566A (3-[N-{4-[4-(aminoiminomethyl)phenyl]-1,3-thiazol-2-yl}-N-(1-carboxymethylpiperid-4-yl)aminol propionic acid, trihydrochloride); FK419 ((S)-2-acetylamino-3-[(R)-[1-[3-(piperidin-4-yl) propionyl]piperidin-3-ylcarbonyl]amino]propionioacid trihydrate); or combinations thereof. In some embodiments, the modulator of MIF and the GP IIb/IIIa receptor antagonist synergistically treat a CVD by (1) decreasing the chemotaxis of leukocytes, and (2) inhibiting platelet aggregation. In some embodiments, the modulator of MIF also decreases any undesired inflammation resulting from administration of the GP IIb/IIIa receptor antagonist.

In some embodiments, the modulator of MIF inhibits inflammation and treats a cardiovascular disorder by modulating MIF. In some embodiments, the cardiovascular disorder agent is a P2Y12 receptor antagonist. In some embodiments, the cardiovascular disorder agent is clopidogiel; prasugrel; cangrelor; AZD6140 (AstraZeneca); MRS 2395 (2,2-Dimethyl-propionic acid 3-(2-chloro-6-methylaminopurin-9-yl)-2-(2,2-dimethyl-propionyloxymethyl)-propyl ester); BX 667 (Berlex Biosciences); BX 048 (Berlex Biosciences) or combinations thereof. In some embodiments, the modulator of MIF and the P2Y12 receptor antagonist synergistically treat a CVD by (1) decreasing the chemotaxis of leukocytes, and (2) inhibiting platelet aggregation. In some embodiments, the modulator of MIF also decreases any undesired inflammation resulting from administration of the P2Y12 receptor antagonist.

In some embodiments, the modulator of MIF inhibits inflammation and treats a cardiovascular disorder by modulating MIF. In some embodiments, the cardiovascular disorder agent is an Lp-PLA2 antagonist. In some embodiments, the second active agent is daratiladib (SB 480848); SB-435-495 (GlaxoSmithKline); SB-222657 (GlaxoSmithKline); SB-253514 (GlaxoSmithKline); or combinations thereof. In some embodiments, the modulator of MIF and Lp-PLA2 antagonist synergistically treat a CVD by (1) decreasing the chemotaxis of leukocytes, and (2) inhibiting the formation of biologically active products from oxidized LDL. In some embodiments, the modulator of MIF also decreases any undesired inflammation resulting from administration of the Lp-PLA2 antagonist.

In some embodiments, the modulator of MIF inhibits inflammation and treats a cardiovascular disorder by modulating MIF. In some embodiments, the cardiovascular disorder agent is a leukotriene (e.g., LTA4, LTB4, LTC4, LTD4, LTE4, and LTF4) inhibitor (e.g., an antagonist of 5-LO, FLAP, LTA4H, LTA4S, LTA4R; LTB4R; LTB4R1, LTB4R2, LTC4S, LTC4R, LTD4R, LTE4R, CYSLTR1, or CYSLTR2). In some embodiments, the second active agent is an antagonist of 5-LO. In some embodiments, the second active agent is an antagonist of FLAP. In some embodiments, the second active agent is A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio)-α,α-dimethyl-5-(1-methylethyl)-1H-indole-2-propanoic acid, sodium salt); MK591 (341-4[(4-chlorophenyl)methyl]-3-[(t-butylthio)-5-((2-quinoly)methoxy)-1H-indol-2]-, dimethylpropanoic acid); RP64966 ([4-[5-(3-Phenyl-propyl)thiophen-2-yl]butoxy]acetic acid); SA6541 ((R)-S-[[4-(dimethylamino)phenyl]methyl]-N-(3-mercapto-2-methyl-1-oxopropyl-L-cycleine); SC-56938 (ethyl-14244-(phenylmethyl)phenoxy]ethyl]-4-piperidine-carboxylate); VIA-2291 (Via Pharmaceuticals); WY-47,288 (24(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); or combinations thereof. In some embodiments, the modulator of MIF (i.e., a MIF antagonist) and a leukotriene antagonist synergistically treat a CVD by (1) decreasing the chemotaxis of leukocytes, and (2) inhibiting the adhesion and activation of leukocytes on the endothelium, decreasing the chemotaxis of neutrophils and reducing the formation of reactive oxygen species. In some embodiments, the modulator of MIF also decreases any undesired inflammation resulting from administration of the leukotriene-antagonist.

### Gene Therapy

In some embodiments, are methods and pharmaceutical compositions for modulating a disorder of a cardiovascular system, comprising a synergistic combination of (a) a therapeutically-effective amount of a modulator of MIF; and (b) gene therapy.

In some embodiments, the gene therapy comprises modulating the concentration of a lipid and/or lipoprotein (e.g., HDL) in the blood of an individual in need thereof. In some embodiments, modulating the concentration of a lipid and/or lipoprotein (e.g., HDL) in the blood comprises transfecting DNA into an individual in need thereof. In some embodiments, the DNA encodes an Apo A1 gene, an LCAT gene, and/or an LDL gene. In some embodiments, the DNA is transfected into a liver cell.

In some embodiments, the DNA is transfected into a liver cell via use of ultrasound. For disclosures of techniques related to transfecting ApoA1 DNA via use of ultrasound see U.S. Pat. No. 7,211,248, which is hereby incorporated by reference for those disclosures.

In some embodiments, an individual is administered a vector engineered to carry the human gene (the “gene vector”). For disclosures of techniques for creating an LDL gene vector see U.S. Pat. No. 6,784,162, which is hereby incorporated by reference for those disclosures. In some embodiments, the gene vector is a retrovirus. In some embodiments, the gene vector is not a retrovirus (e.g. it is an adenovirus; a lentivirus; or a polymeric delivery system such as METAFECTENE, SUPERFECT®, EFFECTENE®, or MIRUS TRANSIT). In certain instances, a retrovirus, adenovirus, or lentivirus will have a mutation such that the virus is rendered incompetent.

In some embodiments, the vector is administered in vivo (i.e., the vector is injected directly into the individual, for example into a liver cell), ex vivo (i.e., cells from the individual are grown in vitro and transduced with the gene vector, embedded in a carrier, and then implanted in the individual), or a combination thereof.

In certain instances, after administration of the gene vector, the gene vector infects the cells at the site of administration (e.g. the liver). In certain instances the gene sequence is incorporated into the subject's genome (e.g. when the gene vector is a retrovirus). In certain instances the therapy will need to be periodically re-administered (e.g. when the gene vector is not a retrovirus). In some embodiments, the therapy is re-administered annually. In some embodiments, the therapy is re-administered semi-annually. In some embodiments, the therapy is re-administered when the subject's HDL level decreases below about 60 mg/dL. In some embodiments, the therapy is re-administered when the subject's HDL level decreases below about 50 mg/dL. In some embodiments, the therapy is re-administered when the subject's HDL level decreases below about 45 mg/dL. In some embodiments, the therapy is re-administered when the subject's HDL level decreases below about 40 mg/dL. In some embodiments, the therapy is re-administered when the subject's HDL level decreases below about 35 mg/dL. In some embodiments, the therapy is re-administered when the subject's HDL level decreases below about 30 mg/dL.

### RNAi Therapies

In some embodiments, are methods and pharmaceutical compositions for Modulating a disorder of a cardiovascular system, comprising a synergistic combination of (a) a therapeutically-effective amount of a modulator of MIF; and (b) silencing the expression of a gene that increases the concentration of a lipid in blood (the “target gene”). In some embodiments, the target gene is Apolipoprotein B (Apo B), Heat Shock Protein 110 (Hsp 110), and Proprotein Convertase Subtilisin Kexin 9 (Pcsk9).

In some embodiments, the target gene is silenced by RNA interference (RNAi). In some embodiments, the RNAi therapy comprises use of an siRNA molecule. In some embodiments, a double stranded RNA (dsRNA) molecule with sequences complementary to an mRNA sequence of a gene to be silenced (e.g., Apo B, Hsp 110 and Pcsk9) is generated (e.g. by PCR). In some embodiments, a 20-25 by siRNA molecule with sequences complementary to an mRNA sequence of a gene to be silenced is generated. In some embodiments, the 20-25 by siRNA molecule has 2-5 by overhangs on the 3′ end of each strand, and a 5′ phosphate terminus and a 3′ hydroxyl terminus. In some embodiments, the 20-25 by siRNA molecule has blunt ends. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to, herein as “Sambrook”); Current Protocols in Molecular Biology (F. M. Ausubel et al., eds, 1987, including supplements through 2001); Current. Protocols in Nucleic Acid Chemistry John Wiley & Sons, Inc., New York, 2000) which are hereby incorporated by reference for such disclosure.

In some embodiments, an siRNA molecule is “fully complementary” (i.e., 100% complementary) to the target gene. In some embodiments, an antisense molecule is “mostly complementary” (e.g., 99%, 98%, 97%, 96%, 95%, 94%, 93%, 92%, 91%, 90%, 85%, 80%, 75%, or 70% complementary) to the target gene. In some embodiments, there is a 1 by mismatch, a 2 by mismatch, a 3 by mismatch, a 4 by mismatch, or a 5 by mismatch.

In certain instances, after administration of the dsRNA or siRNA molecule, cells at the site of administration (e.g. the cells of the liver and/cm-small intestine) are transformed with the dsRNA or siRNA molecule. In certain instances following transformation, the dsRNA molecule is cleaved into multiple fragments of about 20-25 by to yield siRNA molecules. In certain instances, the fragments have about 2 bp overhangs on the 3′ end of each strand.

In certain instances, an siRNA molecule is divided into two strands (the guide strand and the anti-guide strand) by an RNA-induced Silencing Complex (RISC). In certain instances, the guide strand is incorporated into the catalytic component of the RISC. (i.e. argonaute). In certain instances, the guide strand binds to a complementary RBI mRNA sequence. In certain instances, the RISC cleaves an mRNA sequence of a gene to be silenced. In certain instances, the expression of the gene to be silenced is down-regulated.

In some embodiments, a sequence complementary to an mRNA sequence of a target gene is incorporated into a vector. In some embodiments, the sequence is placed between two promoters. In some embodiments, the promoters are orientated in opposite directions. In some embodiments, the vector is contacted with a cell. In certain instances, a cell is transformed with the vector. In certain instances following transformation, sense and anti-sense strands of the sequence are generated. In certain instances, the sense and anti-sense strands hybridize to forme dsRNA molecule which is cleaved into siRNA molecules. In certain instances, the strands hybridize to form an siRNA molecule. In some embodiments, the vector is a plasmid (e.g. pSUPER; pSUPER.neo; pSUPER.neo+gfp).

In some embodiments, an siRNA molecule is administered in vivo (i.e., the vector is injected directly into the individual, for example into a liver cell or a cell of the small intestine, or into the blood stream).

In some embodiments, a siRNA molecule is formulated with a delivery vehicle (e.g., a liposome, a biodegradable polymer, a cyclodextrin, a PLGA microsphere, a PLCA microsphere, a biodegradable nanocapsule, a bioadhesive microsphere, or a proteinaceous vector), carriers and diluents, and other pharmaceutically-acceptable excipients. For methods of formulating and administering a nucleic acid molecule to an individual in need thereof see Akhtar et al., 1992, Trends Cell Bio., 2, 139; Delivery Strategies for Antisense Oligonucleotide Therapeutics, ed. Akhtar, 1995; Maurer et al., 1999, Mol. Membr. Biol., 16, 129-140; Hofland and Huang, 1999, Handb. Exp. Pharmacol., 137, 165-192; Lee et al., 2000, ACS Syrnp. Ser., 752, 184-192; Beigelman et al., U.S. Pat. No. 6,395,713; Sullivan et al., PCT WO 94/02595; Gonzalez et al., 1999, Bioconjugate Chem., 10, 1068-1074; Wang et al., International PCT publication Nos. WO 03/47518 and WO 03/46185; U.S. Pat. No. 6,447,796; US Patent Application Publication No. US 2002130430; O'Hare and Normand, International PCT Publication No. WO 00/53722; and U.S. Patent Application Publication No. 20030077829; U.S. Provisional patent application No. 60/678,531, all of which are hereby incorporated by reference for such disclosures.

In some embodiments, an siRNA molecule described herein is administered to the liver by any suitable manner (see e.g., Wen et al., 2004, World J Gastroenterol., 10, 244-9; Murao et al., 2002, Pharm Res., 19, 1808-14; Liu et al., 2003, Gene Ther., 10, 180-7; Hong et al., 2003, 3 Pharm. Pharmacol. 54, 51-8; Herrmann et al., 2004, Arch Virol., 149, 1611-7; and Matsuno et al., 2003, Gene Ther., 10, 1559-66).

In some embodiments, an siRNA molecule described herein is administered iontophoretically, for example to a particular organ or compartment. (e.g., the liver or small intestine). Non-limiting examples of iontophoretic delivery are described in, for example, WO 03/043689 and WO 03/030989, which are hereby incorporated by reference for such disclosures.

In some embodiments, an siRNA molecule described herein is administered systemically (i.e., in vivo systemic absorption or accumulation of an siRNA molecule in the blood stream followed by distribution throughout the entire body). Administration routes contemplated for systemic administration include, but are not limited to, intravenous, subcutaneons, portal vein, intraperitoneal, and intramuscular. Each of these administration routes exposes the siRNA molecules of the invention to an accessible diseased tissue (e.g., liver).

In certain instances the therapy will need to be periodically re-administered. In some embodiments, the therapy is re-administered annually. In some embodiments, the therapy is re-administered semi-annually. In some embodiments, the therapy is administered monthly. In some embodiments, the therapy is administered weekly. In some embodiments, the therapy is re-administered when the subject's HDL level decreases below about 60 mg/dL. In some embodiments, the therapy is re-administered when the subject's HDL level decreases below about 50 mg/dL. In some embodiments, the therapy is re-administered when the subject's HDL level decreases below about 45 mg/dL. In some embodiments, the therapy is re-administered when the subject's HDL level decreases below about 40 mg/dL. In some embodiments, the therapy is re-administered when the subject's HDL level decreases below about 35 mg/dL. In some embodiments, the therapy is re-administered when the subject's HDL level decreases below about 30 mg/dL.

For disclosures of techniques related to silencing the expression of Apo B and/or Hsp110 see U.S. Pub. No. 2007/0293451 which is hereby incorporated by reference for such disclosures. For disclosures of techniques related to silencing the expression of Pcsk9 see U.S. Pub. No. 2007/0173473, which is hereby incorporated by reference for such disclosures.

### Antisense Therapies

In some embodiments, are methods and pharmaceutical compositions for modulating a disorder of a cardiovascular system, comprising a synergistic combination of (a) a therapeutically-effective amount of a modulator of MIF; and (b) inhibiting the expression, of and/or activity of an RNA sequence that increases the concentration of a lipid in blood (the “target sequence”). In some embodiments, inhibiting the expression of and/or activity of a target sequence comprises use of an antisense-molecule complementary to the target sequence. In some embodiments, the target sequence is MicroRNA-122 (miRNA-122 or mRNA-122). In certain instances, inhibiting the expression of and/or activity of miRNA-122 results (partially or fully) in a decrease in the concentration of cholesterol and/or lipids in blood.

In some embodiments, an antisense molecule that is complementary to a target sequence is generated (e.g. by PCR). In some embodiments, the antisense molecule is about 15 to about 30 nucleotides. In some embodiments, the antisense molecule is about 17 to about 28 nucleotides. In some embodiments, the antisense molecule is about 19 to about 26 nucleotides. In some embodiments, the antisense molecule is about 21 to about 24 nucleotides. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); Current Protocols in Nucleic Acid Chemistry John Wiley & Sons, Inc., New York, 2000) which are hereby incorporated by reference for such disclosure.

In some embodiments, the antisense molecules are single-stranded, double-stranded, circular or hairpin. In some embodiments, the antisense molecules contain structural elements (e.g., internal or terminal bulges, or loops).

In some embodiments, an antisense molecule is “fully complementary” (i.e., 100% complementary) to the target sequence. In some embodiments, an antisense molecule is “mostly complementary” (e.g., 99%, 98%, 97%, 96%, 95%, 94%, 93%, 92%, 91%, 90%, 85%, 80%, 75%, or 70% complementary) to the target RNA sequence. In some embodiments, there is a 1 by mismatch, a 2 by mismatch, a 3 by mismatch, a 4 by mismatch, or a 5 by mismatch.

In some embodiments, the antisense molecule hybridizes to the target sequence. As used herein, “hybridize” means the pairing of nucleotides of an antisense molecule with corresponding nucleotides of the target sequence. In certain instances, hybridization involves the formation of one or more hydrogen bonds (e.g., Watson-Crick, Hoogsteen or reversed Hoogsteen hydrogen bonding) between the pairing nucleotides.

In certain instances, hybridizing results (partially or fully) in the degradation, cleavage, and/or sequestration of the RNA sequence.

In some embodiments, a siRNA molecule is formulated with a delivery vehicle (e.g., a liposome, a biodegradable polymer, a cyclodextrin, a PLGA microsphere, a PLCA microsphere, a biodegradable nanocapsule, a bioadhesive microsphere, or a proteinaceous vector), carriers and diluents, and other pharmaceutically acceptable excipients. For methods of formulating and administering a nucleic acid molecule to an individual in need thereof see Akhtar et al., 1992. Trends Cell Bio., 2, 139; Delivery Strategies for Antisense Oligonucleotide Therapeutics, ed. Akhtar, 1995; Maurer et al., 1999, Mol. Membr. Biol., 16, 129-140; Hofland and Huang, 1999, Handb. Exp. Pharmacol., 137, 165-192; Lee et al., 2000, ACS Symp. Ser., 752, 184-192; Beigelman et al., U.S. Pat. No. 6,395,713; Sullivan et al., PCT WO 94/02595; Gonzalez et al., 1999, Bioconjugate Chem., 10, 1068-1074; Wang et al., International PCT publication Nos. WO 03/47518 and WO 03/46185; U.S. Pat. No. 6,447,796; US Patent Application Publication No. US 2002130430; O'Hare and Normand, International PCT Publication No. WO 00/53722; and U.S. Patent Application Publication No. 20030077829; U.S. Provisional patent application No. 60/678,531, all of which are hereby incorporated by reference for such disclosures.

In some embodiments, an siRNA Molecule described herein is administered to the liver by any suitable manner (see e.g., Wen et al., 2004, World J Gastroenterol., 10, 244-9; Murao et al., 2002, Pharm Res., 19, 1808-14; Liu et al., 2003, Gene Ther 10, 180-7; Hong et al., 2003, J. Pharm Pharmacol., 54, 51-8; Herrmann et al., 2004, Arch Virol., 149, 1611-7; and Matsuno et al., 2003, Gene Ther., 10, 1559-66).

In some embodiments, an siRNA molecule described herein is administered iontophoretically, for example to a particular organ or compartment (e.g., the liver or small intestine). Non-limiting examples of iontophoretic delivery are described in, for example, WO 03/043689 and WO 03/030989, which are hereby incorporated by reference for such disclosures.

In some embodiments, an siRNA molecule described herein is administered systemically (i.e., in vivo systemic absorption or accumulation of an siRNA molecule in the blood stream followed by distribution throughout the entire body). Administration routes contemplated for systemic administration include, but are not limited to, intravenous, subcutaneous, portal vein, intraperitoneal, and intramuscular. Each of these administration routes exposes the siRNA molecules of the invention to an accessible diseased tissue (e.g., liver).

In certain instances the therapy will need to be periodically re-administered. In some embodiments, the therapy is re-administered annually. In some embodiments, the therapy is re-administered semi-annually. In some embodiments, the therapy is administered monthly. In some embodiments, the therapy is administered weekly. In some embodiments, the therapy is re-administered when the subject's HDL level decreases below about 60 mg/dL. In some embodiments, the therapy is re-administered when the subject's HDL level decreases below about 50 mg/dL. In some embodiments, the therapy is re-administered when the subject's HDL level decreases below about 45 mg/dL. In some embodiments, the therapy is re-administered when the subject's HDL level decreases below about 40 mg/dL. In some embodiments, the therapy is re-administered when the subject's HDL level decreases below about 35 mg/dL. In some embodiments, the therapy is re-administered when the subject's HDL level decreases below about 30 mg/dL.

For disclosures of techniques related to silencing the expression of miRNA-122 see WO 071027775A2, which is hereby incorporated by reference for such disclosures.

### Device-Mediated Therapies

In some embodiments, the device mediated strategy comprises removing a lipid from an HDL molecule in an individual in need thereof (delipidation), removing an LDL molecule from the blood or plasma of an individual in need thereof (delipidation), or a combination thereof. For disclosures of techniques for removing a lipid from an HDL, molecule and removing an LDL Molecule from the blood or plasma of an individual in need thereof see U.S. Pub. No. 2008/0230465, which is hereby incorporated by reference for those disclosures.

In certain instances, the delipidation therapy will need to be periodically re-administered. In some embodiments, the delipidation therapy is re-administered annually. In some embodiments, the delipidation therapy is re-administered semi-annually. In some embodiments, the delipidation therapy is re-administered monthly. In some embodiments, the delipidation therapy is re-administered semi-weekly. In some embodiments, the therapy is re-administered when the subject's HDL level decreases below about 60 mg/dL. In some embodiments, the therapy is re-administered when the subject's. HDL level decreases below about 50 mg/dL. In some embodiments, the therapy is re-administered when the subject's HDL level decreases below about 45 mg/dL. In some embodiments, the therapy is re-administered when the subject's HDL level decreases below about 40 mg/dL. In some embodiments, the therapy is re-administered when the subject's HDL level decreases below about 35 mg/dL. In some embodiments, the therapy is re-administered when the subject's HDL level decreases below about 30 mg/dL.

### VI. Pharmaceutical Compositions

Disclosed herein, in certain embodiments, is a pharmaceutical composition for modulating a disorder of a cardiovascular system, comprising a synergistic combination of (a) a therapeutically-effective amount of a modulator of MIF; and (b) a second active agent selected from an agent that treats cardiovascular disorders. In some embodiments, the agent that treats cardiovascular disorders induces undesired inflammation.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, the pharmaceutical composition for modulating a disorder of a cardiovascular system further comprises a pharmaceutically acceptable diluent(s), excipient(s), or carrier(s). In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions described herein are formulated into any suitable dosage form, including but not limited to, aqueous oral dispersions, liquids, gels, syrups, elixirs, slurries, suspensions and the like, for oral ingestion by a individual to be treated, solid oral dosage forms, aerosols, controlled release formulations, fast melt formulations, effervescent formulations, lyophilized formulations, tablets, powders, pills, dragees, capsules, modified release formulations, delayed release formulations, extended release formulations, pulsatile, release formulations, multiparticulate formulations, and mixed immediate release and controlled release formulations.

### Multi-Particulate Dosage Forms

In some embodiments, the pharmaceutical compositions described herein are formulated as mulitparticulate formulations. In some embodiments, the pharmaceutical compositions described herein comprise a first population of particles and a second population of particles. In some embodiments, the first population comprises an active agent. In some embodiments, the second population comprises an active agent. In some embodiments, the dose of active agent in the first population is equal to the dose of active agent in the second population. In some embodiments, the dose of active agent in the first population is not equal to (e.g., greater than or less than) the dose of active agent in the second population.

In some embodiments, the active agent of the first population is released before the active agent of the second population. In some embodiments, the second population of particles comprises a modified-release (e.g., delayed-release, controlled-release, or extended release) coating. In some embodiments, the second population of particles comprises a modified-release (e.g., delayed-release, controlled-release, or extended release) matrix.

Coating materials for use with the pharmaceutical compositions described herein include, but are not limited to, polymer coating materials (e.g., cellulose acetate phthalate, cellulose acetate trimaletate, hydroxy propyl methylcellulose phthalate, polyvinyl acetate phthalate); ammonio methacrylate copolymers (e.g., Eudragit® RS and RL); poly acrylic acid and poly acrylate and methacrylate copolymers (e.g., Eudragite S and L, polyvinyl acetaldiethylamino acetate, hydroxypropyl methylcellulose acetate succinate, shellac); hydrogels and gel-forming materials (e.g., carboxyvinyl polymers, sodium alginate, sodium carmellose, calcium carmellose, sodium carboxymethyl starch, poly vinyl alcohol, hydroxyethyl cellulose, methyl cellulose, gelatin, starch, hydroxypropyl cellulose, hydroxypropyl methylcellulose, polyvinylpyrrolidone, crosslinked starch, microcrystalline cellulose, chitin, aminoacryl-methacrylate copolymer, pullulan, collagen, casein, agar, gum arabic, sodium carboxymethyl cellulose, (swellable hydrophilic polymers) poly(hydroxyalkyl methacrylate) (m. wt. ^{˜}5 k-5,000 k), polyvinylpyrrolidone (m. wt. ^{˜}10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. ^{˜}30 k-300 k), polysaccharides, such as agar, acacia, karaya, tragacanth, algins and guar, polyacrylamides, Polyox® polyethylene oxides (m. wt. ^{˜}10 k-5,000 k), AquaKeep® acrylate polymers, diesters of polyglucan, crosslinked polyvinyl alcohol and poly N-vinyl-2-pyrrolidone sodium starch; hydrophilic polymers (e.g., polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid, other acrylic acid derivatives, sorbitan esters, natural gums, lecithins, pectin, alginates, ammonia alginate, sodium, calcium, potassium alginates, propylene glycol alginate, agar, arabic gum, karaya gum, locust bean gum, tragacanth gum, carrageens gum, guar gum, xanthan gum, scleroglucan gum); or combinations thereof. In some embodiments, the coating comprises a plasticiser, a lubricant, a solvent, or combinations thereof. Suitable plasticisers include, but are not limited to, acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

In some embodiments, the second population of particles comprises a modified release matrix material. Materials for use with the pharmaceutical compositions described herein include, but are not limited to microcrystalline cellulose, sodium carboxymethylcellulose, hydroxyalkylcelluloses (e.g., hydroxypropylmethylcellulose and hydroxypropylcellulose), polyethylene oxide, alkylcelluloses (e.g., methylcellulose and ethylcellulose), polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate, or combinations thereof.

In some embodiments, the first population of particles comprises a cardiovascular disorder agent. In some embodiments, the second population of particles comprises a (1) a modulator of MIF; (2) a Modulator of an interaction between RANTES and Platelet Factor 4; or (3) combinations thereof. In some embodiments, the first population of particles comprises a (1) a modulator of MIF; (2) a modulator of an interaction between RANTES and Platelet Factor 4; or (3) combinations thereof. In some embodiments, the second population of particles comprises a cardiovascular disorder agent.

### Additional Dosage Forms

Dragee cores are provided with suitable coatings. For this purpose, concentrated sugar solutions, are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active agent doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle Capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations disclosed herein are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a MIF receptor; inhibitor; include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures, thereof such as E461, Ethocel®, Aqualone-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, not limited to, pharmaceutically acceptable aqueous oral dispersions, emulsions, solutions, elixirs, gels, and Syrups. See, e.g., Singh et al., Encyclopedia of Pharmaceutical Technology, 2nd Ed., pp. 754-757 (2002). In addition to a MIF receptor inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are elf-emulsifying drug delivery systems (SEDDS). Emulsions aredispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous-phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the pharmaceutical compositions disclosed herein are optionally in a form of an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve, to deliver a metered amount. Capsules and cartridges of such as, by way of example only, gelatin for use in an inhaler or insufflator are formulated containing a powder mix and a suitable powder base such as lactose or starch.

Buccal formulations include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of a pharmaceutical compositions disclosed here are administered for example, by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) an active agent; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery is optionally accomplished by means of iontophoretic patches and the like. Additionally, transdermal patches provide controlled delivery. The rate of absorption is optionally slowed by using rate-controlling membranes or by trapping an active agent within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing an active agent optionally with carriers, optionally a rate controlling barrier to deliver a an active agent to the skin of the host at a controlled and predetermined rate over a prolonged period of time, and means to secure the device to the skin.

Formulations suitable for intramuscular, subcutaneous, or intravenous injection include physiologically acceptable sterile aqueous or non-aqueous solutions, dispersions, suspensions or emulsions, and sterile powders for reconstitution into sterile injectable-solutions or dispersions. Examples of suitable aqueous and non-aqueous, carriers, diluents, solvents, or vehicles including water, ethanol, polyols (propyleneglycol, polyethylene, glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing, agents. Pharmaceutical formulations for parenteral administration include aqueous solutions of an active agent in water soluble form. Additionally; suspensions are optionally prepared as appropriate oily injection suspensions.

In some embodiments, an active agent disclosed herein is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

An active agent disclosed herein is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

### VII. Dosages and Administration

In some embodiments, the pharmaceutical compositions disclosed herein are administered to an individual in need thereof. In some embodiments, the pharmaceutical compositions disclosed herein are administered to an individual diagnosed with (i.e., satisfies the diagnostic criteria for) a cardiovascular disease (e.g., atherosclerosis, angina, stenosis, restenosis, high blood pressure, an aneurysm, an embolism, a blood clot, and/or an infarction (e.g., a myocardial infarction or stroke). In some embodiments, the pharmaceutical compositions disclosed herein are administered to an individual suspected of having a cardiovascular disease. In some embodiments, the pharmaceutical compositions disclosed herein are administered to an individual predisposed to develop a cardiovascular disease.

In certain instances, an individual is at risk of atherosclerosis if their c-reactive protein (CRP) levels are above about 3.0 mg/L. In certain instances, an individual is at risk of atherosclerosis if their homocysteine levels exceed about 15.9 mmol/L. In certain instances, an individual is at risk of atherosclerosis if their LDL levels exceed about 160 mg/dL. In certain instances, an individual is at risk of atherosclerosis if their HDL levels are below about 40 mg/dL. In certain instances, an individual is at risk of atherosclerosis if their serum creatinine levels exceed about 1.5 mg/dL. In certain instances, an individual is pre-disposed to develop atherosclerosis if they possess the “G” allele of SNP rs10757278 and/or the “C” allele of SNP rs1333049 both of which are located at the locus 9p21. For disclosures regarding the “G” allele of SNP rs10757278 and/or the “C” allele of SNP rs1333049 see Science, Jun. 8, 2007; 316(5830):1491-93 which is herein incorporated by reference for such disclosures. In certain instances, an individual is pre-disposed to develop atherosclerosis if they possess LTA4H haplotypes Hap A, HapB, HapC, HapL, HapK, and/or HapQ. For disclosures regarding LTA4H haplotypes see International Publication No. WO/2006/105439 which is herein incorporated by reference for such disclosures.

The daily dosages appropriate for an active agent disclosed herein are from about 0.01 to 3 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages are optionally altered depending on anumber of variables, not limited to the activity of the active agents used, the diseases or conditions to be treated, the mode of administration, the requirements of the individual subject, the severity of the disease or condition being treated, and the judgment of the practitioner.

In some embodiments, administration of the cardiovascular disorder agent results in (either partially or fully) undesired inflammation. In some embodiments, the anti-inflammatory agent is administered to the individual to treat the undesired inflammation. In some embodiments, the administration of the cardiovascular agent is discontinued until the inflamed cells and/or tissue is no longer inflamed. In some embodiments, after the inflamed cells and/or tissue are no longer inflamed, administration of the cardiovascular disorder agent recommences. In some embodiments, administration of the cardiovascular agent recommences in combination with an alternative dose of the anti-inflammatory agent.

In the case wherein the individual's condition does not improve, upon the doctor's discretion the administration of an active agent disclosed herein is optionally administered chronically, that is, for an extended period of time, including throughout the duration of the individual's life in order to ameliorate or otherwise control or limit the symptoms of the individual's disease or condition.

In the case wherein the individual's status does improve, upon the doctor's discretion the administration of an active agent disclosed herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days; 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%; 65%, 70%, 75%, 80%, 85%, 90%, 95%, or 100%.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. An active agent disclosed herein exhibiting high therapeutic indices is preferred. The data obtained from cell culture assays and animal studies are optionally used in formulating a range of dosage for use in human. The dosage of such an active agent disclosed herein lies preferably within a range of circulating concentrations that include the ED50 with minimal toxicity. The dosage optionally varies within this range depending upon the dosage form employed and the route of administration utilized.

### EXAMPLES

### Example 1

### Preparation of Multi-Particulate Dosage Form

A multiparticulate dosage form is prepared. The dosage form comprises an immediate release population of particles containing lovastatin. The dosage form further comprises a controlled-release population of an inhibitor of MIF binding to CXCR2 (as described herein).

10 kg of lovastatin, 23 kg of lactose, 0.7 kg of croscarmellose sodium, 0.7 kg polyvinylpyrrolidone K25 are blended in a high-speed blender. The dry mixture is granulated with 4.3 kg of granulating solution (dissolve 0.02 kg of BHA in 1.7 kg of ethanol while mixing in the high-speed blender and add 2.6 kg of demineralized water to the resulting solution). The granulation is dried in a bed-fluid dryer. The dried granulation is sieved in a 0.5 mm sieve to obtain granulation particles of the desired size.

5-mg of the inhibitor of MIF binding to CXCR2, 26 kg of lactose, 0.8 kg of croscarmellose sodium, 0.8 kg polyvinylpyrrolidone K25 are blended in a high-speed blender. The dry mixture is granulated with 34.3 kg of granulating solution (dissolve 0.02 kg of BHA in 1.7 kg of ethanol while mixing in the high-speed blender and add 2.6 kg of demineralized water to the resulting solution). The granulation is dried in a bed-fluid dryer. The dried granulation is sieved in a 0.5 mm sieve to obtain granulation particles of the desired size. The granules are then sprayed with a controlled release coating composition comprising.

The immediate release granules and the controlled-release granules are mixed together. The resulting mixture is encapsulated in gelatine capsules.

### Example 2

### Toxicity Study Following Statin/Peptide 2 Combination in Mouse Model

### Study Design

Female Harlan Sprague-Dawley mice weighing 20 to 24 g are used. The animals used were within an age range of 6 to 8 weeks at the start of dosing.

The mice are divided into two groups: the experimental group, (n=16) and the control group (n=16). The experimental group receives daily intraperitoneal injections of a combination of simvastatin (80 mg/kg) and an inhibitor of MIF binding to CXCR2 (as described herein) (n=16 mice) for 14 days. The experimental group receives daily intraperitoneal injections of a saline solution (n=16 mice) for 14 days.

The mice are sacrificed for histological studies. Four mice from the experimental group are sacrificed on each of days 5, 7, 12, and 14. Four mice from the control group are sacrificed on each of days 5, 7, 12, and 14.

### Necropsy and Histology

Tissue sample are taken from the (a) heart, (b) kidneys, (c) liver, (d) stomach, and (e) muscle tissues. The sampled muscles tissues are taken from (a) the right fore limb (the biceps femoris, extensor digitorum longus, tibialis cranialis, and vastus medialis); (b) the left hind limb (the biceps brachii, extensor carpi radialis longus, and flexor carpi ulnaris); the abdominal peritoneal; the diaphragm; the masseter superficialis; the tongue; and the trapezius).

Tissues are fixed in buffered 10% formalin, processed to wax blocks, and then sectioned and stained with haematoxylin and eosin for examination by light microscopy. Necrosis is graded subjectively. Minimal necrosis is up to 10 necrotic fibers in the whole section; mild is up to about 20% necrotic fibers; moderate is up to about 50% necrotic fibers; and severe is more than 50% necrotic fibers.

### Electron Microscopy

Samples for ultrastructural assessment are immersion fixed in 2.5% glutaraldehyde fixative. Glutaraldehyde-fixed samples are postfixed in 1% osmium tetroxide and processed to Araldite resin blocks. Thin, 70-90-nm resin sections are cut and stained using uranyl acetate and lead citrate. Ultrastructural morphology is examined with a TEM.

### Muscle Histochemistry

Muscle samples are trimmed, orientated on a cork disk, and frozen in isopentane (Fisher Scientific) pre-cooled with liquid nitrogen. Serial cryosections of 7-um thickness are cut from each sample for fiber typing. Sections are stained for mATPase activity following pre-incubation at high and low pH. One section is placed in an incubating solution at pH 9.4 consisting of 0.5% ATP (Sigma) in 0.1 M glycine/NaCl buffer with 0.75 M CaCl₂ for 45 minutes at 37° C. A further section is pre-incubation in 0.1 M sodium acetate buffer with 10 mM ETDA (pH 4.1-4.3) for 10 minutes at 4° C. before placing in the incubation solution noted previously. Following incubation the slides are transferred to 2% CoCl₂ for 5 minutes followed by 30 seconds in 10% ammonium sulphide solution. Sections are washed thoroughly in distilled water between each step. Sections are lightly counterstained with Carazzi's haematoxylin before being dehydrated, cleared, and mounted in Histomount.

### Muscle Immunohistochemistry

Serial cryostat sections are stained for fast and slow myosin heavy chains using antibodies (e.g., NCL-MHCf for fast myosin heavy chains, and NCL-MHCs for slow myosin heavy chains). The sections are incubated in the primary antibody for 60 minutes, then incubated in the secondary antibody (i.e., rabbit anti-mouse HRP conjugate) for 30 minutes, before being visualized by incubation with 3,3 diaminobenzidine tetrahydrochloride for 5 minutes. All incubations are at room temperature, and sections are washed thoroughly in tris-buffered saline between each step. Sections are counterstained with Carazzi's haematoxylin before being dehydrated, cleared, and mounted in Histomount. Dewaxed sections are subjected to 2 minutes' full pressure in a microwave pressure cooker containing 0.01 M citrate buffer at pH 6.0, and then 5 minutes' digestion at room temperature by proteinase K. Endogenous peroxidase activity is blocked by incubation in a peroxidase inhibitor for 20 minutes, followed by 15 minutes in 20% normal rabbit serum. Mouse monoclonal antibody is applied for 30 minutes, followed by 30 minutes in peroxidase-conjugated rabbit anti-mouse antibody. Vector Laboratory's SG peroxidase substrate kit (SK4700) is then applied for 10 minutes. Following an additional 15 minutes of incubation in 20% normal rabbit serum, a mouse mAB to fast myosin is applied. This is visualized using Vector Red alkaline phosphatase substrate kit (Vector Labs SK5100) for 10 minutes. All incubations were at room temperature, and sections are washed thoroughly in tris-buffered saline between each step. Sections are dehydrated, cleared, and mounted in Histomount.

### Example 3

### Statin/Peptide 2 Combination in Mouse Model of Atherosclerosis

Female ApoE−/− littermate mice 9 to 12 weeks old (The Jackson Lab, Bar Harbor, Me., USA) will serve as the model for atherosclerosis. These are, given a fat-rich diet (21% fat; Altromin C1061) for 12 weeks. During this time, two groups of mice receive thrice weekly intraperitoneal injections of a combination of simvastatin (5 mL/kg) and an inhibitor of MIF binding to CXCR2 as described herein)or a saline solution (n=7 mice).

The mice are sacrificed for histological studies. During, the period of the experiment, the mice are maintained healthy. Blood samples are taken at the start and after the end of the experimental feeding. The leukocyte count is determined by hemocytometry and the sera are collected and the cholesterol level is determined by means of Infinity Cholesterol kits (Thermo Electron, Melbourne, Australia).

The extent of the atherosclerosis is determined at the aortal roots and thoracoabdominal aortas by staining the lipid deposits with oil red 0 stain (Veillard N R, Kwak B, Pelli G, Mulhaupt F, James R W, Proudfoot A E, Mach F. Antagonism of RANTES receptors reduces atherosclerotic plaque formation in mice. Circ Res. 2004; 94: 253-61) and is quantified by means of computerized image analysis (Diskus software, Hilgers, Aachen). Regions of atherosclerotic lesions are determined in 5 micron transverse sections through heart and aortal root. The determination is done for each aortal root by means of lipid-stained regions, of 6 sections, at a distance of 50 um from each other. The regions of atherosclerotic lesions re divided by the entire surface of the valve of each section. The thoracoabdominal aorta is opened along the ventral midline and the regions of lesions re stained in an en face preparation by means of oil red 0 staining. The proportion of lipid deposition is calculated as the stained region divided by the entire thoracoabdominal surface.

### Example 4

### Human Clinical Trial of P4/RANTES Antagonist in Combination with Torcetrapib as a Treatment for Hypercholesterolemia

Study Objective(s): The primary objective of this study is to assess the efficacy of a combination of torcetrapib and an inhibitor of MIF binding to CXCR2 (as described herein) in subjects with homozygous familial hypercholesterolemia (HoFH) versus torcetrapib (60 mg) alone.

### Methods

Study Design: This study is a prospective, double-blind, multicenter, parallel-treatment trial comparing the combination versus torcetrapib alone in male and female subjects ≧18 years of age with HoFH. After initial screening, eligible subjects enter a 4-week screening period, consisting of 2 visits (Weeks-4 and -1), during which all lipid-lowering drugs are discontinued (except for bile acid sequestrants and cholesterol absorption inhibitors) and therapeutic lifestyle change counseling (TLC) according to National Cholesterol Education Program (NCEP) Adult Treatment Panel (ATP-III) clinical guidelines or equivalents initiated. Subjects already on apheresis continue their treatment regimen maintaining consistent conditions and intervals during the study. At Visit 3 (Week 0), subjects begin treatment with the combination fixed combination once daily (QD) for 6 weeks or torcetrapib alone. Final visit (Visit 6) occurs at Week 18. Study visits are timed with subjects' apheresis treatments to occur immediately before the visit procedures, where applicable. When the intervals between aphereses are misaligned with a study drug treatment period, the subjects are kept in the same drug treatment period until the next scheduled apheresis; and until the intervals are brought back to the original length of time. Efficacy measures are done at least 2 weeks after the previous apheresis and just before the apheresis procedure scheduled for the day of study visit:

Number of Subjects: 50 subjects divided into two groups—the experimental group (n=25) and the control group (n=25):

Diagnosis and Main Criteria for Inclusion: Men and women 18 years of age or older with definite evidence of the familial hypercholesterolemia (FH) homozygote per World Health Organization guidelines, and with serum fasting triglyceride (TG)≦400 mg/dL (4.52 mmol/L) for subjects aged>20 years and 200 mg/dL (2.26 mmol/L) for subjects aged 18-20 years, are screened for study participation.

Study Treatment: Subjects are randomized into two groups. During the three 6-week treatment period, subjects in the experimental group take 1 tablet of T/P2 QD, with food, immediately after the morning meal. Subjects in the control group take 1 tablet of T QD, with food, immediately after the morning meal.

Efficacy Evaluations: The primary endpoints are the mean percent changes in HDL-C and LDL-C from baseline to the end of each treatment period (ie, Weeks 6, 12 and 18). A lipid profile which included HDL-C and LDL-C is obtained at each study visit.

Safety Evaluations: Safety is assessed using routine clinical laboratory evaluations (hematology and urinalysis panels at Weeks-4, 0 and 18, and chemistry also at Weeks 6 and 12). Vital signs are monitored at every visit, and physical examinations and electrocardiograms (ECGs) are performed at Weeks 0 and 18. Urine pregnancy testing is carried out at every visit except Week-1. Subjects are monitored for: adverse events (AEs) from Week 0 to Week 18. Week 18 safety assessments are completed at early termination if this took place.

Statistical Methods: The primary efficacy endpoints are the percent changes in HDL-C and LDL-C from baseline to the end of each treatment period (ie, Weeks 6, 12, and 18). The primary efficacy analysis population is the full analysis set (FAS) which includes all subjects who received at least 1 dose of study drug and had both a baseline and at least 1 valid post-baseline measurement at each analysis period.

The primary efficacy endpoints are analyzed through the computation of sample means of percent (or nominal) changes, their 95% confidence intervals (CIs), 1-sample t-test statistics, and corresponding p-values. Incremental treatment differences between different dose levels are also estimated and 95% CIs obtained. Hypothesis testing is 2-sided with an overall family-wise type I error rate of 5% (ie, p=0.05 significance level). Hochberg's procedure is used to control the family-wise error rate for multiple comparisons.

### Example 5

### Human Clinical Trial of MIF Antagonist in Combination with, Atorvastatin as a Treatment for Atherosclerosis

Study Objective(s): To measure the effect of 18 months of treatment with lipid lowering treatment (atorvastatin 80-mg daily) versus 8 months of treatment with atorvastatin in combination with an inhibitor of MIF/CXCR2 binding (as described herein) on coronary artery plaque using intravascular ultrasound (IVUS) imaging of the coronary arteries.

### Study Design:

This study is a prospective, double-blind, multicenter, parallel-treatment trial comparing the effects of atorvastatin 80-mg versus atorvastatin in combination with, (80-mg daily) an inhibitor of MIF/CXCR2 binding as measured by IVUS.

The study consists of three phases: (1) subject identification and cardiac catheterization, (2) screening phase to determine eligibility, which includes a 2-week Placebo Run-in Period, and (3) an 18-month, randomized, double-blind treatment phase.

The study includes a total of up to 12 visits (nine required plus three optional) at which safety and/or efficacy assessments are performed: Qualifying IVUS Visit (Cath 1), Screening Visit 1 (SV1), Optional Screening Visits (SV2 and SV3), Randomization Visit (RV), and Clinic Visits for Month 3 (M3), M6, M9, M12, M15, M17 (optional), and M18.

The primary efficacy parameter, is percent change in total plaque (atheroma) volume (TPV) by IVUS.

Secondary efficacy parameters include nominal change in TPV and change in percent plaque (atheroma) volume (PPV).

### Number of Patients:

Approximately 400 subjects (200 subjects per treatment group) are to be enrolled

### Diagnosis and Main Criteria for Inclusion:

Male and female subjects between 30-75 years of age with CAD who have had a coronary catheterization. Precise angiographic inclusion criteria will determine subject eligibility, specifically the presence of at least one obstruction in a major cardiac vessel with at least a 20% luminal diameter narrowing by visual estimation. In addition, subjects must have had a “target vessel” for IVUS interrogation with no more than 50% luminal narrowing throughout a segment that was a minimum of 30 mm in length (the “target segment”). The target vessel must not have undergone previous intervention, nor have been a candidate for intervention at the time of Baseline catheterization. Lipid entry criterion require subjects to have a low-density lipoprotein cholesterol (LDL-C) between 125 and 210 mg/dL following a 4- to 10-week washout period if the subject is taking antihyperlipidemic medication.

### Study Treatment:

Subjects are divided into the groups. The first group (n=200) receives atorvastatin. The second group (n=200) receives atorvastatin in combination with an inhibitor of MIF/CXCR2 binding.

Placebo Run-in Period: Subjects in the two groups are instructed to take two placebo tablets at bedtime each day and return to the Clinic in two weeks for the Randomization Visit. The time between visits during the Placebo Run-in Period is not to exceed 17 days. Subjects are also required to be at least 90% compliant before randomization to the double-blind period.

Double-Blind Period: Subjects in group 1 are instructed to take 80-mg atorvastatin (2×40-mg tablet) and one placebo tablet daily at bedtime each day for 18 months. Subjects in group 2 are instructed to take 80-mg atorvastatin (2×40-mg tablet) in combination with an inhibitor of MIF/CXCR2 binding daily at bedtime each day for 18 months.

### Efficacy Evaluations:

Primary efficacy variable: The percent change in total plaque volume for all slices of anatomically comparable segments of the target coronary artery from Baseline to Month 18 measured by IVUS.

Safety Evaluations: Safety of the treatment is assessed by an evaluation of type, frequency, intensity, and duration of all reported adverse events (AEs), monitoring of laboratory parameters, and changes in vital signs. Data for electrocardiogram (ECG) results and physical examination findings is collected.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a disorder of a cardiovascular system, comprising co-administering to an individual in need thereof a Synergistic combination of (a) a therapeutically-effective amount of a modulator of MIF selected from: (i) an agent that inhibits MIF binding to CXCR2 and CXCR4 and/or inhibits MIF-activation of CXCR2 and CXCR4; or (ii) an agent that inhibits the ability of MIF to form a homomultimer; and (b) a second active agent selected from an agent that treats a cardiovascular disorder.

2. The method of claim 1, wherein the second active agent is niacin; a fibrate; a statin; an apolipoprotein A-1 modulator; an ACAT modulator; a CETP modulator; a glycoprotein IIb/IIIa modulator; a P2Y12 modulator; an Lp-PLA2 modulator; an anti-hypertensive; a leukotriene inhibitor; an 5-LO inhibitor; a FLAP inhibitor; a diuretic; a vasodilator; a beta-blocker; a calcium-channel blocker; a LTA4H inhibitor, a LTA4S inhibitor, a LTC4S inhibitor, or combinations thereof.

3. The method of claim 1, wherein second active agent is selected from atorvastatin; cerivastatin; fluvastatin; lovastatin; mevastatin; pitavastatin; pravastatin; rostivastatin; simvastatin; simvastatin and ezetimibe; lovastatin and niacin, extended-release; atorvastatin and amlodipine besylate; simvastatin and niacin, bezafibrate; ciprofibrate; clofibrate; gemfibrozil; fenofibrate; DF4 (Novartis); DF5 (Bruin Pharmaceuticals); RVX-208 (Resverlogix); avasimibe; pactimibe sulfate (CS-505); CI-1011 (2,6-diisopropylphenyl [(2,4,6-triisopropylphenyl)acetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N-[[4-(2,2-dimethylpropyl)phenyl]methyl]-N-(hepthyl)urea); F-1394 ((1s,2s)-2-[3-(2,2-dimethylpropyl)-3-nonylureido]aminocyclohexane-1-yl 3-[N,(2,2,5,5,-tetramethyl-1,3-dioxane-4-carbonyl)amino]propionate); CP-113818 (N-[2,4-bis(methylthio)-6-methylpyridin-3-yl)-2-(hexylthio)decanoic acid amide); YM-750; torcetrapib; anacetrapid; JTT-705 (Japan Tobacco/Roche); abciximab; eptifibatide; tirofiban; roxifiban; variabilin; XV 459 (N(3)-(2-(3-(4-formamidinophenyl)isoxazolin-5-yl)acetyl)-N(2)-(1-butyloxycarbonyl)-2,3-diaminopropionate); SR 121566A (3-[N-{4-[4-(aminoiminomethyl)phenyl]-1,3-thiazol-2-yl}-N-(1-carboxymethylpiperid-4-yl)aminol, propionic acid, trihydrochloride); FK419 ((S)-2-acetylamino-3[(R)-[1-[3-(piperidin-4-yl) propionyl]piperidin-3-ylcarbonyl]amino]propionic acid trihydrate); clopidogrel; prasdgrel; cangrelor; AZD6140 (AstraZeneca); MRS 2395 (2,2-Dimethyl-propionic acid 3-(2-chloro-6-methylaminopurin-9-yl)-2-(2,2-dimethyl-propionyloxymethyl)-propyl ester); BX 667 (Berlex Biosciences); BX 048 (Berlex Biosciences); darapladib (SB 480848); SB-435-495 (GlaxoSmithKline); SB-222657 (GlaxoSmithKline); SB-253514 (GlaxoSmithKline); A-81834 (3-(3-(1,1-dimethylethylthio-5-(quinoline-2-ylmethoxy)-1-(4-chloromethylphenyl)indole-2-yl)-2,2-dimethylpropionaldehyde oxime-Q-2-acetic acid; AME103 (Amira); AME803 (Amira); atreleuton; CJ-13610 (4-(3-(4-(2-Methyl-imidazol-1-yl)-phenylsulfanyl)-phenyl)-tetrahydro-pyran-4-carboxylic acid amide); DG-031 ((R)-(+)-alpha-cyclopentyl-4-(2-quinolinylmethoxy)-Benzeneacetic acid); DG-051 (DeCode); MK886 (11(4-chlorophenyl)methyl]3-[(1,1-dimethylethyl)thiol-α,α-dimethyl-5-(1-methylethyl)-1H-indole-2-propanoic acid, sodium salt); MK591 (3-(1-4[(4-chlorophenyl)methyl]-31(t-butylthio)-5-((2-quinoly)methoxy)-1H-indole-2]-, dimethylpropanoic acid); RP64966 ([445-(3-Phenyl-propyl)thiophen-2-yl]butoxy]acetic acid); SA6541 ((R)-S-[[4-(dimethylamino)phenyl]methyl]-N-(3-mercapto-2-methyl-1-oxopropyl-L-cycleine); SC-56938 (ethyl-11214-(phenylmethyl)phenoxy]ethyl]-4-piperidine-carboxylate); VIA-2291 (Via Pharmaceuticals); WY-47,288 (2[(1-naphthalenyloxy)methyl]quinoline); zileuton; ZD-2138 (6-((3-fluoro-5-(tetrahydro-4-methoxy-2,4-pyran-4-yl)phenoxy)methyl)-1-methyl-2(1H)-quinlolinone); or combinations thereof.

4. The method of claim 1, wherein the second active agent is administered before, after, or simultaneously with the modulator of inflammation.

5. The method of claim 1, wherein the disorder is hyperlipidemia; hypercholesterolemia; hyperglyceridemia; combined hyperlipidemia; hypolipoproteinemia; hypocholesterolemia; abetlipoproteinemia; Tangier disease; acute coronary syndrome; unstable angina; non-ST segment elevation myocardial infarction; ST segment elevation myocardial infarction; stable angina; Prinzmetal's angina; arteriosclerosis; atherosclerosis; arterialosclerosis; stenosis; restenosis; venous thrombosis; arterial thrombosis; stroke; transient ischemic attack; peripheral vascular disease; coronary artery disease; hypertension; or combinations thereof.

6. A method of treating a lipid disorder, comprising (a) removing a lipid from the blood of an individual in need thereof; and (b) administering a therapeutically-effective amount of a modulator of MIF selected from: (i) an agent that inhibits MIF binding to CXCR2 and CXCR4 and/or inhibits MIF-activation of CXCR2 and CXCR4; or (ii) an agent that inhibits the ability of MIF to form a homomultimer.

7. The method of claim 6, wherein the disorder is hyperlipidemia; hypercholesterolemia; hyperglyceridemia; combined hyperlipidemia; hypolipoproteinemia; hypocholesterolemia; abetlipoproteinemia; Tangier disease; or combinations thereof.

8. A method of treating a lipid disorder, comprising (a) modulating the concentration of a lipid in the blood of an individual in need thereof; and (b) administering a therapeutically-effective amount of a modulator of MIF selected from: (i) an agent that inhibits MIF binding to CXCR2 and CXCR4 and/or inhibits MIF-activation of CXCR2 and CXCR4; or (ii) an agent that inhibits the ability of MIF to form a homomultimer.

9. The method of claim 8, comprising transfecting DNA encoding an Apo A1 gene, an LCAT gene, an LDL gene, or a combination thereof.

10. The method of claim 8, comprising silencing the expression of Apolipoprotein B (Apo B), Heat Shock Protein 110 (lisp 110), Proprotein Convertase Subtilisin Kexin-9 (Pcsk9), or a combination thereof.

11. The method of claim 8, comprising modulating the activity of microRNA-122.

12. The method of claim 8, wherein the disorder is hyperlipidemia; hypercholesterolemia; hyperglyceridemia; combined hyperlipidemia; hypolipoproteinemia; hypocholesterolemia; abetlipoproteinemia; Tangier disease; or combinations thereof.

13. A pharmaceutical composition for modulating a disorder of a cardiovascular system, comprising a synergistic combination of (a) a therapeutically-effective amount of an agent that treats a cardiovascular disorder; and (b) a therapeutically-effective amount of a modulator of MIF selected from: (i) an agent that inhibits MIF binding to CXCR2 and CXCR4 and/or inhibits MIF-activation of CXCR2 and CXCR4; or (ii) an agent that inhibits the ability of MIF to form a homomultimer.

14. The composition of claim 13, wherein the second active agent is niacin; a fibrate; a statin; an apolipoprotein A-1 modulator; an ACAT modulator; a CETP Modulator; glycoprotein IIb/IIIa modulator; a P2Y12 modulator; an Lp-PLA2 modulator; an anti-hypertensive; a leukotriene inhibitor; an 5-LO inhibitor; a FLAP inhibitor; a diuretic; a vasodilator; a beta-blocker; a calcium-channel blocker; a LTA4H inhibitor, a LTA4S inhibitor, a LTC4S inhibitor, or combinations thereof.

15. The composition of claim 13, wherein the second active agent is selected from atorvastatin; cerivastatin; fluvastatin; lovastatin; mevastatin; pitavastatin; pravastatin; rosuvastatin; simvastatin; simvastatin and ezetimibe; lovastatin and niacin, extended-release; atorvastatin and amlodipine besylate; simvastatin and niacin, bezafibrate; ciprofibrate; clofibrate; gemfibrozil; fenofibrate; DF4 (Novartis); DF5 (Bruin Pharmaceuticals); RVX-208 (Resverlogix); avasimibe; pactimibe sulfate (CS-505); CI-1011 (2,6-diisopropylphenyl [(2,4,6-triisopropylphenyl)acetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N[[4-(2,2-dimethylpropyl)phenyl]methyl]-N-(hepthyl)urea); F-1394 ((1s,2s)-243-(2,2-dimethylpropyl)-3-nonylureido]aminocyclohexane-1-yl 3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; torcetrapib; anacetrapid; JTT-705 (Japan Tobacco/Roche); abciximab; eptifibatide; tirofiban; roxifiban; variabilin; XV 459 (N(3)-(2-(3-(4-formamidinophenyl)isoxazolin-5-yl)acetyl)—N(2)-(1-butyloxycarbonyl)-2,3-diaminopropionate); SR 121566A (3-[N-{4-[4-(aminoiminomethyl)phenyl]-1,3-thiazol-2-yl}-N-(1-carboxymethylpiperid-4-yl)amino]propionic acid, trihydrochloride); FK419 ((S)-2-acetylamino-3-[(R)-[1[3-(piperidin-4-yl) propionyl]piperidin-3-ylcarbonyl]amino]propionic acid trihydrate); clopidogrel; prasugrel; cangrelor; AZD6140 (AstraZeneca); MRS 23,95 (2,2-Dimethyl-propionic acid 3-(2-chloro-6-methylaminopurin-9-yl)-2-(2,2-dimethyl-propionyloxymethyl)-propyl ester); BX 667 (Berlex Biosciences); BX 048 (Berlex Biosciences); darapladib (SB 480848); SB-435-495 (GlaxoSmithKline); SB-222657 (GlaxoSmithKline); SB-253514 (GlaxoSmithKline), A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; CJ-13610 (4-(3-(4-(2-Methyl-imidazol-1-yl)-phenylsulfanyl)-phenyl)-tetrahydro-pyran-4-carboxylic acid amide); DG-031 ((R)-(+)-alpha-cyclopentyl-4-(2-quinolinylmethoxy)-Benzeneacetic acid); DG-051 (DeCode); MK886 (1-[(4-chlorophenyl)methyl]3-[(1,1-dimethylethyl)thio)-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)-S-[[4-(dimethylamino)phenyl]methyl]-N-(3-mercapto-2-methyl-1-oxopropyl-L-cycleine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4=methoxy-2H-pyran-4-yl)phenoxy)methyl)-1-methyl-2(1H)-quinlolinone); or combinations thereof.

16. The composition of claim 13, wherein the composition comprises a first population of particles and a second population of particles.

17. The composition of claim 16, wherein the first population of particles is formulated for immediate release.

18. The composition of claim 16, wherein the second population of particles is formulated for controlled release.

19. The composition of claim 16, wherein the first population of particles comprises a therapeutically-effective amount of an agent that treats a cardiovascular disorder.

20. The composition of claim 16, wherein the second population of particles comprises a therapeutically-effective amount of a modulator of MIF.


SEQUENCE LISTING (291 sequences)

SEQ ID NO: 1 - protein, 18 aa, Artificial Sequence
  Description of Artificial Sequence Synthetic peptide
DWFKAFYDKVAEKFKEAF

SEQ ID NO: 2 - protein, 30 aa, Unknown
  Description of Unknown MIF sequence
IGKIGGAQNRSYSKLLCGLLAERLRISPDR

SEQ ID NO: 3 - protein, 16 aa, Unknown
  Description of Unknown MIF sequence
LCGLLAERLRISPDRV

SEQ ID NO: 4 - protein, 43 aa, Artificial Sequence
  Description of Artificial Sequence Synthetic polypeptide
VNTNVPPRASVPDGFLSELTQQLAQATGKPPQYIAVHVVPDQL

SEQ ID NO: 5 - protein, 19 aa, Artificial Sequence
  Description of Artificial Sequence Synthetic peptide
PDQLMAFGGSSEPCALCSL

SEQ ID NO: 6 - protein, 58 aa, Artificial Sequence
  Description of Artificial Sequence Synthetic polypeptide
VNTNVPPRASVPDGFLSELTQQLAQATGKPPQYIAVHVVPDQLMAFGGSSEPCALCSL

SEQ ID NO: 7 - protein, 22 aa, Artificial Sequence
  Description of Artificial Sequence Synthetic peptide
PDQLMAFGGSSEPCALCSLHSI

SEQ ID NO: 8 - protein, 14 aa, Artificial Sequence
  Description of Artificial Sequence Synthetic peptide
LMAFGGSSEPCALC

SEQ ID NO: 9 - protein, 13 aa, Artificial Sequence
  Description of Artificial Sequence Synthetic peptide
LMAFGGSSEPCAL

SEQ ID NO: 10 - protein, 12 aa, Artificial Sequence
  Description of Artificial Sequence Synthetic peptide
LMAFGGSSEPCA

SEQ ID NO: 11 - protein, 11 aa, Artificial Sequence
  Description of Artificial Sequence Synthetic peptide
LMAFGGSSEPC

SEQ ID NO: 12 - protein, 10 aa, Artificial Sequence
  Description of Artificial Sequence Synthetic peptide
LMAFGGSSEP

SEQ ID NO: 13 - protein, 9 aa, Artificial Sequence
  Description of Artificial Sequence Synthetic peptide
LMAFGGSSE

SEQ ID NO: 14 - protein, 8 aa, Artificial Sequence
  Description of Artificial Sequence Synthetic peptide
LMAFGGSS

SEQ ID NO: 15 - protein, 7 aa, Artificial Sequence
  Description of Artificial Sequence Synthetic peptide
LMAFGGS

SEQ ID NO: 16 - protein, 6 aa, Artificial Sequence
  Description of Artificial Sequence Synthetic peptide
LMAFGG

SEQ ID NO: 17 - protein, 13 aa, Artificial Sequence
  Description of Artificial Sequence Synthetic peptide
MAFGGSSEPCALC

SEQ ID NO: 18 - protein, 12 aa, Artificial Sequence
  Description of Artificial Sequence Synthetic peptide
MAFGGSSEPCAL

SEQ ID NO: 19 - protein, 11 aa, Artificial Sequence
  Description of Artificial Sequence Synthetic peptide
MAFGGSSEPCA

SEQ ID NO: 20 - protein, 10 aa, Artificial Sequence
  Description of Artificial Sequence Synthetic peptide
MAFGGSSEPC

SEQ ID NO: 21 - protein, 9 aa, Artificial Sequence
  Description of Artificial Sequence Synthetic peptide
MAFGGSSEP

SEQ ID NO: 22 - protein, 8 aa, Artificial Sequence
  Description of Artificial Sequence Synthetic peptide
MAFGGSSE

SEQ ID NO: 23 - protein, 7 aa, Artificial Sequence
  Description of Artificial Sequence Synthetic peptide
MAFGGSS

SEQ ID NO: 24 - protein, 6 aa, Artificial Sequence
  Description of Artificial Sequence Synthetic peptide
MAFGGS

SEQ ID NO: 25 - protein, 12 aa, Artificial Sequence
  Description of Artificial Sequence Synthetic peptide
AFGGSSEPCALC

SEQ ID NO: 26 - protein, 11 aa, Artificial Sequence
  Description of Artificial Sequence Synthetic peptide
AFGGSSEPCAL

SEQ ID NO: 27 - protein, 10 aa, Artificial Sequence
  Description of Artificial Sequence Synthetic peptide
AFGGSSEPCA

SEQ ID NO: 28 - protein, 9 aa, Artificial Sequence
  Description of Artificial Sequence Synthetic peptide
AFGGSSEPC

SEQ ID NO: 29 - protein, 8 aa, Artificial Sequence
  Description of Artificial Sequence Synthetic peptide
AFGGSSEP

SEQ ID NO: 30 - protein, 7 aa, Artificial Sequence
  Description of Artificial Sequence Synthetic peptide
AFGGSSE

SEQ ID NO: 31 - protein, 6 aa, Artificial Sequence
  Description of Artificial Sequence Synthetic peptide
AFGGSS

SEQ ID NO: 32 - protein, 11 aa, Artificial Sequence
  Description of Artificial Sequence Synthetic peptide
FGGSSEPCALC

SEQ ID NO: 33 - protein, 10 aa, Artificial Sequence
  Description of Artificial Sequence Synthetic peptide
FGGSSEPCAL

SEQ ID NO: 34 - protein, 9 aa, Artificial Sequence
  Description of Artificial Sequence Synthetic peptide
FGGSSEPCA

SEQ ID NO: 35 - protein, 8 aa, Artificial Sequence
  Description of Artificial Sequence Synthetic peptide
FGGSSEPC

SEQ ID NO: 36 - protein, 7 aa, Artificial Sequence
  Description of Artificial Sequence Synthetic peptide
FGGSSEP

SEQ ID NO: 37 - protein, 6 aa, Artificial Sequence
  Description of Artificial Sequence Synthetic peptide
FGGSSE

SEQ ID NO: 38 - protein, 10 aa, Artificial Sequence
  Description of Artificial Sequence Synthetic peptide
GGSSEPCALC

SEQ ID NO: 39 - protein, 9 aa, Artificial Sequence
  Description of Artificial Sequence Synthetic peptide
GGSSEPCAL

SEQ ID NO: 40 - protein, 8 aa, Artificial Sequence
  Description of Artificial Sequence Synthetic peptide
GGSSEPCA

SEQ ID NO: 41 - protein, 7 aa, Artificial Sequence
  Description of Artificial Sequence Synthetic peptide
GGSSEPC

SEQ ID NO: 42 - protein, 6 aa, Artificial Sequence
  Description of Artificial Sequence Synthetic peptide
GGSSEP

SEQ ID NO: 43 - protein, 9 aa, Artificial Sequence
  Description of Artificial Sequence Synthetic peptide
GSSEPCALC

SEQ ID NO: 44 - protein, 8 aa, Artificial Sequence
  Description of Artificial Sequence Synthetic peptide
GSSEPCAL

SEQ ID NO: 45 - protein, 7 aa, Artificial Sequence
  Description of Artificial Sequence Synthetic peptide
GSSEPCA

SEQ ID NO: 46 - protein, 6 aa, Artificial Sequence
  Description of Artificial Sequence Synthetic peptide
GSSEPC

SEQ ID NO: 47 - protein, 8 aa, Artificial Sequence
  Description of Artificial Sequence Synthetic peptide
SSEPCALC

SEQ ID NO: 48 - protein, 7 aa, Artificial Sequence
  Description of Artificial Sequence Synthetic peptide
SSEPCAL

SEQ ID NO: 49 - protein, 6 aa, Artificial Sequence
  Description of Artificial Sequence Synthetic peptide
SSEPCA

SEQ ID NO: 50 - protein, 7 aa, Artificial Sequence
  Description of Artificial Sequence Synthetic peptide
SEPCALC

SEQ ID NO: 51 - protein, 6 aa, Artificial Sequence
  Description of Artificial Sequence Synthetic peptide
SEPCAL

SEQ ID NO: 52 - protein, 6 aa, Artificial Sequence
  Description of Artificial Sequence Synthetic peptide
EPCALC

SEQ ID NO: 53 - protein, 15 aa, Artificial Sequence
  Description of Artificial Sequence Synthetic peptide
QLMAFGGSSEPCALC

SEQ ID NO: 54 - protein, 14 aa, Artificial Sequence
  Description of Artificial Sequence Synthetic peptide
QLMAFGGSSEPCAL

SEQ ID NO: 55 - protein, 13 aa, Artificial Sequence
  Description of Artificial Sequence Synthetic peptide
QLMAFGGSSEPCA

SEQ ID NO: 56 - protein, 12 aa, Artificial Sequence
  Description of Artificial Sequence Synthetic peptide
QLMAFGGSSEPC

SEQ ID NO: 57 - protein, 11 aa, Artificial Sequence
  Description of Artificial Sequence Synthetic peptide
QLMAFGGSSEP

SEQ ID NO: 58 - protein, 10 aa, Artificial Sequence
  Description of Artificial Sequence Synthetic peptide
QLMAFGGSSE

SEQ ID NO: 59 - protein, 9 aa, Artificial Sequence
  Description of Artificial Sequence Synthetic peptide
QLMAFGGSS

SEQ ID NO: 60 - protein, 8 aa, Artificial Sequence
  Description of Artificial Sequence Synthetic peptide
QLMAFGGS

SEQ ID NO: 61 - protein, 7 aa, Artificial Sequence
  Description of Artificial Sequence Synthetic peptide
QLMAFGG

SEQ ID NO: 62 - protein, 6 aa, Artificial Sequence
  Description of Artificial Sequence Synthetic peptide
QLMAFG

SEQ ID NO: 63 - protein, 9 aa, Artificial Sequence
  Description of Artificial Sequence Synthetic peptide
CSSEPCALC

SEQ ID NO: 64 - protein, 12 aa, Artificial Sequence
  Description of Artificial Sequence Synthetic peptide
CFGGSSEPCALC

SEQ ID NO: 65 - protein, 15 aa, Artificial Sequence
  Description of Artificial Sequence Synthetic peptide
CLMAFGGSSEPCALC

SEQ ID NO: 66 - protein, 8 aa, Artificial Sequence
  Description of Artificial Sequence Synthetic peptide
CAFGGSSC

SEQ ID NO: 67 - protein, 13 aa, Artificial Sequence
  Description of Artificial Sequence Synthetic peptide
CLMAFGGSSEPCC

SEQ ID NO: 68 - protein, 12 aa, Artificial Sequence
  Description of Artificial Sequence Synthetic peptide
CAFGGSSEPCAC

SEQ ID NO: 69 - protein, 11 aa, Artificial Sequence
  Description of Artificial Sequence Synthetic peptide
CMAFGGSSEPC

SEQ ID NO: 70 - protein, 10 aa, Artificial Sequence
  Description of Artificial Sequence Synthetic peptide
CGGSSEPCAC

SEQ ID NO: 71 - protein, 9 aa, Artificial Sequence
  Description of Artificial Sequence Synthetic peptide
NVPRASVPD

SEQ ID NO: 72 - protein, 9 aa, Artificial Sequence
  Description of Artificial Sequence Synthetic peptide
VPDGFLSEL

SEQ ID NO: 73 - protein, 9 aa, Artificial Sequence
  Description of Artificial Sequence Synthetic peptide
CFGGSSEPC

SEQ ID NO: 74 - protein, 20 aa, Artificial Sequence
  Description of Artificial Sequence Synthetic peptide
IAVHVVPDQLMAFGGSSEPC

SEQ ID NO: 75 - protein, 20 aa, Artificial Sequence
  Description of Artificial Sequence Synthetic peptide
CLHSIGKIGGAQNRSYSKLL

SEQ ID NO: 76 - protein, 15 aa, Artificial Sequence
  Description of Artificial Sequence Synthetic peptide
PCALLCSLHSIGKIG

SEQ ID NO: 77 - protein, 15 aa, Artificial Sequence
  Description of Artificial Sequence Synthetic peptide
CSLHSIGKIGGAQNR

SEQ ID NO: 78 - protein, 15 aa, Artificial Sequence
  Description of Artificial Sequence Synthetic peptide
IGKIGGAQNRSYSKL

SEQ ID NO: 79 - protein, 16 aa, Artificial Sequence
  Description of Artificial Sequence Synthetic peptide
GAQNRSYSKLLCGLLA

SEQ ID NO: 80 - protein, 15 aa, Artificial Sequence
  Description of Artificial Sequence Synthetic peptide
CGLLAERLRISPDRV

SEQ ID NO: 81 - protein, 15 aa, Artificial Sequence
  Description of Artificial Sequence Synthetic peptide
ERLRISPDRVYINYY

SEQ ID NO: 82 - protein, 14 aa, Artificial Sequence
  Description of Artificial Sequence Synthetic cyclic peptide
LMAFGGSSEPCALC

SEQ ID NO: 83 - protein, 13 aa, Artificial Sequence
  Description of Artificial Sequence Synthetic cyclic peptide
LMAFGGSSEPCAL

SEQ ID NO: 84 - protein, 12 aa, Artificial Sequence
  Description of Artificial Sequence Synthetic cyclic peptide
LMAFGGSSEPCA

SEQ ID NO: 85 - protein, 11 aa, Artificial Sequence
  Description of Artificial Sequence Synthetic cyclic peptide
LMAFGGSSEPC

SEQ ID NO: 86 - protein, 10 aa, Artificial Sequence
  Description of Artificial Sequence Synthetic cyclic peptide
LMAFGGSSEP

SEQ ID NO: 87 - protein, 9 aa, Artificial Sequence
  Description of Artificial Sequence Synthetic cyclic peptide
LMAFGGSSE

SEQ ID NO: 88 - protein, 8 aa, Artificial Sequence
  Description of Artificial Sequence Synthetic cyclic peptide
LMAFGGSS

SEQ ID NO: 89 - protein, 7 aa, Artificial Sequence
  Description of Artificial Sequence Synthetic cyclic peptide
LMAFGGS

SEQ ID NO: 90 - protein, 6 aa, Artificial Sequence
  Description of Artificial Sequence Synthetic cyclic peptide
LMAFGG

SEQ ID NO: 91 - protein, 13 aa, Artificial Sequence
  Description of Artificial Sequence Synthetic cyclic peptide
MAFGGSSEPCALC

SEQ ID NO: 92 - protein, 12 aa, Artificial Sequence
  Description of Artificial Sequence Synthetic cyclic peptide
MAFGGSSEPCAL

SEQ ID NO: 93 - protein, 11 aa, Artificial Sequence
  Description of Artificial Sequence Synthetic cyclic peptide
MAFGGSSEPCA

SEQ ID NO: 94 - protein, 10 aa, Artificial Sequence
  Description of Artificial Sequence Synthetic cyclic peptide
MAFGGSSEPC

SEQ ID NO: 95 - protein, 9 aa, Artificial Sequence
  Description of Artificial Sequence Synthetic cyclic peptide
MAFGGSSEP

SEQ ID NO: 96 - protein, 8 aa, Artificial Sequence
  Description of Artificial Sequence Synthetic cyclic peptide
MAFGGSSE

SEQ ID NO: 97 - protein, 7 aa, Artificial Sequence
  Description of Artificial Sequence Synthetic cyclic peptide
MAFGGSS

SEQ ID NO: 98 - protein, 6 aa, Artificial Sequence
  Description of Artificial Sequence Synthetic cyclic peptide
MAFGGS

SEQ ID NO: 99 - protein, 9 aa, Artificial Sequence
  Description of Artificial Sequence Synthetic cyclic peptide
GSSEPCALC

SEQ ID NO: 100 - protein, 8 aa, Artificial Sequence
  Description of Artificial Sequence Synthetic cyclic peptide
GSSEPCAL

SEQ ID NO: 101 - protein, 7 aa, Artificial Sequence
  Description of Artificial Sequence Synthetic cyclic peptide
GSSEPCA

SEQ ID NO: 102 - protein, 6 aa, Artificial Sequence
  Description of Artificial Sequence Synthetic cyclic peptide
GSSEPC

SEQ ID NO: 103 - protein, 8 aa, Artificial Sequence
  Description of Artificial Sequence Synthetic cyclic peptide
SSEPCALC

SEQ ID NO: 104 - protein, 7 aa, Artificial Sequence
  Description of Artificial Sequence Synthetic cyclic peptide
SSEPCAL

SEQ ID NO: 105 - protein, 6 aa, Artificial Sequence
  Description of Artificial Sequence Synthetic cyclic peptide
SSEPCA

SEQ ID NO: 106 - protein, 7 aa, Artificial Sequence
  Description of Artificial Sequence Synthetic cyclic peptide
SEPCALC

SEQ ID NO: 107 - protein, 6 aa, Artificial Sequence
  Description of Artificial Sequence Synthetic cyclic peptide
SEPCAL

SEQ ID NO: 108 - protein, 6 aa, Artificial Sequence
  Description of Artificial Sequence Synthetic cyclic peptide
EPCALC

SEQ ID NO: 109 - protein, 15 aa, Artificial Sequence
  Description of Artificial Sequence Synthetic cyclic peptide
QLMAFGGSSEPCALC

SEQ ID NO: 110 - protein, 14 aa, Artificial Sequence
  Description of Artificial Sequence Synthetic cyclic peptide
QLMAFGGSSEPCAL

SEQ ID NO: 111 - protein, 13 aa, Artificial Sequence
  Description of Artificial Sequence Synthetic cyclic peptide
QLMAFGGSSEPCA

SEQ ID NO: 112 - protein, 12 aa, Artificial Sequence
  Description of Artificial Sequence Synthetic cyclic peptide
QLMAFGGSSEPC

SEQ ID NO: 113 - protein, 11 aa, Artificial Sequence
  Description of Artificial Sequence Synthetic cyclic peptide
QLMAFGGSSEP

SEQ ID NO: 114 - protein, 10 aa, Artificial Sequence
  Description of Artificial Sequence Synthetic cyclic peptide
QLMAFGGSSE

SEQ ID NO: 115 - protein, 9 aa, Artificial Sequence
  Description of Artificial Sequence Synthetic cyclic peptide
QLMAFGGSS

SEQ ID NO: 116 - protein, 8 aa, Artificial Sequence
  Description of Artificial Sequence Synthetic cyclic peptide
QLMAFGGS

SEQ ID NO: 117 - protein, 7 aa, Artificial Sequence
  Description of Artificial Sequence Synthetic cyclic peptide
QLMAFGG

SEQ ID NO: 118 - protein, 6 aa, Artificial Sequence
  Description of Artificial Sequence Synthetic cyclic peptide
QLMAFG

SEQ ID NO: 119 - protein, 12 aa, Artificial Sequence
  Description of Artificial Sequence Synthetic cyclic peptide
AFGGSSEPCALC

SEQ ID NO: 120 - protein, 11 aa, Artificial Sequence
  Description of Artificial Sequence Synthetic cyclic peptide
AFGGSSEPCAL

SEQ ID NO: 121 - protein, 10 aa, Artificial Sequence
  Description of Artificial Sequence Synthetic cyclic peptide
AFGGSSEPCA

SEQ ID NO: 122 - protein, 9 aa, Artificial Sequence
  Description of Artificial Sequence Synthetic cyclic peptide
AFGGSSEPC

SEQ ID NO: 123 - protein, 8 aa, Artificial Sequence
  Description of Artificial Sequence Synthetic cyclic peptide
AFGGSSEP

SEQ ID NO: 124 - protein, 7 aa, Artificial Sequence
  Description of Artificial Sequence Synthetic cyclic peptide
AFGGSSE

SEQ ID NO: 125 - protein, 6 aa, Artificial Sequence
  Description of Artificial Sequence Synthetic cyclic peptide
AFGGSS

SEQ ID NO: 126 - protein, 11 aa, Artificial Sequence
  Description of Artificial Sequence Synthetic cyclic peptide
FGGSSEPCALC

SEQ ID NO: 127 - protein, 10 aa, Artificial Sequence
  Description of Artificial Sequence Synthetic cyclic peptide
FGGSSEPCAL

SEQ ID NO: 128 - protein, 9 aa, Artificial Sequence
  Description of Artificial Sequence Synthetic cyclic peptide
FGGSSEPCA

SEQ ID NO: 129 - protein, 8 aa, Artificial Sequence
  Description of Artificial Sequence Synthetic cyclic peptide
FGGSSEPC

SEQ ID NO: 130 - protein, 7 aa, Artificial Sequence
  Description of Artificial Sequence Synthetic cyclic peptide
FGGSSEP

SEQ ID NO: 131 - protein, 6 aa, Artificial Sequence
  Description of Artificial Sequence Synthetic cyclic peptide
FGGSSE

SEQ ID NO: 132 - protein, 10 aa, Artificial Sequence
  Description of Artificial Sequence Synthetic cyclic peptide
GGSSEPCALC

SEQ ID NO: 133 - protein, 9 aa, Artificial Sequence
  Description of Artificial Sequence Synthetic cyclic peptide
GGSSEPCAL

SEQ ID NO: 134 - protein, 8 aa, Artificial Sequence
  Description of Artificial Sequence Synthetic cyclic peptide
GGSSEPCA

SEQ ID NO: 135 - protein, 7 aa, Artificial Sequence
  Description of Artificial Sequence Synthetic cyclic peptide
GGSSEPC

SEQ ID NO: 136 - protein, 6 aa, Artificial Sequence
  Description of Artificial Sequence Synthetic cyclic peptide
GGSSEP

SEQ ID NO: 137 - protein, 9 aa, Artificial Sequence
  Description of Artificial Sequence Synthetic cyclic peptide
CSSEPCALC

SEQ ID NO: 138 - protein, 12 aa, Artificial Sequence
  Description of Artificial Sequence Synthetic cyclic peptide
CFGGSSEPCALC

SEQ ID NO: 139 - protein, 10 aa, Artificial Sequence
  Description of Artificial Sequence Synthetic cyclic peptide
CFGGSSEPCC

SEQ ID NO: 140 - protein, 9 aa, Artificial Sequence
  Description of Artificial Sequence Synthetic cyclic peptide
CFGGSSEPC

SEQ ID NO: 141 - protein, 10 aa, Artificial Sequence
  Description of Artificial Sequence Synthetic cyclic peptide
CGSSEPCALC

SEQ ID NO: 142 - protein, 12 aa, Artificial Sequence
  Description of Artificial Sequence Synthetic cyclic peptide
CAFGGSSEPCAC

SEQ ID NO: 143 - protein, 15 aa, Artificial Sequence
  Description of Artificial Sequence Synthetic cyclic peptide
CLMAFGGSSEPCALC

SEQ ID NO: 144 - protein, 8 aa, Artificial Sequence
  Description of Artificial Sequence Synthetic cyclic peptide
CAFGGSSC

SEQ ID NO: 145 - protein, 13 aa, Artificial Sequence
  Description of Artificial Sequence Synthetic peptide
CTNVPRASVPDGC

SEQ ID NO: 146 - protein, 7 aa, Artificial Sequence
  Description of Artificial Sequence Synthetic peptide
CVPRASC

SEQ ID NO: 147 - protein, 18 aa, Artificial Sequence
  Description of Artificial Sequence Synthetic peptide
VNTNVPRASVPDGFLSEL

SEQ ID NO: 148 - protein, 17 aa, Artificial Sequence
  Description of Artificial Sequence Synthetic peptide
NTNVPRASVPDGFLSEL

SEQ ID NO: 149 - protein, 16 aa, Artificial Sequence
  Description of Artificial Sequence Synthetic peptide
TNVPRASVPDGFLSEL

SEQ ID NO: 150 - protein, 15 aa, Artificial Sequence
  Description of Artificial Sequence Synthetic peptide
NVPRASVPDGFLSEL

SEQ ID NO: 151 - protein, 14 aa, Artificial Sequence
  Description of Artificial Sequence Synthetic peptide
VPRASVPDGFLSEL

SEQ ID NO: 152 - protein, 13 aa, Artificial Sequence
  Description of Artificial Sequence Synthetic peptide
PRASVPDGFLSEL

SEQ ID NO: 153 - protein, 12 aa, Artificial Sequence
  Description of Artificial Sequence Synthetic peptide
RASVPDGFLSEL

SEQ ID NO: 154 - protein, 11 aa, Artificial Sequence
  Description of Artificial Sequence Synthetic peptide
ASVPDGFLSEL

SEQ ID NO: 155 - protein, 10 aa, Artificial Sequence
  Description of Artificial Sequence Synthetic peptide
SVPDGFLSEL

SEQ ID NO: 156 - protein, 14 aa, Artificial Sequence
  Description of Artificial Sequence Synthetic peptide
NVPRASVPDGFLSE

SEQ ID NO: 157 - protein, 13 aa, Artificial Sequence
  Description of Artificial Sequence Synthetic peptide
NVPRASVPDGFLS

SEQ ID NO: 158 - protein, 12 aa, Artificial Sequence
  Description of Artificial Sequence Synthetic peptide
NVPRASVPDGFL

SEQ ID NO: 159 - protein, 11 aa, Artificial Sequence
  Description of Artificial Sequence Synthetic peptide
NVPRASVPDGF

SEQ ID NO: 160 - protein, 10 aa, Artificial Sequence
  Description of Artificial Sequence Synthetic peptide
NVPRASVPDG

SEQ ID NO: 161 - protein, 8 aa, Artificial Sequence
  Description of Artificial Sequence Synthetic peptide
NVPRASVP

SEQ ID NO: 162 - protein, 7 aa, Artificial Sequence
  Description of Artificial Sequence Synthetic peptide
VPRASVP

SEQ ID NO: 163 - protein, 6 aa, Artificial Sequence
  Description of Artificial Sequence Synthetic peptide
PRASVP

SEQ ID NO: 164 - protein, 11 aa, Artificial Sequence
  Description of Artificial Sequence Synthetic peptide
VPRASVPDGFL

SEQ ID NO: 165 - protein, 10 aa, Artificial Sequence
  Description of Artificial Sequence Synthetic peptide
VPRASVPDGF

SEQ ID NO: 166 - protein, 9 aa, Artificial Sequence
  Description of Artificial Sequence Synthetic peptide
VPRASVPDG

SEQ ID NO: 167 - protein, 8 aa, Artificial Sequence
  Description of Artificial Sequence Synthetic peptide
VPRASVPD

SEQ ID NO: 168 - protein, 5 aa, Artificial Sequence
  Description of Artificial Sequence Synthetic peptide
VPRAS

SEQ ID NO: 169 - protein, 23 aa, Artificial Sequence
  Description of Artificial Sequence Synthetic peptide
MPMFIVNTNVPRASVPDGFLSEC

SEQ ID NO: 170 - protein, 15 aa, Artificial Sequence
  Description of Artificial Sequence Synthetic peptide
MPMFIVNTNVPRASV

SEQ ID NO: 171 - protein, 15 aa, Artificial Sequence
  Description of Artificial Sequence Synthetic peptide
FIVNTNVPRASVPDG

SEQ ID NO: 172 - protein, 15 aa, Artificial Sequence
  Description of Artificial Sequence Synthetic peptide
NTNVPRASVPDGFLS

SEQ ID NO: 173 - protein, 15 aa, Artificial Sequence
  Description of Artificial Sequence Synthetic peptide
VPRASVPDGFLSELT

SEQ ID NO: 174 - protein, 22 aa, Artificial Sequence
  Description of Artificial Sequence Synthetic peptide
HSIGKIGGAQNRSYSKLLCGLL

SEQ ID NO: 175 - protein, 20 aa, Artificial Sequence
  Description of Artificial Sequence Synthetic peptide
HSIGKIGGAQNRSYSKLLCG

SEQ ID NO: 176 - protein, 18 aa, Artificial Sequence
  Description of Artificial Sequence Synthetic peptide
HSIGKIGGAQNRSYSKLL

SEQ ID NO: 177 - protein, 16 aa, Artificial Sequence
  Description of Artificial Sequence Synthetic peptide
HSIGKIGGAQNRSYSK

SEQ ID NO: 178 - protein, 15 aa, Artificial Sequence
  Description of Artificial Sequence Synthetic peptide
HSIGKIGGAQNRSYS

SEQ ID NO: 179 - protein, 17 aa, Artificial Sequence
  Description of Artificial Sequence Synthetic peptide
IGKIGGAQNRSYSKLLC

SEQ ID NO: 180 - protein, 15 aa, Artificial Sequence
  Description of Artificial Sequence Synthetic peptide
KIGGAQNRSYSKLLC

SEQ ID NO: 181 - protein, 22 aa, Artificial Sequence
  Description of Artificial Sequence Synthetic peptide
GGAQNRSYSKLLCGLLAERLRI

SEQ ID NO: 182 - protein, 20 aa, Artificial Sequence
  Description of Artificial Sequence Synthetic peptide
AQNRSYSKLLCGLLAERLRI

SEQ ID NO: 183 - protein, 18 aa, Artificial Sequence
  Description of Artificial Sequence Synthetic peptide
NRSYSKLLCGLLAERLRI

SEQ ID NO: 184 - protein, 16 aa, Artificial Sequence
  Description of Artificial Sequence Synthetic peptide
SYSKLLCGLLAERLRI

SEQ ID NO: 185 - protein, 15 aa, Artificial Sequence
  Description of Artificial Sequence Synthetic peptide
YSKLLCGLLAERLRI

SEQ ID NO: 186 - protein, 17 aa, Artificial Sequence
  Description of Artificial Sequence Synthetic peptide
GAQNRSYSKLLCGLLAE

SEQ ID NO: 187 - protein, 15 aa, Artificial Sequence
  Description of Artificial Sequence Synthetic peptide
GAQNRSYSKLLCGLL

SEQ ID NO: 188 - protein, 15 aa, Artificial Sequence
  Description of Artificial Sequence Synthetic peptide
QNRSYSKLLCGLLAE

SEQ ID NO: 189 - protein, 14 aa, Artificial Sequence
  Description of Artificial Sequence Synthetic peptide
HSIGKIGGAQNRSY

SEQ ID NO: 190 - protein, 12 aa, Artificial Sequence
  Description of Artificial Sequence Synthetic peptide
HSIGKIGGAQNR

SEQ ID NO: 191 - protein, 11 aa, Artificial Sequence
  Description of Artificial Sequence Synthetic peptide
KIGGAQNRSYS

SEQ ID NO: 192 - protein, 13 aa, Artificial Sequence
  Description of Artificial Sequence Synthetic peptide
GAQNRSYSKLLCG

SEQ ID NO: 193 - protein, 11 aa, Artificial Sequence
  Description of Artificial Sequence Synthetic peptide
GAQNRSYSKLL

SEQ ID NO: 194 - protein, 13 aa, Artificial Sequence
  Description of Artificial Sequence Synthetic peptide
RSYSKLLCGLLAE

SEQ ID NO: 195 - protein, 11 aa, Artificial Sequence
  Description of Artificial Sequence Synthetic peptide
YSKLLCGLLAE

SEQ ID NO: 196 - protein, 43 aa, Artificial Sequence
  Description of Artificial Sequence Synthetic polypeptide
IAVHVVPDQLMAFGGSSEPCALCSLHSIGKIGGAQNRSYSKLL

SEQ ID NO: 197 - protein, 39 aa, Artificial Sequence
  Description of Artificial Sequence Synthetic polypeptide
IAVHVVPDQLMAFGGSSEPCALCSLHSIGKIGGAQNRSY

SEQ ID NO: 198 - protein, 35 aa, Artificial Sequence
  Description of Artificial Sequence Synthetic polypeptide
IAVHVVPDQLMAFGGSSEPCALCSLHSIGKIGGAQ

SEQ ID NO: 199 - protein, 31 aa, Artificial Sequence
  Description of Artificial Sequence Synthetic polypeptide
IAVHVVPDQLMAFGGSSEPCALCSLHSIGKI

SEQ ID NO: 200 - protein, 27 aa, Artificial Sequence
  Description of Artificial Sequence Synthetic peptide
IAVHVVPDQLMAFGGSSEPCALCSLHS

SEQ ID NO: 201 - protein, 23 aa, Artificial Sequence
  Description of Artificial Sequence Synthetic peptide
IAVHVVPDQLMAFGGSSEPCALC

SEQ ID NO: 202 - protein, 19 aa, Artificial Sequence
  Description of Artificial Sequence Synthetic peptide
IAVHVVPDQLMAFGGSSEP

SEQ ID NO: 203 - protein, 15 aa, Artificial Sequence
  Description of Artificial Sequence Synthetic peptide
IAVHVVPDQLMAFGG

SEQ ID NO: 204 - protein, 11 aa, Artificial Sequence
  Description of Artificial Sequence Synthetic peptide
IAVHVVPDQLM

SEQ ID NO: 205 - protein, 39 aa, Artificial Sequence
  Description of Artificial Sequence Synthetic polypeptide
VVPDQLMAFGGSSEPCALCSLHSIGKIGGAQNRSYSKLL

SEQ ID NO: 206 - protein, 35 aa, Artificial Sequence
  Description of Artificial Sequence Synthetic polypeptide
QLMAFGGSSEPCALCSLHSIGKIGGAQNRSYSKLL

SEQ ID NO: 207 - protein, 31 aa, Artificial Sequence
  Description of Artificial Sequence Synthetic polypeptide
FGGSSEPCALCSLHSIGKIGGAQNRSYSKLL

SEQ ID NO: 208 - protein, 27 aa, Artificial Sequence
  Description of Artificial Sequence Synthetic peptide
SEPCALCSLHSIGKIGGAQNRSYSKLL

SEQ ID NO: 209 - protein, 23 aa, Artificial Sequence
  Description of Artificial Sequence Synthetic peptide
ALCSLHSIGKIGGAQNRSYSKLL

SEQ ID NO: 210 - protein, 19 aa, Artificial Sequence
  Description of Artificial Sequence Synthetic peptide
LHSIGKIGGAQNRSYSKLL

SEQ ID NO: 211 - protein, 15 aa, Artificial Sequence
  Description of Artificial Sequence Synthetic peptide
GKIGGAQNRSYSKLL

SEQ ID NO: 212 - protein, 13 aa, Artificial Sequence
  Description of Artificial Sequence Synthetic peptide
IGGAQNRSYSKLL

SEQ ID NO: 213 - protein, 9 aa, Artificial Sequence
  Description of Artificial Sequence Synthetic peptide
QNRSYSKLL

SEQ ID NO: 214 - protein, 8 aa, Artificial Sequence
  Description of Artificial Sequence Synthetic peptide
IGKIGGAQ

SEQ ID NO: 215 - protein, 10 aa, Artificial Sequence
  Description of Artificial Sequence Synthetic peptide
CIGKIGGAQC

SEQ ID NO: 216 - protein, 10 aa, Artificial Sequence
  Description of Artificial Sequence Synthetic cyclic peptide
CIGKIGGAQC

SEQ ID NO: 217 - protein, 15 aa, Artificial Sequence
  Description of Artificial Sequence Synthetic peptide
CRSYSKLLCGLLAEC

SEQ ID NO: 218 - protein, 15 aa, Artificial Sequence
  Description of Artificial Sequence Synthetic cyclic peptide
CRSYSKLLCGLLAEC

SEQ ID NO: 219 - protein, 14 aa, Artificial Sequence
  Description of Artificial Sequence Synthetic peptide
CGLLAERLRISPDR

SEQ ID NO: 220 - protein, 15 aa, Artificial Sequence
  Description of Artificial Sequence Synthetic peptide
CGLLAERLRISPDRC

SEQ ID NO: 221 - protein, 15 aa, Artificial Sequence
  Description of Artificial Sequence Synthetic cyclic peptide
CGLLAERLRISPDRC

SEQ ID NO: 222 - protein, 8 aa, Artificial Sequence
  Description of Artificial Sequence Synthetic peptide
AYFLYQQQ

SEQ ID NO: 223 - protein, 15 aa, Artificial Sequence
  Description of Artificial Sequence Synthetic peptide
QQQGRLDKLTVTGRL

SEQ ID NO: 224 - protein, 15 aa, Artificial Sequence
  Description of Artificial Sequence Synthetic peptide
GRLDKLTVTSQNLQL

SEQ ID NO: 225 - protein, 11 aa, Artificial Sequence
  Description of Artificial Sequence Synthetic peptide
SQNLQLENLRM

SEQ ID NO: 226 - protein, 15 aa, Artificial Sequence
  Description of Artificial Sequence Synthetic peptide
TVTGRLDKLTVTSQN

SEQ ID NO: 227 - protein, 14 aa, Artificial Sequence
  Description of Artificial Sequence Synthetic peptide
TVTSQNLQLENLRM

SEQ ID NO: 228 - protein, 15 aa, Artificial Sequence
  Description of Artificial Sequence Synthetic peptide
LENLRMKLPKPPKPV

SEQ ID NO: 229 - protein, 15 aa, Artificial Sequence
  Description of Artificial Sequence Synthetic peptide
KLPKPPKPVSKMRMA

SEQ ID NO: 230 - protein, 9 aa, Artificial Sequence
  Description of Artificial Sequence Synthetic peptide
SKMRMATPL

SEQ ID NO: 231 - protein, 15 aa, Artificial Sequence
  Description of Artificial Sequence Synthetic peptide
LMQALPMGALPQGPM

SEQ ID NO: 232 - protein, 15 aa, Artificial Sequence
  Description of Artificial Sequence Synthetic peptide
LPQGPMQNATKYGNM

SEQ ID NO: 233 - protein, 15 aa, Artificial Sequence
  Description of Artificial Sequence Synthetic peptide
TKYGNMTEDHVMHLL

SEQ ID NO: 234 - protein, 15 aa, Artificial Sequence
  Description of Artificial Sequence Synthetic peptide
HVMHLLQNADPLKVY

SEQ ID NO: 235 - protein, 15 aa, Artificial Sequence
  Description of Artificial Sequence Synthetic peptide
DPLKVYPPLKGSFPE

SEQ ID NO: 236 - protein, 15 aa, Artificial Sequence
  Description of Artificial Sequence Synthetic peptide
KGSFPENLRHLKNTM

SEQ ID NO: 237 - protein, 15 aa, Artificial Sequence
  Description of Artificial Sequence Synthetic peptide
HLKNTMETIDWKVFE

SEQ ID NO: 238 - protein, 15 aa, Artificial Sequence
  Description of Artificial Sequence Synthetic peptide
DWKVFESWMHHWLLF

SEQ ID NO: 239 - protein, 15 aa, Artificial Sequence
  Description of Artificial Sequence Synthetic peptide
HHWLLFEMSRHSLEQ

SEQ ID NO: 240 - protein, 15 aa, Artificial Sequence
  Description of Artificial Sequence Synthetic peptide
RHSLEQKPTDAPPKE

SEQ ID NO: 241 - protein, 15 aa, Artificial Sequence
  Description of Artificial Sequence Synthetic peptide
DAPPKESLELEDPSS

SEQ ID NO: 242 - protein, 15 aa, Artificial Sequence
  Description of Artificial Sequence Synthetic peptide
LEDPSSGLGVTKQDL

SEQ ID NO: 243 - protein, 11 aa, Artificial Sequence
  Description of Artificial Sequence Synthetic peptide
VTKQDLGPVPM

SEQ ID NO: 244 - protein, 21 aa, Unknown
  Description of Unknown CXCR4 sequence
SEADDRYICDRFYPNDLWVVV

SEQ ID NO: 245 - protein, 15 aa, Unknown
  Description of Unknown CXCR4 sequence
DDRYICDRFYPNDLW

SEQ ID NO: 246 - protein, 15 aa, Artificial Sequence
  Description of Artificial Sequence Synthetic peptide
DLSNYSYSSTLPPFL

SEQ ID NO: 247 - protein, 13 aa, Artificial Sequence
  Description of Artificial Sequence Synthetic peptide
DLSNYSYSSTLPP

SEQ ID NO: 248 - protein, 11 aa, Artificial Sequence
  Description of Artificial Sequence Synthetic peptide
DLSNYSYSSTL

SEQ ID NO: 249 - protein, 9 aa, Artificial Sequence
  Description of Artificial Sequence Synthetic peptide
DLSNYSYSS

SEQ ID NO: 250 - protein, 7 aa, Artificial Sequence
  Description of Artificial Sequence Synthetic peptide
DLSNYSY

SEQ ID NO: 251 - protein, 5 aa, Artificial Sequence
  Description of Artificial Sequence Synthetic peptide
DLSNY

SEQ ID NO: 252 - protein, 11 aa, Artificial Sequence
  Description of Artificial Sequence Synthetic peptide
KVNGWIFGTFL

SEQ ID NO: 253 - protein, 9 aa, Artificial Sequence
  Description of Artificial Sequence Synthetic peptide
KVNGWIFGT

SEQ ID NO: 254 - protein, 7 aa, Artificial Sequence
  Description of Artificial Sequence Synthetic peptide
KVNGWIF

SEQ ID NO: 255 - protein, 5 aa, Artificial Sequence
  Description of Artificial Sequence Synthetic peptide
KVNGW

SEQ ID NO: 256 - protein, 13 aa, Artificial Sequence
  Description of Artificial Sequence Synthetic peptide
RRTVYSSNVSPAC

SEQ ID NO: 257 - protein, 11 aa, Artificial Sequence
  Description of Artificial Sequence Synthetic peptide
RRTVYSSNVSP

SEQ ID NO: 258 - protein, 9 aa, Artificial Sequence
  Description of Artificial Sequence Synthetic peptide
RRTVYSSNV

SEQ ID NO: 259 - protein, 7 aa, Artificial Sequence
  Description of Artificial Sequence Synthetic peptide
RRTVYSS

SEQ ID NO: 260 - protein, 5 aa, Artificial Sequence
  Description of Artificial Sequence Synthetic peptide
RRTVY

SEQ ID NO: 261 - protein, 13 aa, Artificial Sequence
  Description of Artificial Sequence Synthetic peptide
EDMGNNTANWRML

SEQ ID NO: 262 - protein, 11 aa, Artificial Sequence
  Description of Artificial Sequence Synthetic peptide
EDMGNNTANWR

SEQ ID NO: 263 - protein, 9 aa, Artificial Sequence
  Description of Artificial Sequence Synthetic peptide
EDMGNNTAN

SEQ ID NO: 264 - protein, 7 aa, Artificial Sequence
  Description of Artificial Sequence Synthetic peptide
EDMGNNT

SEQ ID NO: 265 - protein, 5 aa, Artificial Sequence
  Description of Artificial Sequence Synthetic peptide
EDMGN

SEQ ID NO: 266 - protein, 13 aa, Artificial Sequence
  Description of Artificial Sequence Synthetic peptide
MRTQVIQETCERR

SEQ ID NO: 267 - protein, 11 aa, Artificial Sequence
  Description of Artificial Sequence Synthetic peptide
MRTQVIQETCE

SEQ ID NO: 268 - protein, 9 aa, Artificial Sequence
  Description of Artificial Sequence Synthetic peptide
MRTQVIQET

SEQ ID NO: 269 - protein, 7 aa, Artificial Sequence
  Description of Artificial Sequence Synthetic peptide
MRTQVIQ

SEQ ID NO: 270 - protein, 5 aa, Artificial Sequence
  Description of Artificial Sequence Synthetic peptide
MRTQV

SEQ ID NO: 271 - protein, 13 aa, Artificial Sequence
  Description of Artificial Sequence Synthetic peptide
CERRNHIDRALDA

SEQ ID NO: 272 - protein, 11 aa, Artificial Sequence
  Description of Artificial Sequence Synthetic peptide
CERRNHIDRAL

SEQ ID NO: 273 - protein, 9 aa, Artificial Sequence
  Description of Artificial Sequence Synthetic peptide
CERRNHIDR

SEQ ID NO: 274 - protein, 7 aa, Artificial Sequence
  Description of Artificial Sequence Synthetic peptide
CERRNHI

SEQ ID NO: 275 - protein, 5 aa, Artificial Sequence
  Description of Artificial Sequence Synthetic peptide
CERRN

SEQ ID NO: 276 - protein, 13 aa, Artificial Sequence
  Description of Artificial Sequence Synthetic peptide
DRYICDRFYPNDL

SEQ ID NO: 277 - protein, 11 aa, Artificial Sequence
  Description of Artificial Sequence Synthetic peptide
DRYICDRFYPN

SEQ ID NO: 278 - protein, 9 aa, Artificial Sequence
  Description of Artificial Sequence Synthetic peptide
DRYICDRFY

SEQ ID NO: 279 - protein, 7 aa, Artificial Sequence
  Description of Artificial Sequence Synthetic peptide
DRYICDR

SEQ ID NO: 280 - protein, 5 aa, Artificial Sequence
  Description of Artificial Sequence Synthetic peptide
DRYIC

SEQ ID NO: 281 - protein, 13 aa, Artificial Sequence
  Description of Artificial Sequence Synthetic peptide
ICDRFYPNDLWVV

SEQ ID NO: 282 - protein, 7 aa, Artificial Sequence
  Description of Artificial Sequence Synthetic peptide
ICDRFYP

SEQ ID NO: 283 - protein, 5 aa, Artificial Sequence
  Description of Artificial Sequence Synthetic peptide
ICDRF

SEQ ID NO: 284 - protein, 13 aa, Artificial Sequence
  Description of Artificial Sequence Synthetic peptide
RFYPNDLWVVVFQ

SEQ ID NO: 285 - protein, 11 aa, Artificial Sequence
  Description of Artificial Sequence Synthetic peptide
RFYPNDLWVVV

SEQ ID NO: 286 - protein, 9 aa, Artificial Sequence
  Description of Artificial Sequence Synthetic peptide
RFYPNDLWV

SEQ ID NO: 287 - protein, 7 aa, Artificial Sequence
  Description of Artificial Sequence Synthetic peptide
RFYPNDL

SEQ ID NO: 288 - protein, 5 aa, Artificial Sequence
  Description of Artificial Sequence Synthetic peptide
RFYPN

SEQ ID NO: 289 - protein, 5 aa, Artificial Sequence
  Description of Artificial Sequence Synthetic peptide
NVPRA

SEQ ID NO: 290 - protein, 5 aa, Artificial Sequence
  Description of Artificial Sequence Synthetic peptide
SVPDG

SEQ ID NO: 291 - protein, 4 aa, Artificial Sequence
  Description of Artificial Sequence Synthetic peptide
LQDP